(19) United States
(12) Patent Application Publication
(10) Pub. No.: US 2026/0284034 A1
(43) Pub. Date: Sep. 24, 2026

(54) TREATMENT OF NON-SMALL CELL LUNG CANCER WITH A COMBINATION OF 3-AMINO-3-(1-METHYL-1H-IMIDAZOL-5-YL)-6-OXA-2(4,6)-QUINOLINA-1,4(1,3)-DIBENZENACYCLOHEXAPHANE-22,44-DICARBONITRILE AND AN EGFR-TKI

(71) Applicant: Kura Oncology, Inc., San Diego, CA (US)

(72) Inventors: Francis BURROWS, Solana Beach, CA (US); Shivani MALIK, San Diego, CA (US); Hetika VORA, San Diego, CA (US)

(21) Appl. No.: 19/476,479

(22) PCT Filed: Apr. 18, 2024
(86) PCT No.: PCT/US2024/025084

§ 371 Date: Oct. 17, 2025

Related U.S. Application Data
(60) Provisional application No. 63/497,390, filed on Apr. 20, 2023.
(60) Provisional application No. 63/504,390, filed on May 25, 2023.

(51) Int. Cl. A61K 31/4741; A61K 31/506; A61P 35/00

(52) U.S. Cl. CPC A61K 31/4741; A61K 31/506; A61P 35/00

(57) ABSTRACT

Provided herein are methods of using a compound of Formula (I), or a pharmaceutically acceptable form thereof, optionally in combination with an EGFR inhibitor, such as osimertinib, or a pharmaceutically acceptable salt thereof, for treating, preventing or managing non-small cell lung carcinoma (NSCLC).

### 1. CROSS-REFERENCE TO RELATED APPLICATIONS

This application claims the benefit of priority from U.S. Provisional Application Nos. 63/497,390, filed Apr. 20, 2023, and 63/504,390, filed May 25, 2023, each of which is incorporated by reference in its entirety.

### 2. FIELD

Provided herein are methods of using a farnesyltransferase inhibitor having a structure of Formula (I):

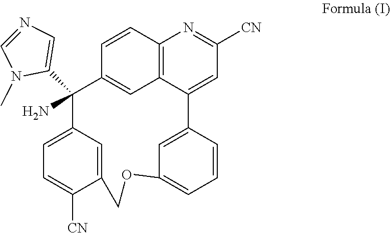 [SMILES: Cn1cncc1[C@@]1(N)c2ccc(C#N)c(c2)COc2cccc(c2)-c2cc(C#N)nc3ccc1cc23]

or a pharmaceutically acceptable form thereof, in combination with an epidermal growth factor receptor (EGFR) tyrosine kinase inhibitor (EGFR-TKI), such as osimertinib, or a pharmaceutically acceptable salt thereof, for treating non-small cell lung cancer (NSCLC). Also provided herein are methods of using the compound of Formula (I), or a pharmaceutically acceptable form thereof, for treating EGFR-mediated NSCLC.

### 3. BACKGROUND

Lung cancer is the second most prevalent cancer with approximately 2.2 million new cases per year representing 11.4% of all new cancer cases. It is the leading cause of death (1.8 million deaths) and 18.0% of cancer deaths (GLOBOCAN 2020, Number of new cases in 2020, both sexes, all ages, https://gco.iarc.fr/today/data/factsheets/cancers/15-Lung-fact-sheet.pdf). Non-small cell lung cancer (NSCLC), the most common type of lung cancer, occurs in approximately 83% of patients (Miller et al., Cancer J. Clin., 69 (5): 363-385 (2019)). Due to the asymptomatic nature of early-disease, many patients are diagnosed with later stages of disease (stage III-IV) and typically have short overall (OS) survival rates (Walters et al., Thorax, 68 (6): 551-564 (2013); Simeone et al., Future Oncology, 15.30:3491-3502 (2019)). In the United States (U.S.), approximately 55% of patients at initial diagnosis of NSCLC have distant metastases with a 7% 5-year survival rate, despite the recent development of novel anti-cancer therapies and regimens (Howlader et al., National Cancer Institute, (2020); American Cancer Society, https://www.cancer.org/cancer/lung-cancer/detection-diagnosis-staging/survival-rates.html (2021); Pacheco et al., J Thorac Oncol 14:691-700 (2019); Morgensztern et al., J Thorac Oncol 4:1524-1529 (2009); Uhlig et al., JAMA 2: e199702-e199702 (2019)).

Mutations in epidermal growth factor receptor (EGFR) Kirsten rat sarcoma viral oncogene (KRAS) and anaplastic lymphoma kinase, and (ALK) are frequently found in patients with NSCLC which has resulted in the development of targeted therapies (Liu et al., Am. J. Cancer Res., 7:187 (2017); Lee et al., J. Pathol. Transl. Med., 50:197 (2016)). However, EGFR is the main oncogenic driver in NSCLC, occurring in 10 to 50% of the patients depending on their origins, sex, and smoking habits.

EGFR-tyrosine kinase inhibitors (EGFR-TKI) are widely used to treat metastatic NSCLC in patients bearing EGFR-activating mutations, which are most commonly the L858R point mutation in exon 21 and a variety of deletions in exon 19 (Sharma et al., Nat. Rev. Cancer, 7 (3): 169-181 (2007). Approximately 60 to 70% of the EGFR-mutated patients respond to therapy, 20% have a stable disease and 15% develop primary resistance. Resistance mechanisms to EGFR-TKI include, but are not limited to, mediation by a bypass reactivation of one or several key proliferation and survival signaling pathways downstream from EGFR, mainly phosphoinositide 3-kinase (PI3K)/protein kinase B (AKT) (Engelman et al., Science, 316 (5827): 1039-43 (2007)), mitogen-activated protein kinase (MEK)/extracellular signal-regulated kinase (ERK) (Ercan et al., Cancer Discov., 2 (10): 934-47 (2012)), or STAT pathways (Lee et al., J. Cancer Sci. Ther., 6 (11): 468-477 (2014)). Initially it was thought that targeting the additional genetic alterations found in the tumor at the time of relapse could solve the problem of resistance (Thress et al., Nat. Med., 21 (6): 560 (2015)). However, inter- and intra-tumoral mutational heterogeneity has invalidated this strategy. There are at least three generations of EGFR-TKIs useful for treating or managing NSCLC; these include erlotinib and gefitinib (or first generation), as well as second and third generation EGFR-TKIs, such as afatinib, which are generally irreversible binders to EGFR. While these second and third generation inhibitors have been used to overcome resistance, such as resistance caused by T790M mutated EGFR, some patients treated with second or third generation EGFR-TKIs have also relapsed due to the emergence of new resistance mechanisms (e.g., EGFR-C797S/G mutation).

Osimertinib, a third-generation EGFR-TKI, has shown efficacy against activating EGFR mutations as well as the T790M-resistance mutations (see, e.g., Cross et al., Cancer Discov., 4 (9): 1046-1061 (2014) and Soria et al., N. Engl. J. Med., 378 (2): 113-125 (2017)). TAGRISSO® (also referred to as osimertinib or as osimertinib mesylate; see TAGRISSO®'s Jan. 19, 2022 label, available at https://www.accessdata.fda.gov/scripts/cder/daf/index.cfm. Also posted on AstraZeneca's website in “Prescribing Information” at https://www.tagrisso.com/) was approved by the United States Food and Drug Administration (“US FDA”) in 2015 for treating patients with metastatic EGFR-T790M mutant NSCLC whose disease had progressed after prior EGFR-TKI treatment. Subsequently, in 2018, the US FDA approved TAGRISSO® for treating TKI-naïve patients with metastatic NSCLC with tumors having an EGFR exon 19 deletion or an exon 21 L858R mutation. More recently, in 2020 the US FDA approved TAGRISSO® as an adjuvant therapy for use as a treatment after tumor resection of patients with NSCLC whose tumors have an EGFR exon 19 deletion or an exon 21 L858R mutation. Unfortunately, many patients will eventually develop secondary resistance to osimertinib leaving these patients with limited treatment options (see, e.g., Leonetti et al., Br. J. Cancer, 121:725-737 (2019); Schoenfeld et al., J. Thorac. Oncol., 15 (1): 18-21 (2020); and Eberlein et al., Cancer Res., 75 (12): 2489-500 (2015)). Mechanistic studies with EGFR-mutant NSCLC cell lines showed that treatment with osimertinib caused an inhibition of cell signaling mediators pERK and pRb. (Figarol et al., bioRxiv 2022, doi: 10.1101/2022.04.01.486707) However, as resistant proliferative clones emerged, pERK and pRb were reactivated. Additionally, RHOB and RHOE protein levels increased in a majority of the tested cell lines after long-term treatment with osimertinib.

Thirty percent of NSCLC patients carry EGFR mutations, and EGFR-TKIs are used frequently in these advanced NSCLC patients due to their superior activity and survival benefits compared to standard platinum chemotherapy. However, the emergence of drug resistance to EGFR-TKIs in patients with one or more EGFR-TKI-sensitizing mutations, such as an exon 19 deletion or an L858R mutation in exon 21, is well documented. While osimertinib (TAGRISSO®) was developed in part to overcome EGFR-TKI resistance in NSCLC patients, many of these patients will still develop a resistance to osimertinib (“osimertinib resistance”). In addition, NSCLC having an exon 20 insertion mutation (approximately 4-10% of EGFR-mutated population) is insensitive to EGFR-TKIs and such patients have a poor prognosis. Hou et al., Biomark. Res. 10, 21 (2022) (https://biomarkerres.biomedcentral.com/articles/10.1186/s40364-022-00372-6); see also Leal et al., Clin. Lung Cancer, 22 (6), e859-e869 (2021) (https://pubmed.ncbi.nlm.nih.gov/34127383/). Notably, while several EGFR-TKIs have been approved for NSCLC with a T790M mutation, an exon 19 deletion, and/or an L858R mutation, none have shown sufficient antitumor activity in the frontline setting to obtain approval for the exon 20 insertion mutation variant.

There remains a need in the art for therapies and regimens to treat NSCLC, including EGFR-mutated NSCLC, and advanced, metastatic, relapsed, or refractory forms of NSCLC. Similarly, there remains a need to reduce, avoid or overcome to the extent possible, drug resistance associated with existing therapies, including in the treatment of NSCLC. The combination therapies provided herein, comprising an EGFR-TKI and a compound of Formula (I), (II) or (III), or a pharmaceutically acceptable form thereof, mitigates one or more of the above-noted issues associated with treatments of NSCLC, such as mitigating drug resistance, or mitigating progression of the NSCLC, or combinations thereof.

### 4. SUMMARY

In one aspect, provided herein is a method of treating NSCLC in a patient, wherein the method comprises administering to the patient (a) a compound of Formula (I), (II), or (III), or a pharmaceutically acceptable form thereof (or a pharmaceutical composition comprising the same), preferably a compound of Formula (I) having a structure of:

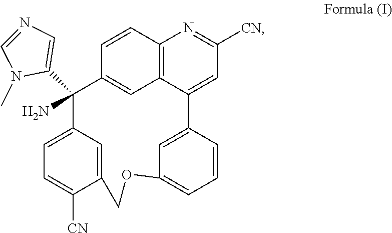 [SMILES: Cn1cncc1[C@@]1(N)c2ccc(C#N)c(c2)COc2cccc(c2)-c2cc(C#N)nc3ccc1cc23]

or a pharmaceutically acceptable form thereof, and (b) an EGFR-TKI, such as osimertinib, or a pharmaceutically acceptable salt thereof (or a pharmaceutical composition comprising the same).

In another aspect, provided herein is a method of mitigating EGFR-TKI resistance in an NSCLC patient currently or previously treated with an EGFR-TKI, comprising administering to the NSCLC patient a compound of Formula (I), (II), or (III), or a pharmaceutically acceptable form thereof, preferably a compound of Formula (I) or pharmaceutically acceptable form thereof (or a pharmaceutical composition comprising the same), as disclosed herein, alone or in combination with an EGFR-TKI such as osimertinib.

In another aspect, provided herein is a method of preventing or delaying emergence of EGFR-TKI resistance in an EGFR-TKI-naïve NSCLC patient, comprising administering to the patient (a) a compound of Formula (I), (II), or (III), or a pharmaceutically acceptable form thereof, preferably a compound of Formula (I), or a pharmaceutically acceptable form thereof (or a pharmaceutical composition comprising the same), as disclosed herein, before, during or after administering an EGFR-TKI such as osimertinib (or a pharmaceutical composition comprising the same).

In another aspect, provided herein is a method of treating EGFR-mediated NSCLC in a patient, comprising administering to the patient a compound of Formula (I), (II), or (III), or a pharmaceutically acceptable form thereof, preferably a compound of Formula (I), or a pharmaceutically acceptable form thereof (or a pharmaceutical composition comprising the same).

In another aspect, provided herein is a pharmaceutical composition comprising (a) a compound of Formula (I), or a pharmaceutically acceptable form thereof, and (b) an EGFR-TKI, preferably osimertinib, or a pharmaceutically acceptable salt thereof.

In another aspect, provided herein is a pharmaceutical kit, comprising (a) a compound of Formula (I), or a pharmaceutically acceptable form thereof, and (b) an EGFR-TKI, preferably osimertinib, or a pharmaceutically acceptable salt thereof.

In another aspect, provided herein is a pharmaceutical packaging, comprising (a) a compound of Formula (I), or a pharmaceutically acceptable form thereof, and (b) an EGFR-TKI, preferably osimertinib, or a pharmaceutically acceptable salt thereof.

In another aspect, provided herein is a pharmaceutical kit, comprising (a) a pharmaceutical composition comprising a compound of Formula (I), or a pharmaceutically acceptable form thereof, and a pharmaceutically acceptable carrier, excipient or diluent, and (b) a pharmaceutical composition comprising an EGFR-TKI, preferably osimertinib, or a pharmaceutically acceptable salt thereof, and a pharmaceutically acceptable carrier, excipient or diluent.

In another aspect, provided herein is a pharmaceutical packaging, comprising (a) a pharmaceutical composition comprising a compound of Formula (I), or a pharmaceutically acceptable form thereof, and a pharmaceutically acceptable carrier, excipient or diluent, and (b) a pharmaceutical composition comprising an EGFR-TKI, preferably osimertinib, or a pharmaceutically acceptable salt thereof, and a pharmaceutically acceptable carrier, excipient or diluent.

### 5. BRIEF DESCRIPTION OF THE FIGURES

FIGS. 1A-1C: Tumor volume over time in an EGFR-mutant NSCLC xenograft model under treatment with the compound of Formula (I) (Compound(S)-1) and osimertinib, alone and in combination. FIG. 1A: Control vehicle. FIG. 1B: Osimertinib. FIG. 1C: Compound(S)-1 at 1 mg/kg. FIG. 1D: Compound(S)-1 at 5 mg/kg. FIG. 1E: Compound(S)-1 at 20 mg/kg. The x/8 numbers on each figure indicate the number of animals out of 8 in each test group that were de novo resistant or that developed resistance.

FIG. 2: Tumor volume over time in an EGFR-mutant NSCLC xenograft model under treatment with the compound of Formula (I) and osimertinib, alone and in combination.

FIGS. 3A-3B: Cell growth over time in EGFR-mutant cells under treatment with osimertinib alone or in combination with the compound of Formula (I) (Compound(S)-1) or tipifarnib. FIG. 3A: All agents on a continuous dosing schedule. FIG. 3B: Osimertinib on a continuous dosing schedule and tipifarnib or Compound(S)-1 on an interval dosing schedule.

### 6. DETAILED DESCRIPTION

Unless defined otherwise, all technical and scientific terms used herein have the same meaning as is commonly understood by one of ordinary skill in the art. In the event that there are a plurality of definitions for a term herein, those in this section prevail unless stated otherwise.

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with doses, amounts, or weight percentages of ingredients of a composition or a dosage form, mean a dose, amount, or weight percent within 30%, within 20%, within 15%, within 10%, or within 5%, of the specified dose, amount, or weight percent.

It should be noted that if there is a discrepancy between a depicted structure and a name for that structure, the depicted structure is to be accorded more weight.

As used herein, a “pharmaceutically acceptable form” of compounds disclosed herein includes, but is not limited to, a pharmaceutically acceptable salt, solvate, isomer, and isotopologue (i.e., isotopically labeled derivative) of compounds disclosed herein. In some embodiments, a “pharmaceutically acceptable form” includes, but is not limited to, a pharmaceutically acceptable salt, solvate, isomer (e.g., tautomer or stereoisomer), and isotopologue (i.e., isotopically labeled derivative) of a compound of Formula (I), (II), or (III), as disclosed herein. As used herein, compounds disclosed herein include, but are not limited to, pharmaceutically acceptable salts, solvates, and/or isotopologues (i.e., isotopically labeled derivatives) of compounds disclosed herein. In some embodiments are contemplated a pharmaceutically acceptable salt, solvate, and/or isotopologue (i.e., isotopically labeled derivative) of the compound of Formula (I), (II), or (III), or osimertinib.

The term “isomer” as used herein comprises a stereoisomer or tautomer as defined herein. As used herein, the term “stereoisomers” is understood to mean isomers that differ only in the way the atoms are arranged in space. As used herein, the term “isomer” includes any and all geometric isomers and stereoisomers. For example, “isomers” include geometric double bond cis- and trans-isomers, also termed E- and Z-isomers; R- and S-enantiomers; diastereomers, (d)-isomers and (l)-isomers, racemic mixtures thereof; and other mixtures thereof, as falling within the scope of this disclosure.

As used herein and unless otherwise indicated, the term “stereoisomer” or “stereoisomerically pure” means one stereoisomer of a compound that is substantially free of other stereoisomers of that compound. For example, a stereoisomerically pure compound having one chiral center will be substantially free of the opposite enantiomer of the compound. For example, stereoisomerically pure compound of Formula (I) (i.e., (S)-3-amino-3-(1-methyl-1H-imidazol-5-yl)-6-oxa-2 (4,6)-quinolina-1,4 (1,3)-dibenzenacyclohexaphane-2²,4⁴-dicarbonitrile, or Compound(S)-1), substantially free of a compound of Formula (II) (i.e., (R)-3-amino-3-(1-methyl-1H-imidazol-5-yl)-6-oxa-2 (4,6)-quinolina-1,4 (1,3)-dibenzenacyclohexaphane-2²,4⁴-dicarbonitrile, or Compound (R)-2). A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments provided herein, including mixtures thereof.

It is to be understood that the compounds provided herein may contain chiral centers. Such chiral centers may be of either the (R) or(S) configuration, or may be a mixture thereof. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its(S) form.

Optically active (+) and (−), (R)- and(S)-, or (D)- and (L)-isomers may be prepared using chiral synthons or chiral reagents, or resolved using conventional techniques, such as chromatography on a chiral stationary phase.

The use of stereoisomerically pure forms of such compounds, as well as the use of mixtures of those forms, are encompassed by the embodiments provided herein. For example, mixtures comprising equal or unequal amounts of the enantiomers of a particular compound may be used in methods and compositions provided herein. These isomers may be asymmetrically synthesized or resolved using standard techniques such as chiral columns or chiral resolving agents. See, e.g., Jacques, J., et al., (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuja, S., Chiral Separation Methods for Pharmaceutical and Biotechnological Products (John Wiley & Sons, 2011).

In certain embodiments, the pharmaceutically acceptable form is a tautomer. As used herein, the term “tautomer” is a type of isomer that includes two or more interconvertable compounds resulting from at least one formal migration of a hydrogen atom and at least one change in valency (e.g., a single bond to a double bond, a triple bond to a double bond, or a triple bond to a single bond, or vice versa). “Tautomerization” includes prototropic or proton-shift tautomerization, which is considered a subset of acid base chemistry. “Prototropic tautomerization” or “proton-shift tautomerization” involves the migration of a proton accompanied by changes in bond order. The exact ratio of the tautomers depends on several factors, including temperature, solvent, and pH. Where tautomerization is possible (e.g., in solution), a chemical equilibrium of tautomers can be reached. Tautomerizations (i.e., the reaction providing a tautomeric pair) can be catalyzed by acid or base, or can occur without the action or presence of an external agent. The concentrations of the isomeric forms will depend on the environment the compound is found in and may be different depending upon, for example, whether the compound is a solid or is in an organic or aqueous solution. Exemplary tautomerizations include, but are not limited to, keto-enol; amide-imide; lactam-lactim; enamine-imine; and enamine-(a different) enamine tautomerizations. For example, in aqueous solution, pyrazoles may exhibit the following isomeric forms, which are referred to as tautomers of each other:

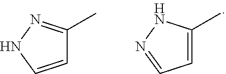

As readily understood by one skilled in the art, a wide variety of functional groups and other structures may exhibit tautomerism and all tautomers of a compound are within the scope of the compound as provided herein.

The term “isotopologue” refers to isotopically-enriched compounds that are identical to those recited herein, but for the fact that one or more atoms are replaced by an atom having an atomic mass or mass number different from the atomic mass or mass number usually found in nature. Unless otherwise stated, structures depicted herein are also meant to include compounds which differ only in the presence of one or more isotopically enriched atoms. Examples of isotopes that can be incorporated into compounds described herein include isotopes of hydrogen, carbon, nitrogen, oxygen, phosphorus, fluorine and chlorine, such as ²H, ³H, ¹³C, ¹⁴C, ¹⁵N, ¹⁷O, ¹⁸O, ³²P, ³³P, ³³S, ³⁴S, ³⁵S, ³⁶S, ¹⁸F, ³⁵Cl, ³⁶Cl, and ³⁷Cl, respectively, each of which is also within the scope of this description. For example, compounds having the present structures except for the replacement or enrichment of a hydrogen by deuterium or tritium at one or more atoms in the molecule, are within the scope of this disclosure. In one embodiment, provided herein are isotopically labeled compounds having one or more hydrogen atoms replaced by or enriched by deuterium. In one embodiment, provided herein are isotopically labeled compounds having one or more hydrogen atoms replaced by or enriched by tritium. Further, substitution with heavier isotopes such as deuterium (i.e., 2H) can afford certain therapeutic advantages resulting from greater metabolic stability (e.g., increased in vivo half-life or reduced dosage requirements). Isotopically labeled compounds disclosed herein can generally be prepared by substituting an isotopically labeled reagent for a non-isotopically labeled reagent. Isotopically-enriched compounds of Formula (I), (II), or (III), or a pharmaceutically acceptable form thereof, can generally be prepared using procedures known to persons of ordinary skill in the art by substituting an appropriate isotopically-enriched reagent for a non-isotopically-enriched reagent.

When the compounds are enriched with deuterium, the deuterium-to-hydrogen ratio on the deuterated atoms of the molecule substantially exceeds the naturally occurring deuterium-to-hydrogen ratio.

An embodiment described herein may include an isotopologue form of the compound of Formula (I), (II), or (III), or a pharmaceutically acceptable form thereof, wherein the isotopologue is substituted on one or more atom members of said compound, or a pharmaceutically acceptable form thereof, with one or more deuterium atoms in place of one or more hydrogen atoms. An embodiment described herein may include a compound of Formula (I), (II), or (III), or a pharmaceutically acceptable form thereof, wherein a carbon atom may have from 1 to 3 hydrogen atoms optionally replaced with deuterium.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases, such as suitable inorganic and organic addition acids and bases.

In certain embodiments, the pharmaceutically acceptable form of the compounds disclosed herein is exclusive of a salt form (i.e., is not a salt), sometimes referred to as a freebase form, of the compounds disclosed herein. For example, in one embodiment, the pharmaceutically acceptable form of a compound of Formula (I), (II), or (III), as disclosed herein, is exclusive of a salt form and includes a pharmaceutically acceptable solvate, isomer, and isotopologue (i.e., isotopically labeled derivative) of the compound of Formula (I), (II), or (III), as disclosed herein, such as exclusive of a salt form and a hydrate, stereoisomer, and isotopologue of the compound of Formula (I), (II), or (III), as disclosed herein.

In certain embodiments, the pharmaceutically acceptable form is a solvate (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. In some embodiments, the solvate is a hydrate. Pharmaceutically acceptable solvates and hydrates are complexes that, for example, can include 0.1, 0.25, 0.50, 0.75, or 1 solvent or water molecules, or can include 1 to about 100, or 1 to about 10, or one to about 2, about 3 or about 4, solvent or water molecules. It will be understood that the term “compound” as used herein encompasses the compound and solvates of the compound, as well as mixtures thereof.

As used herein and unless otherwise indicated, the term “therapeutically effective amount” or “effective amount” in connection with a compound means an amount capable of achieving the target effect, such as treating a disorder, disease or condition, or symptoms thereof, or mitigating, preventing emergence of, or delaying emergence of drug resistance. In some embodiments, an effective amount of the compound of Formula (I), an effective amount of an EGFR-TKI such as osimertinib, and/or an effective amount in the context of a combination thereof, can provide one or more benefits according to the methods provided herein. For example, the effective amount of the compound of Formula (I), the effective amount of the EGFR-TKI such as osimertinib, and/or the effective amount in the context of a combination thereof, can treat one or more symptoms associated with NSCLC; can prevent or delay emergence of osimertinib resistance in an NSCLC; can mitigate EGFR-TKI or osimertinib resistance in an NSCLC; can provide inhibition of disease progression, inhibition of tumor growth, reduction of primary tumor, relief of tumor-related symptoms, inhibition of tumor secreted factors, delayed appearance of primary or secondary tumors, delaying time to emergence of drug resistance, slowed development of primary or secondary tumors, decreased occurrence of primary or secondary tumors, slowed or decreased severity of secondary effects of disease, arrested tumor growth and regression of tumors, increased Time to Progression (TTP), increased Progression-Free Survival (PFS), increased Overall Survival (OS), increased overall response rate (ORR), increased duration of response (DoR), or decreased time to response (TTR), or combinations thereof.

As used herein, the term “non-small cell lung cancer” or “NSCLC” has its general meaning in the art and refers to a disease in tissues of the lung involving uncontrolled cell growth, which, in some cases, leads to metastasis. The majority of primary lung cancers are carcinomas of the lung, derived from epithelial cells. The main types of lung cancer are small cell lung carcinoma (SCLC) and non-small cell lung carcinoma (NSCLC). In a particular embodiment, the subject suffers from a non-small cell lung cancer. As used herein, the term “non-small cell lung cancer,” also known as non-small cell lung carcinoma (NSCLC), refers to epithelial lung cancer other than small cell lung carcinoma (SCLC). NSCLC may include adenocarcinoma or squamous cell carcinoma. Other less common types of non-small cell lung cancer include pleomorphic, carcinoid tumor, salivary gland-type carcinoma, and unclassified carcinoma, or large cell carcinoma, neuroendocrine carcinoma, or sarcomatoid carcinoma. In some aspects, NSCLC may have squamous histology. NSCLC may be categorized using the tumor-node metastasis (TNM) staging system. See Spira, J. & Ettinger, D. S., N. Engl. J. Med., 350:382-(2004); Greene et al (eds). AJCC Cancer Staging Manual. 6^{th} edition. New York: Springer-Verlag, 2002:167-77; Sobin, L. H., & C. H. Wittekind (eds). International Union Against Cancer. TNM classification of malignant tumors. 6th edition. New York: Wiley-Liss (2002). Accordingly, in some embodiments, the NSCLC may be stratified into any of the preceding stages (e.g., occult, stage 0, stage IA, stage IB, stage IIA, stage IIB, stage IIIA, stage IIIB or stage IV).

In some embodiments, the NSCLC is a solid tumor. In some embodiments, the NSCLC is metastatic NSCLC, advanced NSCLC, relapsed NSCLC, or refractory NSCLC. In some embodiments, the NSCLC is metastatic NSCLC or advanced NSCLC. In some embodiments, the NSCLC is relapsed NSCLC or refractory NSCLC. In some embodiments, the NSCLC is an EGFR-mediated NSCLC. In some embodiments, the NSCLC is an EGFR-mutant mediated NSCLC. In some embodiments, the EGFR-mutant mediated NSCLC has an exon 19 deletion, an exon 21 L858R mutation, a T790M mutation, an exon 20 insertion, or a C797S mutation in EGFR. For example, in some embodiments, the NSCLC has two or more, or three or more, EGFR mutations selected from the group consisting of an exon 19 deletion, an exon 21 L858R mutation, a T790M mutation, an exon 20 insertion, and a C797S mutation in EGFR. In some embodiments, the EGFR-mutant mediated NSCLC is or comprises an exon 19 deletion in EGFR. In some embodiments, the EGFR-mutant mediated NSCLC is or comprises an exon 21 L858R mutation in EGFR. In some embodiments, the EGFR-mutant mediated NSCLC is or comprises a T790M mutation in EGFR. In some embodiments, the EGFR-mutant mediated NSCLC is or comprises an exon 20 insertion in EGFR. In some embodiments, the EGFR-mutant mediated NSCLC is or comprises a C797S mutation in EGFR. In some embodiments, the NSCLC is an EGFR-TKI resistant NSCLC, such as an osimertinib-resistant NSCLC. In some embodiments, the EGFR-mediated NSCLC is not Ras-dependent, or not K-Ras dependent, or not H-Ras dependent, or not N-Ras dependent, or a combination thereof, for example is K-Ras wild type and/or H-Ras wild type (e.g., not mutant, overexpressed, or amplified).

In some embodiments, the exon 20 insertion mutation is a post C-helix insertion of 1 to 4 amino acids. These mutations account for 80-90% of all ex20ins mutations. In some embodiments, the exon 20 insertion mutation is A763_Y764insFQEA, A763_Y764insFQEA, Y764_V765insHH, M766_A767insAI, V769_D770insASV, D770_N771insNPG, D770 N77linsNPG, D770_N77lins_SSVD, or H773 (see Hou et al., Biomark. Res. 10, 21 (2022)).

As used herein, the term “pharmaceutically acceptable carrier, excipient or diluent” means a carrier, excipient or diluent approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which a therapeutic agent is administered. Such pharmaceutical carriers can be sterile liquids, such as water and oils, including those of petroleum, animal, vegetable or synthetic origin, such as peanut oil, soybean oil, mineral oil, sesame oil and the like. Water is a specific carrier for intravenously administered pharmaceutical compositions. Saline solutions and aqueous dextrose and glycerol solutions can also be employed as liquid carriers, particularly for injectable solutions. For example, the term pharmaceutically acceptable carrier, excipient or diluent includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions as disclosed herein is contemplated. Supplementary active ingredients can also be incorporated into the pharmaceutical compositions. Examples of excipients that can be used in oral dosage forms provided herein include, but are not limited to, binders, fillers, disintegrants, and lubricants.

As used herein, the terms “prevention” and “preventing” refer to obtaining beneficial or desired results including, but not limited, to prophylactic benefit. For prophylactic benefit, the compounds and pharmaceutical compositions disclosed herein can be administered according to the methods provided herein to a patient at risk of developing NSCLC, to a patient reporting one or more of the physiological symptoms of NSCLC, even though a diagnosis of the NSCLC may not have been made, or to a patient in remission from NSCLC.

As used herein, the terms “continuous dosing” and “continuous dosing schedule,” or “continuous” and “continuously” in the context of administering, refer to daily administration, such as once daily (QD), twice daily (BID), three times daily (TID), or four times a day (QID), of the compound of Formula (I), or the EGFR-TKI, preferably osimertinib, or a combination thereof, as disclosed herein.

As used herein, the terms “concurrently” or “concurrent,” in the context of an administration, refer to a co-administration of two or more agents, such as the compound of Formula (I) and an EGFR-TKI such as osimertinib, to a subject during a single day and conducted in close proximity in time of one another during that day. For example, in certain embodiments, the concurrent administration of two or more agents to a subject is conducted within 3 hours, 2 hours, 1 hour, 30 minutes, or simultaneously, during a single day.

As used herein, the terms “sequentially” or “sequential,” in the context of an administration, refer to a co-administration of two or more agents, such as such as the compound of Formula (I) and an EGFR-TKI such as osimertinib, in a particular order, such as in a scheduled order, to a subject during a single day. For example, in certain embodiments, the concurrent administration of two agents to a subject, such as such as the compound of Formula (I) and an EGFR-TKI such as osimertinib, to a subject, is conducted such that one agent is first administered to the subject followed by administration of the second agent to the subject on the same day, with no specific time limit unless otherwise specified, during the same day.

As used herein, the terms “interval dosing” and “interval dosing schedule” refer to a schedule of administering an agent on certain days and not administering the agent on other days during the course of a treatment cycle, such as during a 28-day treatment cycle. For example, interval dosing of an agent, such as such as the compound of Formula (I) and optionally an EGFR-TKI such as osimertinib, includes scheduled periods of dosing of an agent followed by scheduled periods of a drug holiday of the agent during the course of a treatment cycle, such as during a 28-day treatment cycle. For example, interval dosing of an agent includes, but is not limited to, administering the agent only every other day, administering the agent continuously only every other week (e.g., one week on and one week off, or vice versa, such as continuously on days 1-7 and 15-21 of a 28-day treatment cycle, or days 8-14 and 22-28 of a 28-day treatment cycle), administering the agent continuously only for two consecutive weeks (e.g., two weeks on and two weeks off, or vice versa, such as continuously on days 1-14 of a 28-day treatment cycle, days 7-21 of a 28-day treatment cycle, or days 15-28 of a 28-day treatment cycle), or administering the agent continuously only for three consecutive weeks (e.g., three weeks on and one week off, or vice versa, such as continuously on days 1-21 of a 28-day treatment cycle, or days 7-28 of a 28-day treatment cycle), during a treatment cycle, such as during a 28-day treatment cycle. For example, in certain embodiments, such as the compound of Formula (I) and osimertinib, as disclosed herein, may be administered only every other day, continuously only every other week, or continuously only for week 1, weeks 2, or weeks 3, during the course of a treatment cycle, such as during a 28-day treatment cycle. In certain embodiments, the agent is the compound of Formula (I) as disclosed herein. In certain embodiments, the agent is osimertinib as disclosed herein.

A “treatment cycle” as understood herein is a given period of time during which one or more treatments are administered to a subject in need thereof. In some embodiments, a treatment cycle is a 28-day treatment cycle.

As used herein, the terms “delayed dosing,” “delayed dosing period,” and “delayed dosing schedule” refer to the period of time between administering an initial dose of osimertinib to an osimertinib or EGFR-TKI naïve subject and subsequently administering an initial dose of the compound of Formula (I) to the subject, or between administering an initial dose of the compound of Formula (I) to a relapsed or refractory NSCLC subject previously, but not currently being, treated with osimertinib or an EGFR-TKI and subsequently administering an initial dose of the compound of Formula (I) to the relapsed or refractory NSCLC subject. For example, a delayed dosing of an initial dose of the compound of Formula (I) to an EGFR-TKI or osimertinib naïve subject or a relapsed or refractory NSCLC subject previously, but not currently being, treated with an EGFR-TKI or osimertinib includes a delay of about 1 week, about 2 weeks, about 3 weeks, about 4 weeks, about 1 month, about 5 weeks, about 6 weeks, about 7 weeks, about 8 weeks, about 2 months, about 3 months, about 4 months, about 5 months, about 6 months, about 7 months, about 8 months, about 9 months, about 10 months, about 11 months, about 12 months, about 1.25 years, about 1.5 years, about 1.75 years, about 2 years, about 2.25 years, about 2.5 years, about 2.75 years, or about 3 years, following the initial dose of an EGFR-TKI such as osimertinib to the EGFR-TKI or osimertinib naïve subject or the or relapsed or refractory NSCLC subject previously, but not currently being, treated with an EGFR-TKI such as osimertinib. For example, in certain embodiments, a delayed dosing of an initial dose of the compound of Formula (I) is no longer than about 12 months, about 9 months, about 6 months, about 3 months, about 2 months, about 8 weeks, about 6 weeks, about 5 weeks, about 1 month, about 4 weeks, about 3 weeks, about 2 weeks, or about 1 week, following the initial dose of an EGFR-TKI such as osimertinib to the EGFR-TKI or osimertinib naïve subject or the or relapsed or refractory NSCLC subject previously, but not currently being, treated with an EGFR-TKI such as osimertinib. In some embodiments, the delayed dosing schedule comprises administering the compound of Formula (I) on an interval dosing schedule after the delay period of time.

As used herein, the terms “dose escalation,” “dose escalation interval,” “escalation dosing,” “escalation dosing period,” and “escalation dosing schedule,” refer to a step-wise increase in the amount of an agent, such as the compound of Formula (I) or an EGFR-TKI such as osimertinib, administered to a subject over a period of time (sometimes referred to herein as a dose escalation period of time). In certain embodiments, the step-wise increase is an increase in the dose amount of the agent administered to the subject. In certain embodiments, the step-wise increase is an increase in the dose per day of the agent administered to the subject. In certain embodiments, the period of time over which completion of the step-wise increase occurs (the dose escalation period of time) is 2 days, 3 days, 7 days (1 week), 10 days, 2 weeks, 3 weeks, or 4 weeks. In certain embodiments, a step-wise increase in the amount of the agent occurs (or is scheduled to occur) every 1 day, 2 days, 3 days, 7 days (1 week), 10 days, or 2 weeks, during the dose escalation period of time. In certain embodiments, the increase, such as the step-wise increase or the total increase, in the amount of the agent administered to the subject is a 10%-99%, such as 10%, 25%, 30%, 33%, 50%, 66%, 75%, 90%, 1.5 fold, 2 fold, 2.5 fold, 3 fold, 3.5 fold, or 4 fold increase in the amount of the agent administered to the subject, relative to an initial amount of the agent administered to the subject at the beginning of the dose escalation period of time, or relative to the prior step-wise increased amount of the agent administered to the subject during the dose escalation period of time. In certain embodiments, the final amount of an agent administered at the end of the dose escalation period of time is an effective amount of the agent, such as the effective amount of the agent administered during a treatment cycle, such as a 28-day treatment cycle. In certain embodiments, the amount of only one agent of a combination of agents administered to a subject is increased step-wise over the course of the dose escalation period of time while the amounts of remaining agents of the combination are held constant. For example, in certain embodiments, the amount of the compound of Formula (I) administered to a subject is increased over the course of the dose escalation period of time while the amount of osimertinib administered to the subject is held constant. In certain embodiments, the amount of a first agent, such as the compound of Formula (I), and the amount of a second agent, such as an EGFR-TKI such as osimertinib, of a combination of agents administered to a subject are each independently increased step-wise over the course of the dose escalation period of time. In certain embodiments, the agent is the compound of Formula (I) as disclosed herein. In certain embodiments, the agent is osimertinib as disclosed herein.

As used herein, the terms “dose reduction,” “dose reduction interval,” “reduced dosing,” “dose reduction period,” and “dose reduction schedule,” refer to a step-wise decrease in the amount of an agent, such as the compound of Formula (I) or an EGFR-TKI such as osimertinib, administered to a subject over a period of time (sometimes referred to herein as a dose reduction period of time). In certain embodiments, the step-wise decrease is a decrease in the dose amount of the agent administered to the subject. In certain embodiments, the step-wise decrease is a decrease in the dose per day of the agent administered to the subject. In certain embodiments, the period of time over which completion of the step-wise decrease occurs (the dose reduction period of time) is 2 days, 3 days, 7 days (1 week), 10 days, 2 weeks, 3 weeks, or 4 weeks. In certain embodiments, a step-wise decrease in the amount of the agent occurs (or is scheduled to occur) every 1 day, 2 days, 3 days, 7 days (1 week), 10 days, or 2 weeks, during the dose reduction period of time. In certain embodiments, the decrease, such as the step-wise decrease or the total decrease, in the amount of the agent administered to the subject is a 10%-99%, such as 10%, 25%, 30%, 33%, 50%, 66%, 75%, 90%, 1.5 fold, 2 fold, 2.5 fold, 3 fold, 3.5 fold, or 4 fold decrease in the amount of the agent administered to the subject, relative to an initial amount of the agent administered to the subject at the beginning of the dose reduction period of time, or relative to the prior step-wise decreased amount of the agent administered to the subject during the dose reduction period of time. In certain embodiments, the final amount of an agent administered at the end of the dose reduction period of time is an effective amount of the agent, such as the effective amount of the agent administered during a treatment cycle, such as a 28-day treatment cycle. In certain embodiments, the amount of only one agent of a combination of agents administered to a subject is decreased step-wise over the course of the dose reduction period of time while the amounts of remaining agents of the combination are held constant. For example, in certain embodiments, the amount of the compound of Formula (I) administered to a subject is decreased over the course of the dose reduction period of time while the amount of an EGFR-TKI such as osimertinib administered to the subject is held constant. In certain embodiments, the amount of a first agent, such as the compound of Formula (I), and the amount of a second agent, such as an EGFR-TKI such as osimertinib, of a combination of agents administered to a subject are each independently decreased step-wise over the course of the dose reduction period of time. In certain embodiments, the agent is the compound of Formula (I) as disclosed herein. In certain embodiments, the agent or EGFR-TKI is osimertinib as disclosed herein.

As used herein, the terms or “loading dosing cycle” refer to administering a higher dose (sometimes referred to herein as a “loading dose”) of an agent, such as the compound of Formula (I) or an EGFR-TKI such as osimertinib, than the maintenance dose (e.g., the dose administered during a treatment cycle). In certain embodiments, the loading dosing cycle continues until a therapeutic steady-state concentration of the agent is achieved. In certain embodiments, a loading dose of an agent, such as the compound of Formula (I) or an EGFR-TKI such as osimertinib, can range from about 1.1 to about 10 times the dose of the agent administered during a treatment cycle. In certain embodiments, a loading dose per day of an agent, such as the compound of Formula (I) or an EGFR-TKI such as osimertinib, can range from about 1.1 to about 10 times the dose per day of the agent administered during a treatment cycle. In certain embodiments, the agent is the compound of Formula (I) as disclosed herein. In certain embodiments, the agent or EGFR-TKI is osimertinib as disclosed herein.

As used herein and unless otherwise indicated, the term “subject” to which administration is contemplated, can be an animal, including, but not limited to, a human (e.g., a male or female of any age group, such as an adult subject or an adolescent subject); primates (e.g., cynomolgus monkeys, rhesus monkeys), and/or other mammals, including commercially relevant mammals such as cattle, pigs, horses, sheep, goats, cats, dogs, rabbits, rodents, and/or birds (e.g., commercially relevant birds such as chickens, ducks, geese, quail, and/or turkeys). In some embodiments, the subject is a mammal. In some embodiments, the subject is a human. In some embodiments, the subject is an adolescent human. In some embodiments, the subject is an adult human. In some embodiments, the subject is a patient, for example, a human patient. In some embodiments, the subject is a smoker. In some embodiments, the subject is a non-smoker. In some embodiments, the subject is a non-smoker who had previously been a smoker.

In some embodiments, the subject has, suffers from, has symptoms associated with, or is diagnosed as having, NSCLC. In some embodiments, the subject has or suffers from NSCLC. In some embodiments, the subject has symptoms associated with NSCLC. In some embodiments, the subject is diagnosed as having NSCLC. In some embodiments, the subject may be diagnosed as having NSCLC by one skilled in the art, for example, a physician, such as an oncologist. In some embodiments, the subject may be diagnosed as having NSCLC by analysis of plasma or a tissue biopsy from the subject, such as a tumor tissue biopsy. In some embodiments, the subject may be diagnosed as having NSCLC by one or more imaging tests (e.g., MRI, CT, PET, PET-CT, nuclear scan, ultrasound), optionally in combination with analysis of plasma or tumor tissue biopsy. In some embodiments, the subject may be diagnosed as having NSCLC by a blood analysis. In some embodiments the analysis includes a ctDNA analysis. In some embodiments, the subject is a previously treated NSCLC subject. In some embodiments, the subject is an EGFR TKI naïve subject. In some embodiments, the subject is an osimertinib naïve subject. In some embodiments, the subject has been treated previously with an EGFR TKI. In some embodiments, the subject has been treated previously with osimertinib. In some embodiments, the subject is currently being treated with an EGFR TKI, such as currently being treated with osimertinib (mesylate). In some embodiments, the NSCLC subject is an EGFR-TKI resistant NSCLC subject, such as an osimertinib-resistant NSCLC subject. In some embodiments, the subject is a NSCLC subject in remission. In some embodiments, the NSCLC subject has early stage NSCLC, metastatic NSCLC, advanced NSCLC, relapsed NSCLC, or refractory NSCLC. In some embodiments, the NSCLC subject has early stage NSCLC. In some embodiments, the NSCLC subject has metastatic NSCLC or advanced NSCLC. In some embodiments, the NSCLC subject has relapsed NSCLC or refractory NSCLC. In some embodiments, the NSCLC has squamous histology.

In some embodiments, the NSCLC subject has an EGFR-mediated NSCLC. In some embodiments, the NSCLC subject has an EGFR-mutant mediated NSCLC. In some embodiments, the subject may be diagnosed as having an EGFR-mutant mediated NSCLC by detection and analysis with an FDA-approved test, such as a Cobas® EGFR Mutation Test, or by tumor tissue or plasma (ctDNA) assessment). In some embodiments, the NSCLC subject has an EGFR-mutant mediated NSCLC selected from the group consisting of an exon 19 deletion, an exon 21 L858R mutation, a T790M mutation, an exon 20 insertion, and a C797S mutation in EGFR. For example, in some embodiments, the NSCLC subject has an EGFR-mutant mediated NSCLC that has two or more, or three or more, EGFR mutations selected from the group consisting of an exon 19 deletion, an exon 21 L858R mutation, a T790M mutation, an exon 20 insertion, and a C797S mutation in EGFR. In some embodiments, the NSCLC subject has EGFR-TKI sensitive mutations, such as an exon 19 deletion, an exon 21 L858R mutation, or combinations thereof. In some embodiments, the EGFR-mutant mediated NSCLC is or comprises an exon 19 deletion in EGFR. In some embodiments, the EGFR-mutant mediated NSCLC is or comprises an exon 21 L858R mutation in EGFR. In some embodiments, the EGFR-mutant mediated NSCLC is or comprises a T790M mutation in EGFR. In some embodiments, the EGFR-mutant mediated NSCLC is or comprises an exon 20 insertion in EGFR. In some embodiments, the EGFR-mutant mediated NSCLC is or comprises a C797S mutation in EGFR. In some embodiments, the EGFR-mutant mediated NSCLC has or comprises an exon 20 insertion mutation in EGFR, and comprises one or more further EGFR mutations.

As used herein and unless otherwise indicated, the terms “treat,” “treating,” and “treatment,” are used interchangeably herein, and means an alleviation or amelioration, in whole or in part, of a disorder, disease or condition, such as NSCLC, or one or more of the symptoms associated with a disorder, disease, or condition, such as NSCLC, or slowing or halting of further progression or worsening of those symptoms, or alleviating or eradicating the cause(s) of the disorder, disease, or condition itself, such as NSCLC. In some embodiments, these terms refer to an approach for obtaining beneficial or desired results including, but not limited to, a therapeutic benefit or a prophylactic benefit. A therapeutic benefit resulting from the methods provided herein includes the eradication or amelioration of the underlying disorder, such as NSCLC, being treated, the eradication or amelioration of one or more of the physiological symptoms associated with the underlying disorder (e.g., NSCLC) such that an improvement is observed in the patient, notwithstanding that the patient can still be afflicted with the underlying disease or disorder (e.g., NSCLC). For example, when used in reference to a patient having NSCLC, refers to an action that reduces the severity of the NSCLC, or retards or slows the progression of the NSCLC, including (a) inhibiting the NSCLC growth, or arresting development of the NSCLC, and (b) causing regression of the NSCLC, or delaying or minimizing one or more symptoms associated with the presence of the NSCLC. A prophylactic benefit resulting from the methods provided herein includes delaying or eliminating the appearance of a disease or disorder (e.g., NSCLC), delaying or eliminating the onset of symptoms of a disease or disorder (e.g., NSCLC), slowing, halting, or reversing the progression of a disease or disorder (e.g., NSCLC), or any combination thereof.

In the context of a non-small cell lung cancer, treatment may be assessed by inhibition of disease progression, inhibition of tumor growth, reduction of primary tumor, relief of tumor-related symptoms, inhibition of tumor secreted factors, delayed appearance of primary or secondary tumors, delaying time to emergence of drug resistance, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS), among others, such as objective response rate (ORR) and disease control rate (DCR). OS as used herein means the time from treatment onset until death from any cause. TTP, as used herein, means the time from treatment onset until tumor progression; TTP does not include deaths. In some embodiments, PFS means the time from treatment onset until tumor progression or death. In some embodiments, PFS means the time from the first dose of compound to the first occurrence of disease progression or death from any cause. In some embodiments, PFS rates are computed using the Kaplan-Meier estimates. Event-free survival (EFS) means the time from treatment onset until any treatment failure, including disease progression, treatment discontinuation for any reason, or death. In some embodiments, overall response rate (ORR) means the percentage of patients who achieve a response. In some embodiments, ORR means the sum of the percentage of patients who achieve complete and partial responses. In some embodiments, ORR means the percentage of patients whose best response ≥partial response (PR). In some embodiments, DCR is the percentage of patients whose disease shrinks or remains stable over a certain time period. DCR is the sum of the complete, partial and stable disease rates. In some embodiments, duration of response (DoR) is the time from achieving a response until relapse or disease progression. In some embodiments, DoR is the time from achieving a response ≥partial response (PR) until relapse or disease progression. In some embodiments, DoR is the time from the first documentation of a response until the first documentation of progressive disease or death. In some embodiments, DoR is the time from the first documentation of a response ≥partial response (PR) until to the first documentation of progressive disease or death. In some embodiments, time to response (TTR) means the time from the first dose of compound to the first documentation of a response. In some embodiments, TTR means the time from the first dose of compound to the first documentation of a response ≥partial response (PR).

As used herein, the terms “mitigate” and “mitigating” with respect to resistance to a therapy includes slowing or delaying the time to drug resistance, preventing drug resistance from occurring, or reducing or overcoming drug resistance to a different agent.

### 6.1 Compounds

In some embodiments, the methods provided herein include administering (a) a farnesyltransferase inhibitor and (b) an EGFR tyrosine kinase inhibitor (TKI) to a subject. In some embodiments, the farnesyltransferase inhibitor is a selective farnesyltransferase inhibitor, such as a compound that selectively inhibits farnesyltransferase with greater potency (lower IC₅₀ value) relative to the level of inhibition of geranylgeranyl transferase type-1. In a preferred embodiment, the farnesyltransferase inhibitor or selective farnesyltransferase inhibitor administered according to the methods provided herein is the compound of Formula (I), (II) or (III), or a pharmaceutically acceptable form thereof.

In some embodiments, the farnesyltransferase inhibitor or selective farnesyltransferase inhibitor compound used in the methods provided herein is a compound of Formula (I) as shown below, which can be named “(S)-3-amino-3-(1-methyl-1H-imidazol-5-yl)-6-oxa-2 (4,6)-quinolina-1,4 (1,3)-dibenzenacyclohexaphane-2²,4⁴-dicarbonitrile”; also referred to herein as “Compound(S)-1”:

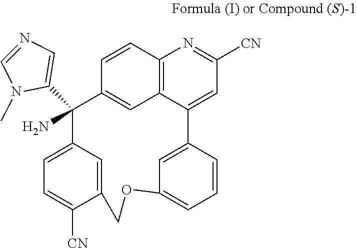 [SMILES: Cn1cncc1[C@@]1(N)c2ccc(C#N)c(c2)COc2cccc(c2)-c2cc(C#N)nc3ccc1cc23]

or a pharmaceutically acceptable form thereof.

In some embodiments, the farnesyltransferase inhibitor or selective farnesyltransferase inhibitor compound used in the methods provided herein is a compound of Formula (II) as shown below, which can be named “(R)-3-amino-3-(1-methyl-1H-imidazol-5-yl)-6-oxa-2 (4,6)-quinolina-1,4 (1,3)-dibenzenacyclohexaphane-2²,4⁴-dicarbonitrile”; also referred to herein as “Compound (R)-2”:

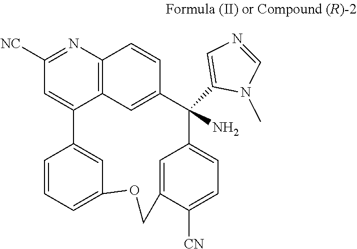 [SMILES: Cn1cncc1[C@]1(N)c2ccc(C#N)c(c2)COc2cccc(c2)-c2cc(C#N)nc3ccc1cc23]

or a pharmaceutically acceptable form thereof.

In some embodiments, the farnesyltransferase inhibitor or selective farnesyltransferase inhibitor compound used in the methods provided herein is a compound of Formula (III) as shown below, which can be named “(3-amino-3-(1-methyl-1H-imidazol-5-yl)-6-oxa-2 (4,6)-quinolina-1,4 (1,3)-dibenzenacyclohexaphane-2²,4⁴-dicarbonitrile”; also referred to herein as “Compound (3)”:

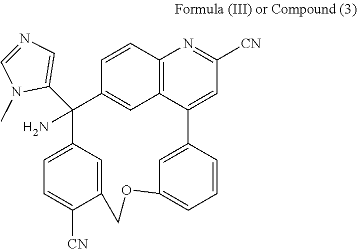 [SMILES: Cn1cncc1C1(N)c2ccc(C#N)c(c2)COc2cccc(c2)-c2cc(C#N)nc3ccc1cc23]

or a pharmaceutically acceptable form thereof.

The synthesis and certain use of the compounds of Formula (I), (II) or (III), or a pharmaceutically acceptable form thereof, as provided herein, are described in U.S. Provisional Application No. 63/285,412, the entirety of which is incorporated herein by reference, and illustrated in Schemes 1 and 2 in Example 1 disclosed herein. The preferred farnesyltransferase inhibitor or selective farnesyltransferase inhibitor administered according to the methods provided herein is a compound of Formula (I), or a pharmaceutically acceptable form thereof. Throughout the instant application, disclosures involving the use of the compound of Formula (I), or pharmaceutically acceptable form thereof (i.e., Compound(S)-1), such disclosures equally apply to the compound of Formula (II), or pharmaceutically acceptable form thereof (i.e., Compound (R)-2), or the compound of Formula (III), or pharmaceutically acceptable form thereof (i.e., Compound (3)).

In some embodiments, the EGFR-TKI used in the methods provided herein is a second, third or fourth generation EGFR TKI. See, e.g., Amelia, T., et al., Molecules 27, 819 (2002); https://doi.org/10.3390/molecules27030819; and He, et al., Eur. J. Med. Chem. 210, 112995 (2021); (https://doi.org/10.1016/j.ejmech.2020.112995). In some embodiments, the EGFR-TKI used in the methods provided herein is an irreversible EGFR-TKI. In some embodiments, the EGFR-TKI is selected from the group comprising, but is not limited to, afatinib (Gilotrif), avitinib (AC0010), BIBW2992, BLU-945, dacomitinib (Vizimpro), EAI045, lazertinib (YH25448/GNS-1480), mavelertinib (PF-06747775; N-((3R,4R)-4-fluoro-1-(6-((3-methoxy-1-methyl-1H-pyrazol-4-yl)amino)-9-methyl-9H-purin-2-yl) pyrrolidine-3-yl) acrylamide), mobocertinib (TAK-788), nazartinib (EGF816), olmutinib (HM61713/BI 1482694), osimertinib (AZD9291), poziotinib (HM781-36B), rociletinib (CO-1686), or tarloxotinib (Tarlox), or a pharmaceutically acceptable form thereof. In some embodiments, the EGFR-TKI used in the methods provided herein is N-(2-{2-dimethylaminoethyl-methylamino}-4-methoxy-5-{[4-(1-methylindol-3-yl)pyrimidin-2-yl]amino}phenyl) prop-2-enamide (herein referred to as osimertinib or AZD9291) or a pharmaceutically acceptable salt thereof. In some embodiments, the preferred pharmaceutically acceptable salt of osimertinib used in the methods provided herein is N-(2-{2-dimethylaminoethyl-methylamino}-4-methoxy-5-{[4-(1-methylindol-3-yl)pyrimidin-2-yl]amino}phenyl) prop-2-enamide mesylate salt (referred to herein as osimertinib mesylate, TAGRISSO®, or Compound 4) of the following formula:

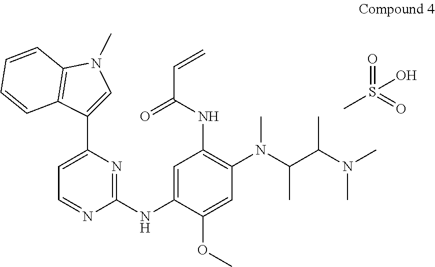

In some embodiments, the EGFR-TKI used in the methods provided herein is a pharmacologically-active metabolite of the EGFR-TKI. For example, in some embodiments, the EGFR-TKI used in the methods provided herein is a pharmacologically-active metabolite of osimertinib, such as AZ7550 or AZ5104.

### 6.2 Pharmaceutical Compositions, Kits and Packaging

In some embodiments, provided herein is a pharmaceutical composition comprising a compound of Formula (I), (II) or (III), or a pharmaceutically acceptable form thereof, and a pharmaceutically acceptable carrier, excipient or diluent.

In some embodiments, provided herein is a pharmaceutical composition comprising an EGFR-TKI, such as osimertinib or a pharmaceutically acceptable salt thereof, such as osimertinib mesylate, and a pharmaceutically acceptable carrier, excipient or diluent.

In some embodiments, provided herein is a pharmaceutical composition comprising a therapeutically effective amount of a compound of Formula (I), (II) or (III) or a pharmaceutically acceptable form thereof, a therapeutically effective amount of an EGFR-TKI such as osimertinib or a pharmaceutically acceptable salt thereof, such as osimertinib mesylate, and a pharmaceutically acceptable carrier, excipient or diluent. For example, in some embodiments, the pharmaceutical composition comprises the compound of Formula (I), or pharmaceutically acceptable form thereof, osimertinib mesylate, and the pharmaceutically acceptable carrier, excipient or diluent.

In some embodiments, provided herein is a pharmaceutical kit comprising (a) a compound of Formula (I), (II) or (III), or a pharmaceutically acceptable form thereof, and (b) an EGFR-TKI such as osimertinib or a pharmaceutically acceptable salt thereof, such as osimertinib mesylate. For example, in some embodiments, the pharmaceutical kit comprises (a) a compound of Formula (I), or a pharmaceutically acceptable form thereof, and (b) osimertinib mesylate. In some embodiments, the pharmaceutical kit further comprises instructions detailing a dosing regimen for administering the compound of Formula (I), or a pharmaceutically acceptable form thereof, and administering the osimertinib mesylate for one or more cycles. In some embodiments, the pharmaceutical kit further comprises a color-coded system that details a dosing regimen for administering the compound of Formula (I), or a pharmaceutically acceptable form thereof, and administering the osimertinib mesylate for one or more cycles. In some embodiments, the pharmaceutical kit is a pharmaceutical packaging.

In some embodiments, the pharmaceutical kit or the pharmaceutical packaging further comprises instructions for administering the contents of the kit to a subject having NSCLC. For example, in some embodiments, the instructions may be color-coded detailing with one color the dosing regimen for administering the compound of Formula (I), or pharmaceutically acceptable form thereof, during a 28-day treating cycle, such as administering once or twice per day on days 1-7, on days 1-7 and 15-21, on days 1-21, or each day of a 28-day treatment cycle, while detailing with a different color the dosing regimen for administering the EGFR-TKI or osimertinib, or pharmaceutically acceptable salt thereof, during a 28-day treating cycle, such as administering once or twice per day on each day of a 28-day treatment cycle. For example, in some embodiments, the instructions may be color-coded detailing an escalation dosing period, a reduction dosing period, or a loading dosing cycle, for administering the compound of Formula (I), or pharmaceutically acceptable form thereof. For example, in some embodiments, the instructions may be color-coded detailing an escalation dosing period or reduction dosing period for administering the EGFR-TKI or osimertinib, or pharmaceutically acceptable salt thereof.

In some embodiments, the pharmaceutical composition, or the pharmaceutical kit or pharmaceutical packaging comprising the same, comprises a therapeutically effective amount of a compound of Formula (I), a pharmaceutically acceptable carrier, excipient or diluent. For example, in some embodiments, the pharmaceutical composition, or the pharmaceutical kit or pharmaceutical packaging comprising the same, comprises 0.5-2400 mg of the compound of Formula (I), or pharmaceutically acceptable form thereof, such as an amount selected from the group consisting of 0.5-2.5 mg, 0.5-5 mg, 0.5-10 mg, 0.5-25 mg, 0.5-50 mg, 0.5-75 mg, 0.5-100 mg, 0.5-300 mg, 0.5-600 mg, 0.5-1200 mg, 1-5 mg, 1-10 mg, 1-25 mg, 1-50 mg, 1-75 mg, 1-100 mg, 1-300 mg, 1-600 mg, 1-1200 mg, 1-2400 mg, 20-100 mg, 40-75 mg, 50-75 mg, 50-100 mg, 50-150 mg, 75-100 mg, 100-200 mg, 125-200 mg, 150-300 mg, 200-250 mg, 200-400 mg, 300-600 mg, 250-500 mg, 400-600 mg, 500-750 mg, 600-900 mg, 700-100 mg, 650-1000 mg, 800-1200 mg, 900-1500 mg, 1000-1600 mg, 1000-2000 mg, 1200-1600 mg, 1500-2000 mg, 1500-2400 mg, 1800-2400 mg and 2000-2400 mg of the compound of Formula (I), or pharmaceutically acceptable form thereof. In some embodiments, the pharmaceutical composition, or the pharmaceutical kit or pharmaceutical packaging comprising the same, comprises about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, about 2050 mg, about 2100 mg, about 2150 mg, about 2200 mg, about 2250 mg, about 2300 mg, about 2350 mg, and about 2400 mg of the compound of Formula (I), or pharmaceutically acceptable form thereof. In some embodiments, the per day dose of a compound of Formula (I), or pharmaceutically acceptable form thereof, is split into two, three, or four doses, such as two, three, or four equal doses, and particularly two doses or two equal doses, that are administered to the subject according to the methods provided herein. In some embodiments, a compound of Formula (I), or pharmaceutically acceptable form thereof, is administered once or twice per day, or is administered once per day, or is administered twice per day.

In some embodiments, the pharmaceutical composition, or the pharmaceutical kit or pharmaceutical packaging comprising the same, comprises 10 to 300 mg of the EGFR-TKI, such as osimertinib or a pharmaceutically acceptable salt thereof, such as an amount selected from the group consisting of 10-30 mg, 10-40 mg, 10-240 mg, 20-50 mg, 20-240 mg, 30-50 mg, 35-70 mg, 40-80 mg, 60-100 mg, 80-120 mg, 80-160 mg, 80-240 mg, 160-250 mg, and 160-300 mg, of the EGFR-TKI, such as osimertinib or a pharmaceutically acceptable salt thereof. In some embodiments, the pharmaceutical composition, or the pharmaceutical kit or pharmaceutical packaging comprising the same, comprises 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, or 300 mg, of the EGFR-TKI, such as osimertinib or a pharmaceutically acceptable salt thereof, such as 40 mg or 80 mg of the EGFR-TKI, such as osimertinib or pharmaceutically acceptable salt thereof. Herein, amounts of the EGFR-TKI are expressed as free base equivalent amounts.

In some embodiments, the pharmaceutical composition, or the pharmaceutical kit or pharmaceutical packaging comprising the same, comprises 10-160 mg of osimertinib or a pharmaceutically acceptable salt thereof, such as an amount selected from the group consisting of 10-30 mg, 10-40 mg, 20-50 mg, 30-50 mg, 35-70 mg, 40-80 mg, 60-100 mg, 80-120 mg, and 80-160 mg of osimertinib or a pharmaceutically acceptable salt thereof. In some embodiments, the pharmaceutical composition, or the pharmaceutical kit or pharmaceutical packaging comprising the same, comprises 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, or 160 mg of osimertinib or a pharmaceutically acceptable salt thereof, such as 40 mg or 80 mg of the osimertinib or pharmaceutically acceptable salt thereof.

In some embodiments, the pharmaceutical composition, or the pharmaceutical kit or pharmaceutical packaging comprising the same, comprising a therapeutically effective amount of the osimertinib comprises 40 mg or 80 mg of osimertinib. In some embodiments, the pharmaceutical composition comprising a therapeutically effective amount of the osimertinib comprises 40 mg of osimertinib. In some embodiments, the pharmaceutical composition comprising a therapeutically effective amount of the osimertinib comprises 80 mg of osimertinib. In some embodiments, the pharmaceutically acceptable salt of osimertinib is osimertinib mesylate. In some embodiments, the pharmaceutical composition comprising a therapeutically effective amount of the osimertinib mesylate, comprises 47.7 mg of osimertinib mesylate. In some embodiments, the pharmaceutical composition comprising a therapeutically effective amount of the osimertinib mesylate, comprises 95.4 mg of osimertinib mesylate.

In some embodiments, the pharmaceutical composition, or the pharmaceutical kit or pharmaceutical packaging comprising the same, comprising the osimertinib or osimertinib mesylate is formulated in a tablet. In some embodiments, the pharmaceutical composition comprising the osimertinib or osimertinib mesylate further comprises an excipient. In some embodiments, the excipient is selected from the group consisting of mannitol, microcrystalline cellulose, low-substituted hydroxypropyl cellulose and sodium stearyl fumarate. In some embodiments, the tablet also consists of polyvinyl alcohol, titanium dioxide, macrogol 3350, talc, ferric oxide yellow, ferric oxide red and ferric oxide black.

In some embodiments, the therapeutically effective amounts of the compound of Formula (I), or pharmaceutically acceptable form thereof, and the osimertinib or pharmaceutically acceptable salt thereof, such as osimertinib mesylate, include amounts effective for treating one or more symptoms of, or amounts effective for treating, retarding progression, mitigating or preventing emergency or delaying the time to emergence of drug resistance, or preventing NSCLC. In some embodiments, therapeutically effective amounts includes amounts effective for reducing or delaying the risk of relapse of NSCLC. In some embodiments, therapeutically effective amounts includes amounts effective for increasing PFS and/or OS. In some embodiments, therapeutically effective amounts includes amounts effective for increasing PFS. In some embodiments, therapeutically effective amounts includes amounts effective for increasing OS. In some embodiments, therapeutically effective amounts includes amounts effective for increasing ORR. In some embodiments, therapeutically effective amounts includes amounts effective for increasing TTP. In some embodiments, therapeutically effective amounts includes amounts effective for increasing PFS therapeutically effective amounts includes amounts effective for increasing EFS. In some embodiments, therapeutically effective amounts includes amounts effective for increasing DoS. In some embodiments, the therapeutically effective amount of the compound of Formula (I), or pharmaceutically acceptable form thereof, and/or the osimertinib or pharmaceutically acceptable salt thereof, such as osimertinib mesylate, in the pharmaceutical composition, or the pharmaceutical kit or pharmaceutical packaging comprising the same, can depend on absorption, tissue distribution, metabolism, excretion rates of the active compound, the dosage schedule, amount administered, particular formulation as well as other factors known to those of skill in the art. The therapeutically effective amount may be determined empirically by testing the compounds in in vitro and in vivo systems described herein and then extrapolated therefrom for dosages for humans.

In some embodiments, the pharmaceutical compositions provided herein comprise a pharmaceutically acceptable carrier, excipient or diluent in an amount sufficient to exert a therapeutically useful effect in the absence of undesirable side effects on the patient treated.

In some embodiments, the pharmaceutical compositions are provided for administration to a subject in unit dosage forms, such as tablets, capsules, microcapsules, pills, powders, granules, troches, suppositories, injections, syrups, patches, creams, lotions, ointments, gels, sprays, sterile parenteral solutions or suspensions, and oral solutions or suspensions, and oil water emulsions containing suitable quantities of the compounds or pharmaceutically acceptable salts thereof. In some embodiments, the pharmaceutical compositions provided herein are in the form of a tablet, In some embodiments, the pharmaceutical compositions provided herein are in the form of a capsule, In some embodiments, the capsules contain a compound provided herein without an additional carrier, excipient or vehicle. Typically the compound disclosed herein is formulated into pharmaceutical compositions using techniques and procedures well known in the art (see, e.g., Ansel Introduction to Pharmaceutical Dosage Forms, Seventh Edition 1999). In some embodiments, the pharmaceutical compositions are formulated and administered in unit dosage forms or multiple dosage forms. Such dosage forms contain predetermined amounts of active ingredients, and may be prepared by methods of pharmacy well known to those skilled in the art. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

The pharmaceutical compositions provided herein may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens may be adjusted over time according to the individual need and the professional judgment of the person administering or supervising the administration of the pharmaceutical compositions, and that the concentration ranges set forth herein are exemplary only and are not intended to limit the scope or practice of the claimed pharmaceutical compositions.

The pharmaceutical compositions are intended to be administered by a suitable route, including but not limited to orally, parenterally, rectally, topically and locally. For oral administration, capsules and tablets can be formulated. The pharmaceutical compositions are in liquid, semi-liquid or solid form and are formulated in a manner suitable for each route of administration. In one embodiment, when administered orally, a compound provided herein is administered with a meal and water. In another embodiment, the compound provided herein is dispersed in water or juice (e.g., apple juice or orange juice) and administered orally as a solution or a suspension. In one embodiment, a compound provided herein is administered when the subject is fed. In one embodiment, a compound provided herein is administered when the subject is fed with high-fat and/or high-calorie food. In one embodiment, a compound provided herein is administered when the subject is fed with FDA-standard high-fat high-calorie breakfast. In one embodiment, a compound provided herein is administered when the subject is fasted. In one embodiment, a compound provided herein is administered after the subject has an at least 8-hour overnight fast. In one embodiment, a compound provided herein is administered with or without food.

The pharmaceutical compositions provided herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition. Depending on the state of the disease to be treated and the subject's condition, a composition may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. The compound of Formula (I), (II) or (III), or a pharmaceutically acceptable form thereof, and/or the osimertinib or a pharmaceutically acceptable salt thereof, such as osimertinib mesylate, may be formulated, alone or together, in suitable dosage unit with pharmaceutically acceptable excipients, carriers, adjuvants and vehicles, appropriate for each route of administration.

In some embodiments, the pharmacokinetic profile of pharmaceutical compositions provided herein can be delayed or prolonged by proper formulation. For example, a slowly soluble pellet of the compound provided herein can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Parenteral preparations can be made long-acting, by dissolving or suspending the compound provided herein in oily or emulsified vehicles that allow it to disperse slowly in the serum.

### 6.3 Methods, Dosing Regimens and Schedules

### 6.3.1 Therapeutic Methods

In some embodiments, provided herein is a method of treating non-small cell lung cancer (NSCLC) in a subject, comprising administering to the subject (a) a compound of Formula (I), (II) or (III), or a pharmaceutically acceptable form thereof, and (b) an EGFR-TKI. In some embodiments, the method of treating provided herein comprises administering to the subject (a) a therapeutically effective amount of a compound of Formula (I):

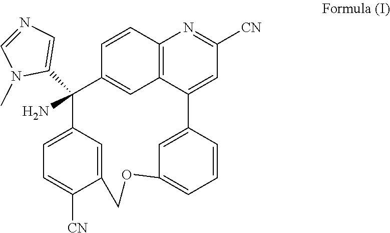 [SMILES: Cn1cncc1[C@@]1(N)c2ccc(C#N)c(c2)COc2cccc(c2)-c2cc(C#N)nc3ccc1cc23]

or a pharmaceutically acceptable form thereof, and (b) a therapeutically effective amount of an EGFR-TKI. In some embodiments, the method of treating provided herein comprises administering to the subject (a) a pharmaceutical composition comprising a therapeutically effective amount of a compound of Formula (I), or a pharmaceutically acceptable form thereof, and a pharmaceutically acceptable carrier, excipient or diluent, and (b) a therapeutically effective amount of an EGFR-TKI, and a pharmaceutically acceptable carrier, excipient or diluent. In some embodiments, the EGFR-TKI is an irreversible EGFR-TKI. In some embodiments, the EGFR-TKI is osimertinib or a pharmaceutically acceptable salt thereof, such as osimertinib mesylate. In some embodiments, the method of treating provided herein comprises administering the compound of Formula (I), or pharmaceutically acceptable form thereof, before, after, or simultaneously with the osimertinib, or pharmaceutically acceptable salt thereof, during one or more cycles. In some embodiments, the subject treated according to the methods of treating provided herein has, suffers from, has symptoms associated with, or is diagnosed as having, NSCLC. In some embodiments, provided herein is a method of treating EGFR-mediated non-small cell lung cancer (NSCLC) in a subject, comprising administering to the subject a compound of Formula (I) or a pharmaceutically acceptable form thereof, or a pharmaceutical composition comprising a compound of Formula (I) or pharmaceutically acceptable form thereof. In some embodiments, the NSCLC is EGFR-mutant NSCLC.

In some embodiments, the method of treating provided herein comprises administering to the subject a pharmaceutical kit or pharmaceutical packaging comprising (a) a pharmaceutical composition comprising a therapeutically effective amount of a compound of Formula (I), or a pharmaceutically acceptable form thereof, and a pharmaceutically acceptable carrier, excipient or diluent, and for combination methods, (b) a pharmaceutical composition comprising a therapeutically effective amount of an EGFR-TKI, and a pharmaceutically acceptable carrier, excipient or diluent. In some embodiments, the pharmaceutical kit or pharmaceutical packaging further comprise instructions detailing the dosing regimen for administering the compound of Formula (I), or pharmaceutically acceptable form thereof, and for combination methods, the EGFR-TKI, for one or more cycles. In some embodiments, the EGFR-TKI is an irreversible EGFR-TKI. In some embodiments, the EGFR-TKI is osimertinib or a pharmaceutically acceptable salt thereof, such as osimertinib mesylate. In some embodiments, the method of treating provided herein comprises administering the compound of Formula (I), or pharmaceutically acceptable form thereof, before, after, or simultaneously with, the osimertinib, or pharmaceutically acceptable salt thereof, during one or more cycles. In some embodiments, the subject treated according to the methods of treating provided herein has, suffers from, has symptoms associated with, or is diagnosed as having, NSCLC.

In some embodiments, the method of treating includes mitigating EGFR-TKI resistance in NSCLC subject currently or previously treated with an EGFR-TKI, comprising administering to the NSCLC subject (a) a compound of Formula (I), or a pharmaceutically acceptable form thereof, or pharmaceutically acceptable form thereof (or a pharmaceutical composition comprising the same), alone or in combination with an EGFR-TKI treatment. For example, in some embodiments, provided herein is a method of mitigating osimertinib resistance in an osimertinib-resistant NSCLC subject, comprising administering to the subject (a) a compound of Formula (I), or a pharmaceutically acceptable form thereof (or a pharmaceutical composition comprising the same), and (b) osimertinib, or a pharmaceutically acceptable salt thereof (or a pharmaceutical composition comprising the same), wherein subject is currently being treated or was previously treated with osimertinib.

In some embodiments, the method of treating includes preventing or delaying emergence of EGFR-TKI resistance in an EGFR-TKI-naïve NSCLC subject, comprising administering to the subject (a) a compound of Formula (I), or a pharmaceutically acceptable form thereof (or a pharmaceutical composition comprising the same), before, during or after administering an EGFR-TKI. For example, in some embodiments, provided herein is a method of preventing or delaying emergence of osimertinib resistance in an osimertinib-naïve NSCLC subject, comprising administering to the subject (a) a compound of Formula (I), or a pharmaceutically acceptable form thereof (or a pharmaceutical composition comprising the same), before, during or after administering osimertinib (or a pharmaceutical composition comprising the same).

In some embodiments, the subject treated according to the methods of treating provided herein has, suffers from, has symptoms associated with, or is diagnosed as having, NSCLC. In some embodiments, the subject has or suffers from NSCLC. In some embodiments, the subject has symptoms associated with NSCLC. In some embodiments, the subject is diagnosed as having NSCLC. In some embodiments, the subject is a previously treated NSCLC subject. In some embodiments, the subject is an EGFR-TKI naïve subject. In some embodiments, the subject is an EGFR-TKI naïve subject for NSCLC. In some embodiments, the subject is an osimertinib naïve subject. In some embodiments, the subject is an osimertinib-naïve subject for NSCLC. In some embodiments, the subject has been treated previously with an EGFR-TKI. In some embodiments, the subject has been treated previously with osimertinib. In some embodiments, the subject is a NSCLC subject in remission. In some embodiments, the NSCLC subject is an EGFR-TKI resistant NSCLC subject, such as an osimertinib-resistant NSCLC subject. In some embodiments, the subject is a mammal, for example, a human, such as a human having, suffering from, having symptoms associated with, or diagnosed as having, NSCLC. In some embodiments, the NSCLC has squamous histology.

In some embodiments, the EGFR-TKI used according to the methods provided herein is a second, third or fourth generation EGFR TKI. In some embodiments, the EGFR-TKI used in the methods provided herein is an irreversible EGFR-TKI. In some embodiments, the EGFR-TKI is selected from the group comprising, but is not limited to, afatinib (Gilotrif), avitinib (AC0010), BIBW2992, BLU-945, dacomitinib (Vizimpro), EAI045, lazertinib (YH25448/GNS-1480), mavelertinib (PF-06747775; N-((3R,4R)-4-fluoro-1-(6-((3-methoxy-1-methyl-1H-pyrazol-4-yl)amino)-9-methyl-9H-purin-2-yl) pyrrolidine-3-yl) acrylamide), mobocertinib (TAK-788), nazartinib (EGF816), olmutinib (HM61713/BI 1482694), osimertinib (AZD9291), poziotinib (HM781-36B), rociletinib (CO-1686), or tarloxotinib (Tarlox), or a pharmaceutically acceptable form thereof. In some embodiments, the EGFR-TKI is osimertinib or a pharmaceutically acceptable form thereof, such as osimertinib mesylate.

In some embodiments, the subject treated according to the methods provided herein has early stage NSCLC, metastatic NSCLC, advanced NSCLC, relapsed NSCLC, or refractory NSCLC. In some embodiments, the subject has early stage NSCLC. In some embodiments, the subject has metastatic NSCLC or advanced NSCLC. In some embodiments, the subject has relapsed NSCLC or refractory NSCLC. In some embodiments, the subject has an EGFR-mediated NSCLC. In some embodiments, the NSCLC is a solid tumor. In some embodiments, the NSCLC is an EGFR-mediated NSCLC. In some embodiments, the NSCLC is an EGFR-mutant mediated NSCLC. In some embodiments, the subject is diagnosed as having EGFR-mutant mediated NSCLC. In some embodiments, the subject may be diagnosed as having an EGFR-mutant mediated NSCLC by detection and analysis with an FDA-approved test, such as a Cobas® EGFR Mutation Test. In some embodiments, the EGFR-mutant mediated NSCLC is selected from the group consisting of: an exon 19 deletion, an exon 21 L858R mutation, a T790M mutation, an exon 20 insertion, and a C797S mutation. For example, in some embodiments, the EGFR-mutant mediated NSCLC has two or more, or three or more, EGFR mutations selected from the group consisting of an exon 19 deletion, an exon 21 L858R mutation, a T790M mutation, an exon 20 insertion, and a C797S mutation in EGFR. In some embodiments, the EGFR-mutant mediated NSCLC is or comprises an exon 19 deletion. In some embodiments, the EGFR-mutant mediated NSCLC is or comprises an exon 21 L858R mutation. In some embodiments, the EGFR-mutant mediated NSCLC is or comprises a T790M mutation. In some embodiments, the EGFR-mutant mediated NSCLC is or comprises an exon 20 insertion. In some embodiments, the EGFR-mutant mediated NSCLC is or comprises a C797S mutation. In some embodiments, the subject is a mammal, for example, a human, such as a human having, suffering from, having symptoms associated with, or diagnosed as having, NSCLC. In some embodiments, the exon 20 insertion mutation is a post C-helix insertion of 1 to 4 amino acids. These mutations account for 80-90% of all ex20ins mutations. In some embodiments, the exon 20 insertion mutation is A763_Y764insFQEA, A763_Y764insFQEA, Y764_V765insHH, M766_A767insAI, V769_D770insASV, D770_N771insNPG, D770_N771insNPG, D770_N77lins_SSVD, or H773 (see Hou et al., Biomark. Res. 10, 21 (2022)). In some embodiments, the subject has an exon 20 insertion mutation in EGFR and at least one other EGFR mutation.

In some embodiments, the compound of Formula (I), or pharmaceutically acceptable form thereof, administered according to the methods provided herein inhibits farnesylation of a protein, for example inhibits farnesylation of a farnesylation-dependent protein. Without being bound by any one theory, in some embodiments, the compound of Formula (I), or pharmaceutically acceptable form thereof, administered according to the methods provided herein inhibits farnesylation of a farnesylation-dependent protein selected from the group consisting of RhoB, RhoE and Lamin B. In some embodiments, the compound of Formula (I), or pharmaceutically acceptable form thereof, administered according to the methods provided herein inhibits farnesylation of a dysregulated farnesylation-dependent protein selected from the group consisting of RhoB, RhoE and Lamin B. In some embodiments, the compound of Formula (I), or pharmaceutically acceptable form thereof, inhibits the farnesylation of RhoB, such as dysregulated RhoB. In some embodiments, the compound of Formula (I), or pharmaceutically acceptable form thereof, inhibits the farnesylation of RhoE, such as dysregulated RhoE. In some embodiments, the compound of Formula (I), or pharmaceutically acceptable form thereof, inhibits the farnesylation of Lamin B, such as dysregulated Lamin B.

In some embodiments, inhibition the farnesylation of the farnesylation-dependent protein, according to the methods provided herein, occurs in a cell, such as in a cell of the subject. In some embodiments, the cell is a mammalian cell. In some embodiments, the cell a human cell. Without being bound by any one theory, in some embodiments, inhibiting farnesylation of the farnesylation-dependent protein by administering the compound of Formula (I), or pharmaceutically acceptable form thereof, according to the methods provided herein, provides a synergistic benefit, such as a therapeutic benefit to the subject. For example, in some embodiments, the synergistic benefit or therapeutic benefit provided by administering the compound of Formula (I), or pharmaceutically acceptable form thereof, according to the methods provided herein, includes, but is not limited to, improving efficacy (e.g., suppressing tumor growth and inducing tumor regression); increasing PFS and/or OS, such as increasing PFS 2 fold, 3 fold, or 4 fold, or increasing OS 2 fold, 3 fold, or 4 fold; reducing the therapeutically effective amount of the osimertinib, or pharmaceutically acceptable salt thereof, reducing osimertinib-associated toxicity, such as reducing a toxicity selected from the group consisting of leukopenia, lymphopenia, thrombocytopenia, diarrhea, anemia, rash, musculoskeletal pain, nail toxicity, neutropenia, dry skin, stomatitis, fatigue, and cough; or delaying emergence of osimertinib resistance, such as unexpectedly delaying emergence of osimertinib resistance. In some embodiments, the inhibition of the farnesyltransferase present in the cell takes place in a subject suffering from NSCLC. In some embodiments, the NSCLC is selected from the group consisting of: an early stage NSCLC, an advanced NSCLC, a metastatic NSCLC, a relapsed NSCLC, and a refractory NSCLC. In some embodiments, the NSCLC is an EGFR-mediated NSCLC. In some embodiments, the NSCLC is an EGFR-mutant mediated NSCLC. In some embodiments, the NSCLC has an EGFR mutation. In some embodiments, the NSCLC has an EGFR mutation selected from the group consisting of: an exon 19 deletion, an exon 21 L858R mutation, a T790M mutation, an exon 20 insertion, and a C797S mutation. In some embodiments, the subject is a mammal, for example, a human, such as a human having, suffering from, having symptoms associated with, or diagnosed as having, NSCLC. In some embodiments, the NSCLC has squamous histology.

In some embodiments, the methods provided herein provide one or more synergistic or therapeutic benefits to the subject. In some embodiments, the therapeutic benefit may be measured relative to treatment with an FTI or an EGFR-TKI alone, or relative to standard of care for the indication (such as surgery, radiofrequency ablation, radiation therapy, or chemotherapy, or a combination thereof), or relative to no treatment for an advanced tumor. For example, in some embodiments, the methods provided herein delay the time to emergence of drug resistance or delays progression of drug resistance, for example, EGFR-TKI drug resistance, such as osimertinib resistance. In some embodiments, the methods provided herein delay, halt or prevent progression of NSCLC. In some embodiments, the methods provided herein delay, halt or prevent NSCLC tumor growth. In some embodiments, one or more therapeutic benefits provided by the methods disclosed herein includes a reduction of a primary tumor, such as a reduction in the size, volume, or appearance of a primary tumor or a reduction in the extent of metastasis from a primary tumor. In some embodiments, the methods provided herein reduce a primary NSCLC tumor. In some embodiments, the methods provided herein provide relief of NSCLC tumor-related symptoms. In some embodiments, the methods provided herein inhibit NSCLC tumor-secreted factors. In some embodiments, the methods provided herein delay the appearance of primary or secondary NSCLC tumors. In some embodiments, the methods provided herein slow the development of primary or secondary NSCLC tumors. In some embodiments, the methods provided herein decrease the occurrence of primary or secondary NSCLC tumors. In some embodiments, the methods provided herein slow or decrease the severity of secondary effects associated with NSCLC. In some embodiments, the methods provided herein arrest NSCLC tumor growth and reduce NSCLC tumors. In some embodiments, the methods provided herein increase Time To Progression (TTP), Progression Free Survival (PFS), Event-free survival (EFS), Overall Survival (OS), overall response rate (ORR), or duration of response (DoR), or a combinations thereof. In some embodiments, the methods provided herein increase TTP. In some embodiments, the methods provided herein increase PFS. In some embodiments, the methods provided herein increase EFS. In some embodiments, the methods provided herein increase OS. In some embodiments, the methods provided herein increase ORR. In some embodiments, the methods provided herein increase DoR. In some embodiments, the methods provided herein decrease time to response (TTR).

In some embodiments, the methods provided herein can be for second line therapy, third line therapy, second or greater line therapy, or third or greater line therapy. In such cases, a subject may have received prior treatment selected from: chemotherapy or a TKI such as osimertinib, wherein the methods provide one or more therapeutic benefits to the subject, (a) for combinations with an EGFR-TKI, relative to treatment with EGFR-TKI monotherapy, such as relative to treatment an EGFR-TKI that eventually leads to relapse and/or resistance in the subject, for example, relative to treatment with osimertinib; (b) relative to standard of care advanced solid tumor treatment, including but not limited to surgery, radiofrequency ablation, radiation therapy, or chemotherapy, or combinations thereof; or (c) relative to no treatment. In some embodiments, the methods provided herein include one or more prior treatments according to the NCCN Guidelines.

In some embodiments, the compounds or pharmaceutical compositions provided herein are administered according to methods known in the art. For example, in some embodiments, the compounds or pharmaceutical compositions provided herein are administered orally, intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. For example, in some embodiments, the compound of Formula (I), or pharmaceutically acceptable form thereof, is administered to the subject orally. For example, in some embodiments, osimertinib, or a pharmaceutically acceptable salt thereof, such as osimertinib mesylate, is administered to the subject orally.

### 6.3.2 Doses and Regimens

Provided herein are methods comprising administering to a subject with NSCLC (a) a compound of Formula (I), or a pharmaceutically acceptable form thereof, and (b) an EGFR-TKI such as osimertinib, or a pharmaceutically acceptable salt thereof, such as osimertinib mesylate. In some embodiments, the method comprises administering to the subject (a) a therapeutically effective amount of a compound of Formula (I), or a pharmaceutically acceptable form thereof, and (b) a therapeutically effective amount of an EGFR-TKI such as osimertinib, or a pharmaceutically acceptable salt thereof, such as osimertinib mesylate. In some embodiments, the method comprises administering to the subject (a) a pharmaceutical composition comprising a therapeutically effective amount of a compound of Formula (I), or a pharmaceutically acceptable form thereof, and a pharmaceutically acceptable carrier, excipient or diluent, and (b) a therapeutically effective amount of an EGFR-TKI such as osimertinib, or a pharmaceutically acceptable salt thereof, such as osimertinib mesylate, and a pharmaceutically acceptable carrier, excipient or diluent. In some embodiments, the method of treating provided herein comprises administering to the subject a compound of Formula (I) or a pharmaceutically acceptable form thereof. In some embodiments are provided methods of mitigating, delaying emergence of, or preventing emergence of EGFR-TKI or osimertinib resistance in an NSCLC subject comprising administering to the subject a therapeutically effective amount of the compound of Formula (I) or a pharmaceutically acceptable form thereof and a therapeutically effective amount of an EGFRI-TKI such as osimertinib, or a pharmaceutically acceptable salt thereof. In some embodiments are provided methods of treating EGFR-mediated NSCLC in a subject comprising administering to the subject a therapeutically effective amount of a compound of Formula (I) or a pharmaceutically acceptable form thereof.

In some embodiments, the compound of Formula (I), or pharmaceutically acceptable form thereof, is administered to the subject according to the methods provided herein at a dose of 0.5-2400 mg per day. In some embodiments, the dose of the compound of Formula (I), or pharmaceutically acceptable form thereof, is selected from the group consisting of 0.5-2.5 mg, 0.5-5 mg, 0.5-10 mg, 0.5-25 mg, 0.5-50 mg, 0.5-75 mg, 0.5-100 mg, 0.5-300 mg, 0.5-600 mg, 0.5-1200 mg, 1-5 mg, 1-10 mg, 1-25 mg, 1-50 mg, 1-75 mg, 1-100 mg, 1-300 mg, 1-600 mg, 1-1200 mg, 1-2400 mg, 20-100 mg, 40-75 mg, 50-75 mg, 50-100 mg, 50-150 mg, 75-100 mg, 100-200 mg, 125-200 mg, 150-300 mg, 200-250 mg, 200-400 mg, 300-600 mg, 250-500 mg, 400-600 mg, 500-750 mg, 600-900 mg, 700-100 mg, 650-1000 mg, 800-1200 mg, 900-1500 mg, 1000-1600 mg, 1000-2000 mg, 1200-1600 mg, 1500-2000 mg, 1500-2400 mg, 1800-2400 mg and 2000-2400 mg per day. In some embodiments, the dose of the compound of Formula (I), or pharmaceutically acceptable form thereof, is selected from the group consisting of about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.5 mg, about 3.0 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, about 2050 mg, about 2100 mg, about 2150 mg, about 2200 mg, about 2250 mg, about 2300 mg, about 2350 mg, and about 2400 mg per day. In some embodiments, the dose of the compound of Formula (I), or pharmaceutically acceptable form thereof, is administered 1, 2, 3, or 4 times per day. In some embodiments, the per day dose of the compound of Formula (I), or pharmaceutically acceptable form thereof, is split into two, three, or four doses, such as two, three, or four equal doses, and particularly two doses or two equal doses, that are administered to the subject according to the methods provided herein. In some embodiments, the dose of the compound of Formula (I), or pharmaceutically acceptable form thereof, is administered once or twice per day, such as once per day.

In some embodiments, the compound of Formula (I), or pharmaceutically acceptable form thereof, is administered to the subject according to the methods provided herein at a dose of 0.01-50 mg/kg body weight per day. In some embodiments, the dose of the compound of Formula (I), or pharmaceutically acceptable form thereof, is selected from the group consisting of 0.01-1 mg/kg, 0.01-2.5 mg/kg, 0.01-5 mg/kg, 0.1-5 mg/kg, 0.1-10 mg/kg, 0.1-20 mg/kg, 1-30 mg/kg, 1-40 mg/kg, 5-50 mg/kg, 10-50 mg/kg, 15-50 mg/kg, 20-50 mg/kg, 25-50 mg/kg, 30-50 mg/kg, 40-50 mg/kg, 20-40 mg/kg, and 25-25 mg/kg body weight per day. In some embodiments, the dose of the compound of Formula (I), or pharmaceutically acceptable form thereof, is selected from the group consisting of about 0.01 mg/kg, about 0.02 mg/kg, about 0.05 mg/kg, about 0.06 mg/kg, about 0.07 mg/kg, about 0.08 mg/kg, about 0.09 mg/kg, about 0.1. 41-mg/kg, about 0.5 mg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, and about 50 mg/kg body weight per day. In some embodiments, the dose of the compound of Formula (I), or pharmaceutically acceptable form thereof, is administered 1, 2, 3, or 4 times per day, for example, is administered once or twice per day, such as once per day.

In some embodiments, the dose of the compound of Formula (I), or pharmaceutically acceptable form thereof, is administered to the subject monthly, weekly, or daily, according to the methods provided herein. In some embodiments, the dose of the compound of Formula (I), or pharmaceutically acceptable form thereof, is administered to the subject 1, 2, 3 or 4 times per day for one or more cycles. In some embodiments, the dose of the compound of Formula (I), or pharmaceutically acceptable form thereof, is split into two doses that are administered to the subject according to the methods provided herein. In some embodiments, the dose of the compound of Formula (I), or pharmaceutically acceptable form thereof, is administered once or twice per day for one or more cycles, such as once per day for one or more cycles. In some embodiments, the dose of the compound of Formula (I), or pharmaceutically acceptable form thereof, is administered to the subject 1, 2, 3 or 4 times per day continuously for unlimited days or until remission achieved in said subject. In some embodiments, the compound of Formula (I), or pharmaceutically acceptable salt thereof, is administered to the subject once per day (sometimes referred to as QD) for one or more cycles, such as for two or more cycles, three or more cycles, or four or more cycles. In some embodiments, the compound of Formula (I), or pharmaceutically acceptable salt thereof, is administered to the subject twice per day (sometimes referred to as BID) for one or more cycles, such as for two or more cycles, three or more cycles, or four or more cycles. In some embodiments, the cycle (sometimes referred to herein as a treating cycle or maintenance cycle) is 1 day, 7 days or 28 days. In some embodiments, the cycle is 1 day. In some embodiments, the cycle is 7 days. In some embodiments, the cycle is 28 days. In some embodiments, the 28-day cycle is preferred. In some embodiments, the compound of Formula (I), or pharmaceutically acceptable salt thereof, is administered to the subject once per day for one or more 28-day cycles. In some embodiments, the compound of Formula (I), or pharmaceutically acceptable salt thereof, is administered to the subject twice per day for one or more 28-day cycles. In some embodiments, the compound of Formula (I), or pharmaceutically acceptable salt thereof, is administered to the subject once or twice per day every other week during a 28-day cycle.

In some embodiments, the compound of Formula (I), or pharmaceutically acceptable form thereof, is administered to the subject 1, 2, 3 or 4 times per day on days 1-7, days 1-7 and 15-21, days 1-21, or each day (i.e., days 1-28) of a 28-day treatment cycle, for one of more cycles, according to the methods provided herein. For example, in some embodiments, the compound of Formula (I), or pharmaceutically acceptable form thereof, is administered to the subject QD on days 1-7, days 1-7 and 15-21, days 1-21, or each day (i.e., days 1-28) of a 28-day treatment cycle, for one of more cycles. For example, in some embodiments, the compound of Formula (I), or pharmaceutically acceptable form thereof, is administered to the subject BID on days 1-7, days 1-7 and 15-21, days 1-21, or each day (i.e., days 1-28) of a 28-day treatment cycle, for one of more cycles. In some embodiments, the compound of Formula (I), or pharmaceutically acceptable form thereof, is administered to the subject on QD on days 1-7 of a 28-day treatment cycle, for one of more cycles. In some embodiments, the compound of Formula (I), or pharmaceutically acceptable form thereof, is administered to the subject BID on days 1-7 of a 28-day treatment cycle, for one of more cycles. In some embodiments, the compound of Formula (I), or pharmaceutically acceptable form thereof, is administered to the subject QD on days 1-7 and 15-21 of a 28-day treatment cycle, for one of more cycles. In some embodiments, the compound of Formula (I), or pharmaceutically acceptable form thereof, is administered to the subject BID on days 1-7 and 15-21 of a 28-day treatment cycle, for one of more cycles. In some embodiments, the compound of Formula (I), or pharmaceutically acceptable form thereof, is administered to the subject QD on days 1-21 of a 28-day treatment cycle, for one of more cycles. In some embodiments, the compound of Formula (I), or pharmaceutically acceptable form thereof, is administered to the subject BID on days 1-21 of a 28-day treatment cycle, for one of more cycles. In some embodiments, the compound of Formula (I), or pharmaceutically acceptable form thereof, is administered to the subject QD on each day (i.e., days 1-28) of a 28-day treatment cycle, for one of more cycles. In some embodiments, the compound of Formula (I), or pharmaceutically acceptable form thereof, is administered to the subject BID on each day (i.e., days 1-28) of a 28-day treatment cycle, for one of more cycles.

In some embodiments, the EGFR-TKI, preferably osimertinib or pharmaceutically acceptable salt thereof, such as osimertinib mesylate, is administered to the subject according to the methods provided herein at a dose of 10-160 mg per day. In some embodiments, the dose of the EGFR-TKI, such as osimertinib or pharmaceutically acceptable salt thereof, administered to the subject is selected from the group consisting of 10-30 mg, 10-40 mg, 20-50 mg, 30-50 mg, 35-70 mg, 40-80 mg, 60-100 mg, 80-120 mg, and 80-160 mg per day. In some embodiments, the osimertinib or a pharmaceutically acceptable salt thereof, is administered to the subject according to the methods provided herein at a dose selected from the group consisting of about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, and about 160 mg per day. In some embodiments, the dose of the osimertinib or pharmaceutically acceptable salt thereof, administered to the subject is 20 mg, 40 mg, 50 mg, 60 mg, 70 mg, or 80 mg per day, such as 40 mg or 80 mg per day. In some embodiments, the dose of the EGFR-TKI, preferably osimertinib or pharmaceutically acceptable form thereof, is administered 1, 2, 3, or 4 times per day. In some embodiments, the dose of the osimertinib or pharmaceutically acceptable form thereof is split into two doses that are administered to the subject according to the methods provided herein. In some embodiments, the dose of the osimertinib or pharmaceutically acceptable form thereof is administered once or twice per day, such as once per day. In some embodiments, the pharmaceutically acceptable salt of osimertinib is osimertinib mesylate.

In some embodiments, the dose of the osimertinib or pharmaceutically acceptable salt thereof, is administered to the subject daily for one or more cycles according to the methods provided herein. For example, in some embodiments, the dose of the osimertinib or pharmaceutically acceptable salt thereof is administered 1, 2, 3 or 4 times per day for one or more cycles. In some embodiments, the dose of the osimertinib or pharmaceutically acceptable salt thereof is split into two doses that are administered to the subject according to the methods provided herein. In some embodiments, the dose of the osimertinib or pharmaceutically acceptable salt thereof is administered once or twice per day for one or more cycles, such as once per day for one or more cycles. In some embodiments, the dose of the osimertinib or pharmaceutically acceptable salt thereof, is administered to the subject 1, 2, 3 or 4 times per day continuously for unlimited days or until remission achieved in said subject. In some embodiments, the dose of the osimertinib or pharmaceutically acceptable salt thereof, is administered to the subject once per day (sometimes referred to as QD) for one or more cycles, such as for two or more cycles, three or more cycles, or four or more cycles. In some embodiments, the dose of the osimertinib or pharmaceutically acceptable salt thereof, is split into two doses that are administered to the subject according to the methods provided herein. For example, in some embodiments, the dose of the osimertinib or pharmaceutically acceptable salt thereof, is administered to the subject twice per day (sometimes referred to as BID) for one or more cycles, such as for two or more cycles, three or more cycles, or four or more cycles. In some embodiments, the cycle (e.g., a treating cycle or maintenance cycle) is 1 day, 7 days or 28 days. In some embodiments, the cycle is 1 day. In some embodiments, the cycle is 7 days. In some embodiments, the cycle is 28 days. In some embodiments, the 28-day cycle is preferred. In some embodiments, the dose of the osimertinib or pharmaceutically acceptable salt thereof, is administered to the subject once per day for one or more 28-day cycles. In some embodiments, the dose of the osimertinib or pharmaceutically acceptable salt thereof, is administered to the subject twice per day for one or more 28-day cycles. In some embodiments, the dose of the osimertinib or pharmaceutically acceptable salt thereof, is administered to the subject once or twice per day every other week during a 28-day cycle. In some embodiments, the pharmaceutically acceptable salt of osimertinib is administered. In some embodiments, the pharmaceutically acceptable salt of osimertinib is osimertinib mesylate.

In some embodiments, the methods provided herein comprise (1) an escalating dosing cycle, followed by (2) one or more treating cycles (sometimes referred to as maintenance cycles). In some embodiments, the methods provided herein comprise (1) an escalating dosing cycle, comprising administering (a) escalating daily doses of a compound of Formula (I), or a pharmaceutically acceptable form thereof, and (b) a dose of osimertinib, or a pharmaceutically acceptable form thereof, followed by (2) one or more treating cycles (sometimes referred to as maintenance cycles), comprising administering (a) a dose of the compound of Formula (I), or pharmaceutically acceptable form thereof, and (b) a dose of the osimertinib, or pharmaceutically acceptable form thereof. In some embodiments, the compound of Formula (I), or pharmaceutically acceptable form thereof, is administered 1, 2, 3, or 4 times per day during the escalating dosing cycle, for example, once or twice per day. In some embodiments, the escalating dosing cycle is for 1 day, 2 days, 3 days, 4 days, 5 days, 6 days, 7 days, 14 days, 21 days, or 28 days. In some embodiments, inclusion of an escalating dosing cycle provides a synergistic or therapeutic benefit to the subject, including but not limited to, identifying a therapeutically effective dose for the subject, improving the efficacy of the osimertinib, mitigating or avoiding toxicities, adverse events or adverse symptoms, or combinations thereof, associated with the compound of Formula (I), or pharmaceutically acceptable form thereof, or associated with osimertinib, or pharmaceutically acceptable form thereof. In some embodiments, the pharmaceutically acceptable salt of osimertinib is osimertinib mesylate. In some embodiments, the methods provided herein comprise (1) a loading dosing cycle, followed by (2) one or more treating cycles (sometimes referred to as maintenance cycles). In some embodiments, the methods provided herein comprise (1) a loading dosing cycle, comprising administering (a) a loading dose of a compound of Formula (I), or a pharmaceutically acceptable form thereof, and (b) a dose of osimertinib, or a pharmaceutically acceptable form thereof, followed by (2) one or more treating cycles (sometimes referred to as maintenance cycles), comprising administering (a) a dose of the compound of Formula (I), or pharmaceutically acceptable form thereof, and (b) a dose of the osimertinib, or pharmaceutically acceptable form thereof. In some embodiments, the loading dose (sometimes referred to as an elevated dose or a bolus dose) of the compound of Formula (I), or pharmaceutically acceptable form thereof, is 1.1-10 times the dose administered during the one or more treating cycles. For example, in some embodiments, the loading dose of the compound of Formula (I), or pharmaceutically acceptable form thereof, is 1.1, 1.2, 1.3, 1.4, 1.5, 1.6, 1.7, 1.8, 1.9, 2, 3, 4, 5, 6, 7, 8, 9, or 10 times the dose administered during the one or more treating cycles. In some embodiments, administration of the compound of Formula (I), or pharmaceutically acceptable form thereof, during the loading dosing cycle is 1, 2, 3, or 4 times per day. In some embodiments, administration of the compound of Formula (I), or pharmaceutically acceptable form thereof, during the loading dosing cycle is once per day. In some embodiments, administration of the compound of Formula (I), or pharmaceutically acceptable form thereof, during the loading dosing cycle is twice per day. In some embodiments, the loading dosing cycle is for 1 day, 2 days, 3 days, 4 days, 5 days, 6 days, 7 days, 14 days, 21 days, or 28 days. In some embodiments, inclusion of a loading dosing cycle prior to treating the subject with one or more treating or maintenance cycles, provides a synergistic or therapeutic benefit to the subject, including but not limited to, mitigating or avoiding toxicities, adverse events or adverse symptoms, or combinations thereof, associated with the compound of Formula (I), or pharmaceutically acceptable form thereof, or associated with osimertinib, or pharmaceutically acceptable form thereof. In some embodiments, the pharmaceutically acceptable salt of osimertinib is osimertinib mesylate.

In some embodiments, the methods provided herein comprise administering the compound of Formula (I) or pharmaceutically acceptable form thereof concurrently or sequentially with the EGFR-TKI such as osimertinib or pharmaceutically acceptable salt thereof. In some embodiments, the compound of Formula (I) or pharmaceutically acceptable form thereof is administered before, during or after the administration of the EGFR-TKI such as osimertinib or pharmaceutically acceptable salt thereof. In some embodiments, the dose of the compound of Formula (I), or pharmaceutically acceptable form thereof, and the dose of the osimertinib, or pharmaceutically acceptable salt thereof, are administered to the subject concurrently or sequentially. In some embodiments, the dose of the compound of Formula (I), or pharmaceutically acceptable form thereof, is administered to the subject before the administration of the dose of the osimertinib, or a pharmaceutically acceptable salt thereof. In some embodiments, the dose of the compound of Formula (I), or pharmaceutically acceptable form thereof, is administered to the subject after the administration of the dose of the osimertinib, or pharmaceutically acceptable salt thereof. In some embodiments, the dose of the compound of Formula (I), or pharmaceutically acceptable form thereof, is administered to the subject QD or BID on days 1-7, days 1-7 and 15-21, days 1-21, or each day, of a 28-day treatment cycle, and the dose of the osimertinib, or pharmaceutically acceptable salt thereof, is administered QD or BID each day of the 28-day treatment cycle. For example, in some embodiments, the dose of the compound of Formula (I), or pharmaceutically acceptable form thereof, is administered to the subject QD on days 1-7, days 1-7 and 15-21, days 1-21, or each day, of a 28-day treatment cycle, and the dose of the osimertinib, or pharmaceutically acceptable salt thereof, is administered QD each day of the 28-day treatment cycle. In some embodiments, the dose of the compound of Formula (I), or pharmaceutically acceptable form thereof, is administered to the subject BID on days 1-7, days 1-7 and 15-21, days 1-21, or each day, of a 28-day treatment cycle, and the dose of the osimertinib, or pharmaceutically acceptable salt thereof, is administered QD each day of the 28-day treatment cycle. In some embodiments, the dose of the compound of Formula (I), or pharmaceutically acceptable form thereof, is administered to the subject QD on days 1-7, days 1-7 and 15-21, days 1-21, or each day, of a 28-day treatment cycle, and the dose of the osimertinib, or pharmaceutically acceptable salt thereof, is administered BID each day of the 28-day treatment cycle. In some embodiments, the dose of the compound of Formula (I), or pharmaceutically acceptable form thereof, is administered to the subject BID on days 1-7, days 1-7 and 15-21, days 1-21, or each day, of a 28-day treatment cycle, and the dose of the osimertinib, or pharmaceutically acceptable salt thereof, is administered BID each day of the 28-day treatment cycle. In some embodiments, the pharmaceutically acceptable salt of osimertinib is administered. In some embodiments, the pharmaceutically acceptable salt of osimertinib is osimertinib mesylate.

In some embodiments, the methods comprise administering the compound of Formula (I), or pharmaceutically acceptable salt thereof, on a delayed dosing schedule and/or an interval dosing schedule. In some embodiments, the methods provided herein comprise administering the compound of Formula (I), or pharmaceutically acceptable salt thereof, or the pharmaceutical composition comprising the same, to the patient once or twice per day on days 1-7, on days 1-7 and 15-21, on days 1-21, or each day of a 28-day treatment cycle, and administering the EGFR-TKI such as osimertinib, or pharmaceutically acceptable salt thereof, or the pharmaceutical composition comprising the same, to the patient once or twice per day of the 28-day treatment cycle.

In some embodiments, the methods provided herein provide a synergistic benefit, such as a therapeutic benefit to the subject. In some embodiments, the methods provided herein improve efficacy (e.g., suppresses tumor growth and induces tumor regression). In some embodiments, the methods provided herein provide unexpected synergistic efficacy, wherein the methods increase PFS and/or OS. In some embodiments, the increased PFS is by 10-99%, such as by 10%, 25%, 50%, 80%, 90% 95%, or 99% 2 fold, 3 fold, or 4 fold. In some embodiments, the increased OS is by 10-99%, such as by 10%, 25%, 50%, 80%, 90% 95%, or 99% 2 fold, 3 fold, or 4 fold. In some embodiments, the methods provided herein reduce the therapeutically effective amount of the osimertinib, or pharmaceutically acceptable salt thereof, such as osimertinib mesylate, relative to the therapeutically effective amount required for osimertinib mesylate monotherapy. In some embodiment, the therapeutically effective amount of the osimertinib mesylate, is reduced relative to the therapeutically effective amount required for osimertinib mesylate monotherapy, for example is reduced from 80 mg to 60 mg once daily, or reduced from 80 mg to 40 mg once per day, or reduced from 60 mg to 40 mg once per day. In some embodiments, the methods provided herein reduce osimertinib-associated toxicity. In some embodiments, the reduced toxicity comprises or consists of reduced leukopenia, lymphopenia, thrombocytopenia, diarrhea, anemia, rash, musculoskeletal pain, nail toxicity, neutropenia, dry skin, stomatitis, fatigue, cough, or a combination thereof. In some embodiments, the methods provided herein delay emergence of osimertinib resistance. In some embodiments, the methods provided herein unexpectedly delay emergence of osimertinib resistance. In some embodiments, the delay in emergence of osimertinib resistance comprises weeks, months, or years. In some embodiments, according to the methods provided herein, the compound of Formula (I), or pharmaceutically acceptable form thereof, and the osimertinib, or pharmaceutically acceptable salt thereof, unexpectedly act synergistically. In some embodiments, the pharmaceutically acceptable salt of osimertinib is administered according to the methods provided herein. In some embodiments, the pharmaceutically acceptable salt of osimertinib is osimertinib mesylate.

### 7. EXAMPLES

### Example 1: Preparation of Compounds of Formula (I), (II), and (III)

Abbreviations: ACN: Acetonitrile; AIBN: Azobisisobutyronitrile; BTEAC: Benzyltriethylammonium chloride; Cu (OAc) 2: Cupric acetate; DCE: 1,2-Dichloroethane; DCM: Dichloromethane; DEA: Diethylamine; DEAD: Diethyl azodicarboxylate; DIAD: Diisopropyl azodicarboxylate; DIBAL-H: Diisobutylaluminium hydride; DIPEA: N,N-Diisopropylethylamine; DIPEA: N,N-Diisopropylethylamine; DMA: Dimethylacetamide; DMF: Dimethylformamide; DMI: 1,3-Dimethyl-2-imidazolidinone; DMSO: Dimethyl sulfoxide; DPPF: 1,1′-Bis(diphenylphosphino) ferrocene; Et₃SiCl: Chlorotriethylsilane; EtOAc: Ethyl acetate; EtOH: Ethanol; HATU: 1-[Bis (dimethylamino)methylene]-1H-1,2,3-triazolo[4,5-b]pyridinium 3-oxide hexafluorophosphate; MeOH: Methanol; NaOMe: Sodium methoxide; NBS: N-Bromosuccinimide; n-BuLi: n-Butyllithium; PCC: Pyridinium chlorochromate; Pd(Ph₃)₄: Tetrakis (triphenylphosphine) palladium (0); Pd₂ (dba)₃: Tris(dibenzylideneacetone) dipalladium (0); PPh₃: Triphenylphosphine; SFC: Supercritical fluid chromatography; T₃P: Propanephosphonic acid anhydride; TBAF: Tetra-n-butylammonium fluoride; t-BuOK: Potassium tert-butoxide; TEA: Triethylamine; TFA: Trifluoroacetic acid; THF: Tetrahydrofuran; TIPSCl: Triisopropylsilyl chloride; TMEDA: Tetramethylethylenediamine

Each LCMS conditions were conducted on instrument SHIMADZU LC20-MS2020, at an oven temperature of 50° C., with an ESI mass spectrometry ionization, monitored at wavelengths 220 nm and 254 nm. It is understood that the molecular formula listed with the ESI calculated is the molecular formula of the detected ion (e.g., [M+H]⁺). For example, the molecular formula of compound 1A-1 is C₁₇H₁₂BrNO (i.e., [M]), while the molecular formula listed with the ESI calculated is the molecular formula of the detected ion, C₁₇H₁₃BrNO (i.e., [M+H]⁺).

The acidic LCMS methods are referred to with “AB” notation. Each of the acidic LCMS methods utilized a Xtimate C18 2.1×30 mm (3 μm particle size) column (except where indicated), mobile phase A (water (4 L) and TFA (1.5 mL)), and mobile phase B (ACN (4 L) and TFA (0.75 mL)) (except where indicated). The conditions for each of the acidic LCMS methods utilized includes the following: 1.5 min method 5-95AB refers to using MERCK, RP-18e, 25×2 mm column, with a gradient starting at 5% B and ending at 95% B, over a total time of 1.5 min, and at a flow rate of 1.5 mL/min.

The basic LCMS methods are referred to with “CD” notation. Each of the basic LCMS methods utilized a Titank C18 2.1×50 mm (5 μm particle size) column, mobile phase A (water (4 L) and ammonium hydroxide (0.8 mL)), and mobile phase B (ACN). The conditions for each of the basic LCMS methods utilized includes the following: 3.0 min method 10-80CD refers to a gradient starting at 10% B and ending at 80% B, over a total time of 3 min, and at a flow rate of 1.0 mL/min.

### SFC Chiral HPLC Conditions:

Each SFC Chiral HPLC methods was conducted on either (1) Waters UPCC with PDA detector and QDa detector or (2) Agilent 1260 with DAD detector.

“AD_ETOH_DEA_5_40_4ML_4_MIN_5 CM” refers to using a Chiralpak AD-3 chiral column (5 cm column length), with CO₂ (mobile phase A) and ethanol having 0.05% of diethylamine (v/v) (mobile phase B), and using a 5% B to 40% B gradient over a total time of 4 min. at a flow rate of 4 mL/min.

The following Examples are presented by way of illustration, not limitation.

It is understood that reference to a compound as disclosed herein having one or more stereocenters without designating the specific chirality (e.g., R- or S-enantiomer) will be understood to refer to the compound as racemic mixture (or a mixture of diastereomers), while inclusion of R- or S-designations will be understood to refer to an enantiomer (or a diastereomer) form of the compound, such as an enantiomerically (or diastereomerically) enriched form of the compound, or an enantiomeric excess of the specified enantiomer form of the compound, in accordance with discussion above regarding enantiomeric enriched and enantiomeric excess. Notation of a compound with an R- or S-designation is understood to include an enantiomerically enriched or an enantiomeric excess of the specified enantiomer of the compound, and not limited to only 100% of the single specified enantiomer of the compound. For example, reference to Compound of Formula (III) will be understood to refer to the compound prepared in Example 1 and in its racemic form: (rac)-3-amino-3-(1-methyl-1H-imidazol-5-yl)-6-oxa-2 (4,6)-quinolina-1,4 (1,3)-dibenzenacyclohexaphane-2²,4⁴-dicarbonitrile. Similarly, reference to Compound of Formula (I) will be understood to refer to the compound prepared in Example 1 and in its single stereoisomer(S) form: (S)-3-amino-3-(1-methyl-1H-imidazol-5-yl)-6-oxa-2 (4,6)-quinolina-1,4 (1,3)-dibenzenacyclohexaphane-2²,4⁴-dicarbonitrile.

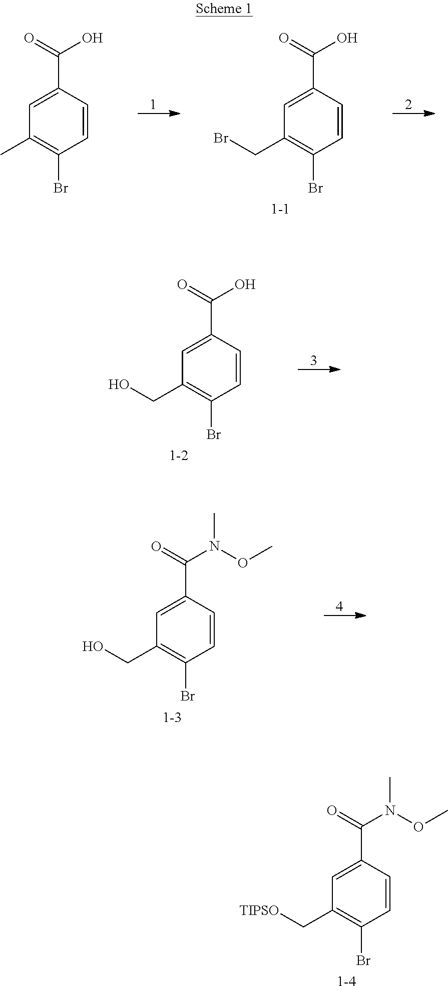

Scheme 1, Step 1: Preparation of (1-1). A mixture of 4-bromo-3-methylbenzoic acid (200 g, 930.04 mmol), NBS (248.29 g, 1.40 mol) and AIBN (30.54 g, 186.01 mmol) in CCl₄ (1600 mL) was degassed and purged with N₂ 3 times, and then the mixture was stirred at 85° C. for 12 h under N₂. The reaction mixture was filtered. The crude product was triturated by CH₃CN (500 mL) to give 1-1 and the corresponding dibromomethyl compound (215 g, 731.44 mmol, 78.65% yield) as a yellow solid. ¹H NMR (400 MHz, DMSO-d₆) δ=13.36 (br s, 1H), 8.17 (s, 1H) 7.78-7.82 (m, 2H), 4.82 (s, 2H).

Scheme 1, Step 2: Preparation of (1-2). To a solution of 1-1 and the corresponding dibromomethyl compound (160 g, 544.33 mmol) in H₂O (1500 mL) was added Na₂CO₃ (230.77 g, 2.18 mol). The mixture was stirred at 75° C. for 12 h. The reaction mixture was adjusted by HCl (4 M in H₂O) to give a white cake. The solvent was removed from the white cake. To the above product in MeOH (1000 mL) was added NaBH₄ (24.00 g, 634.42 mmol) under N₂, and then the mixture was stirred at 15° C. for 1 h under N₂. The reaction mixture was quenched by H₂O (400 mL) and acidized by HCl (1 M in H₂O) to pH=2. The mixture was placed under reduced pressure to remove the solvent and then filtered. The white filter cake was placed under the reduced pressure to remove the surplus solvent to give 1-2 (120 g, 519.38 mmol, 95.42% yield) as a yellow solid. 1H NMR (400 MHz, DMSO-d₆) δ=8.08-8.15 (m, 1H), 7.66-7.76 (m, 2H), 4.53 (s, 2H).

Scheme 1, Step 3: Preparation of (1-3). To a solution of 1-2 (100 g, 432.90 mmol), N,O-dimethylhydroxylamine (57.97 g, 594.31 mmol, HCl) and DIPEA (223.76 g, 1.73 mol, 301.56 mL) in DCM (1000 mL) was added T3P (275.43 g, 865.64 mmol, 257.41 mL). The mixture was stirred at 15° C. for 5 min. Water (200 mL) was added to the reaction mixture and then extracted with DCM (500 mL×2). The organic layers was separated, and washed with brine (200 mL), dried over anhydrous Na₂SO₄, filtered and concentrated. The residue was purified by flash chromatography on silica gel (EtOAc in petroleum ether=0% to 35%) to give 1-3 (83 g, 302.80 mmol, 69.95% yield) as a colorless oil. 1H NMR (400 MHz, DMSO-d₆) δ=7.92 (s, 1H), 7.72-7.76 (m, 1H), 7.64 (d, J=8.4 Hz, 1H), 7.41 (dd, J=8.4, 2.0 Hz, 1H), 4.53 (d, J=5.6 Hz, 2H), 3.54 (s, 3H), 3.26 (s, 3H).

Scheme 1, Step 4: Preparation of 4-bromo-N-methoxy-N-methyl-3-(((triisopropylsilyl)oxy)methyl)benzamide (1-4). A solution of 1-3 (83 g, 302.80 mmol), TIPSCl (58.5 g, 303.42 mmol, 64.93 mL) and imidazole (51.54 g, 756.99 mmol) in DCM (800 mL) was stirred at 15° C. for 16 h. The reaction mixture was diluted with H₂O (500 mL) and extracted with DCM (600 mL×2). The combined organic layers were washed with brine (400 mL), dried over Na₂SO₄, filtered and concentrated under reduced pressure to give a residue. The crude was purified by flash chromatography on silica gel (EtOAc in petroleum ether=0 to 10%) to give 1-4 (104 g, 241.60 mmol, 79.79% yield) as a colorless oil. 1H NMR (400 MHz, DMSO-d₆) δ=7.81 (d, J=2.0 Hz, 1H), 7.67 (d, J=8.4 Hz, 1H), 7.46 (dd, J=8.4, 2.4 Hz, 1H), 4.80 (s, 2H), 3.51 (s, 3H), 3.23-3.29 (m, 3H), 1.12-1.23 (m, 3H), 1.03-1.08 (m, 18H).

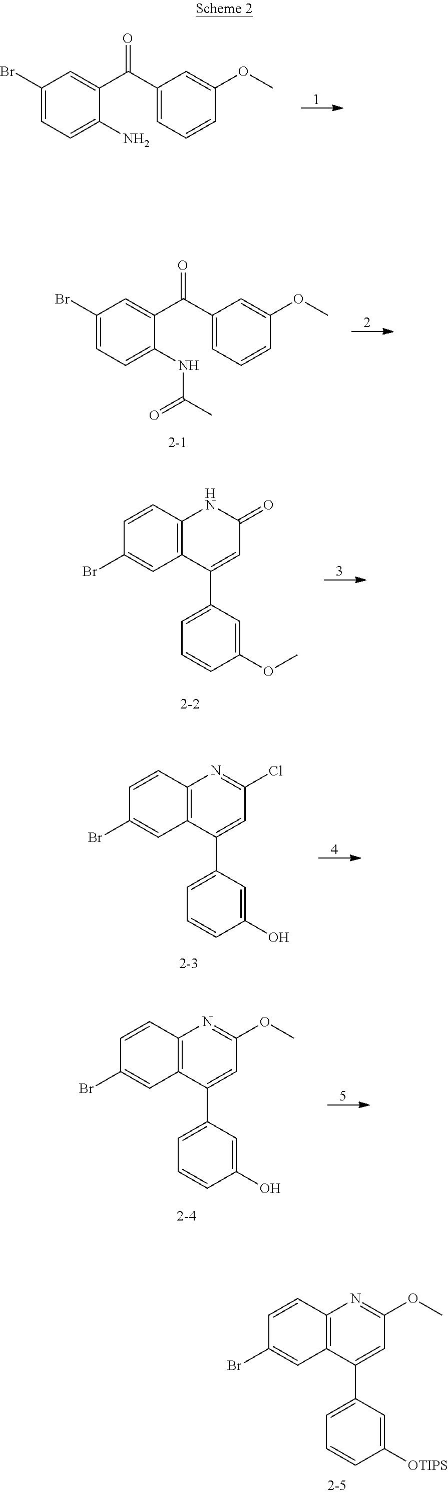

Scheme 2, Step 1: Preparation of 2-1. To a mixture of (2-amino-5-bromophenyl) (3-methoxyphenyl) methanone (500 g, 1.63 mol) in toluene (3000 mL) was added Ac₂O (333.46 g, 3.27 mol, 305.93 mL) and the mixture was stirred at 110° C. for 14 h. The reaction mixture was concentrated under reduced pressure to obtain 2-1 (528 g, 1.52 mol, 92.85% yield) as a brown solid. LC-MS: Method: 5-95AB, Rₜ=0.88 min, M/Z calculated for C₁₆H₁₅BrNO₃ [M+H]⁺350.0, found 349.9.

Scheme 2, Step 2: Preparation of 2-2. To a solution of 2-1 (528 g, 1.52 mol) in DME (2000 mL) under ice water was added t-BuOK (340.31 g, 3.03 mol) in portions, while maintaining the temperature at 20° C. under N₂. The resulting mixture was stirred at 20° C. for 12 h after which the reaction was quenched by water (200 mL). The mixture was concentrated under reduced pressure to remove DME. The residue was triturated with water (2000 mL, twice) then stirred with EtOAc (1000 mL) at 25° C. for 1 h to give 2-2 (487 g, 1.47 mol, 97.27% yield) as yellow solid. 1H NMR (400 MHz, DMSO-d₆) δ=7.73-7.64 (m, 1H), 7.50-7.34 (m, 3H), 7.15-6.93 (m, 3H), 6.46 (s, 1H), 3.81 (s, 3H).

Scheme 2, Step 3: Preparation of 2-3. To a solution of 2-2 (50 g, 143.42 mmol) in DCM (500 mL) at −40° C. under N₂ was added BBr₃ (53.90 g, 215.13 mmol, 20.73 mL). The mixture was stirred at 25° C. for 4 h. The reaction mixture was poured into water (500 mL). The pH was adjusted to 7 with saturated NaHCO₃ solution. The aqueous layer was extracted with DCM (300 mL×2). The combined organic phase was washed with brine (300 mL), dried over anhydrous Na₂SO₄, filtered, and concentrated. The crude product was triturated with petroleum ether (300 mL) at 25° C. for 30 min and CH₃CN (200 mL) at 25° C. for 30 min to give 2-3 (42 g, 125.53 mmol, 87.52% yield) as a yellow solid. 1H NMR (400 MHz, DMSO-d₆) δ=9.92 (br s, 1H), 8.03-7.91 (m, 3H), 7.56 (s, 1H), 7.43-7.37 (m, 1H), 6.99-6.91 (m, 3H).

Scheme 2, Step 4: Preparation of 2-4. To a solution of 2-3 (170 g, 508.08 mmol) in MeOH (800 mL) and THF (800 mL) at 25° C. was added CH₃ONa (54.89 g, 1.02 mol), and the mixture was stirred at 80° C. for 12 h. The solvents were removed under reduced pressure. The mixture was poured into water (1000 mL), stirred for 30 min, then filtered. The filtrate was concentrated under reduced pressure. The crude product was triturated with CH₃CN (500 mL) at 25° C. for 30 min to give 2-4 (130 g, 393.73 mmol, 67.98% yield) as a yellow solid. 1H NMR (400 MHz, DMSO-d₆) δ=7.79 (s, 3H), 7.43-7.29 (m, 1H), 6.99-6.84 (m, 4H), 4.01 (s, 3H), 3.64 (s, 1H).

Scheme 2, Step 4: Preparation of 6-bromo-2-methoxy-4-(3-((triisopropylsilyl)oxy)phenyl)-quinoline (2-5). Imidazole (58.97 g, 866.21 mmol) was added to a solution of 2-4 (130 g, 393.73 mmol) in DCM (1500 mL) under N₂ at 0° C. The mixture was stirred until a clear solution appeared, TIPSCl (75.91 g, 393.73 mmol, 84.25 mL) was added dropwise, and the mixture was stirred at 0° C. for 1 h after which the ice bath was removed, and the mixture was stirred at 25° C. for 12 h. The residue was poured into water (1000 mL) and then extracted with DCM (1000 mL×3). The combined organic phase was washed with brine (1000 mL), dried over anhydrous Na₂SO₄, filtered, and concentrated under reduced pressure. The crude was purified by flash chromatography on silica gel (EtOAc in petroleum ether=0 to 5%) and then triturated with MeOH (300 mL) at 25° C. for 30 minutes to give 2-5 (160 g, 328.87 mmol, 83.52% yield) as a yellow solid. 1H NMR (400 MHz, CDCl₃) δ=7.88 (d, J=2.0 Hz, 1H), 7.79-7.73 (m, 1H), 7.67 (dd, J=2.4 Hz, J=9.2 Hz, 1H), 7.35 (t, J=8.0 Hz, 1H), 7.03-6.97 (m, 2H), 6.96-6.93 (m, 1H), 6.83 (s, 1H), 4.07 (s, 3H), 1.32-1.23 (m, 3H), 1.13-1.09 (m, 18H).

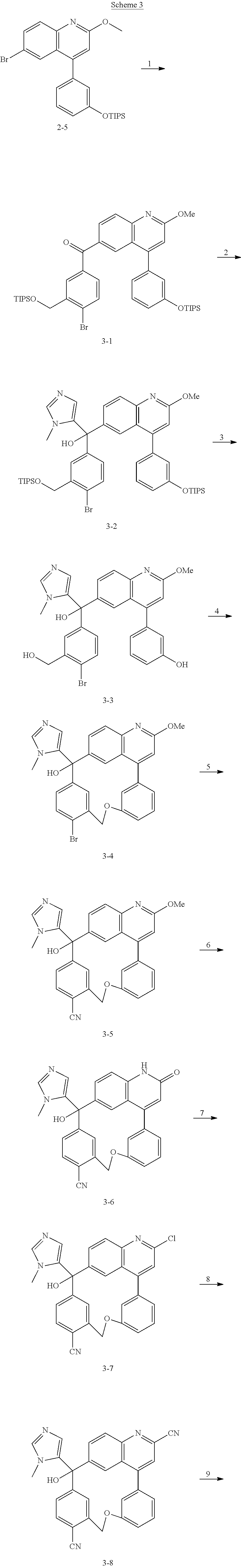

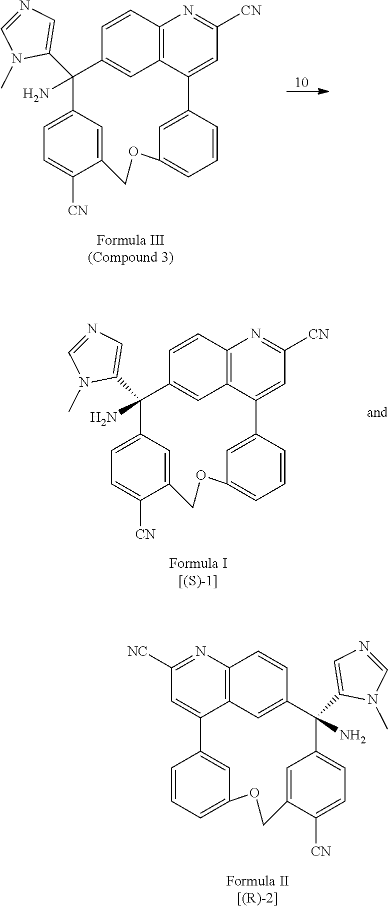 [SMILES: Cn1cncc1C1(N)c2ccc(C#N)c(c2)COc2cccc(c2)-c2cc(C#N)nc3ccc1cc23 ; "10" ; Cn1cncc1[C@@]1(N)c2ccc(C#N)c(c2)COc2cccc(c2)-c2cc(C#N)nc3ccc1cc23 ; Cn1cncc1[C@]1(N)c2ccc(C#N)c(c2)COc2cccc(c2)-c2cc(C#N)nc3ccc1cc23]

Scheme 3, Step 1: Preparation of (3-1). To a solution of 6-bromo-2-methoxy-4-(3-((triisopropylsilyl)oxy)phenyl) quinoline (10 g, 20.55 mmol) in THF (100 mL) was added n-BuLi (2.5 M in n-hexane, 22.61 mmol, 9.04 mL) and the mixture was stirred at −70° C. under N₂ for 0.5 h. A solution of 1-4 (9.00 g, 20.91 mmol) in THF (10 mL) was added to the above solution and the mixture was stirred at −70° C. for 0.5 h. Water (150 mL) was added to the mixture and the mixture was extracted with EtOAc (150 mL). The organic phase was washed with brine (150 mL), dried over anhydrous Na₂SO₄, filtered and concentrated. The mixture was blended with another batch prepared from 18 g of 6-bromo-2-methoxy-4-(3-((triisopropylsilyl)oxy)phenyl) quinoline. The crude was purified by flash chromatography on silica gel (EtOAc in petroleum ether=0 to 5%) to give 3-1 (40 g, 51.48 mmol, 83.50% yield) as yellow oil. ¹H NMR (400 MHz, CDCl₃) δ=8.28 (s, 1H), 8.09-7.95 (m, 3H), 7.62-7.54 (m, 2H), 7.35-7.29 (m, 1H), 7.09-7.04 (m, 1H), 7.00-6.95 (m, 2H), 6.92 (s, 1H), 4.85 (s, 2H), 4.16 (s, 3H), 1.28-1.21 (m, 3H), 1.16-1.13 (m, 3H), 1.11-1.07 (m, 18H), 1.04-1.01 (m, 18H).

Scheme 3, Step 2: Preparation of (3-2). To a solution of 1-methyl-1H-imidazole (1.16 g, 14.16 mmol, 1.13 mL) in THF (50 mL) was added n-BuLi (2.5 M in n-hexane, 14.16 mmol, 5.66 mL) and the mixture was stirred at −70° C. under N₂ for 20 min. Then Et₃SiCl (2.13 g, 14.16 mmol, 2.41 mL) in THF (10 mL) was added to the above mixture and the mixture was stirred at −70° C. for 20 min. Then n-BuLi (2.5 M in n-hexane, 14.16 mmol, 5.66 mL) was added to the above mixture and the mixture was stirred at −70° C. for 20 min. Then 3-1 (10 g, 12.87 mmol) in THF (40 mL) was added to the above mixture and the mixture was stirred at −70° C. for 20 min. Water (500 mL) was added to the mixture and the mixture was extracted with EtOAc (500 mL). The organic phase was washed with brine (250 mL), dried over anhydrous Na₂SO₄, filtered and concentrated. The mixture was blended with another batch prepared from 30 g of 3-1. The crude was purified by flash chromatography on silica gel (MeOH in DCM=0 to 10%) to give 3-2 (34 g, 39.58 mmol, 76.88% yield) as a light yellow solid. 1H NMR (400 MHz, CDCl₃) δ=7.70-7.63 (m, 2H), 7.36-7.32 (m, 1H), 7.30-7.25 (m, 2H), 7.11-7.04 (m, 3H), 6.81-6.76 (m, 2H), 6.75-6.71 (m, 1H), 6.69 (s, 1H), 6.14 (s, 1H), 4.63-4.54 (m, 2H), 3.96 (s, 3H), 3.18 (s, 3H), 1.13-1.06 (m, 3H), 0.96-0.92 (m, 18H), 0.89-0.84 (m, 3H), 0.80-0.77 (m, 18H).

Scheme 3, Step 3: Preparation of (3-3). A mixture of 3-2 (26.5 g, 30.85 mmol) and TBAF (1 M in THF, 46.27 mmol, 46.27 mL) in THF (250 mL) was stirred at 25° C. for 20 min. Water (500 mL) was added to the mixture and the mixture was extracted with EtOAc (500 mL). The organic phase was washed with brine (500 mL), dried over anhydrous Na₂SO₄, filtered and concentrated. The crude was purified by triturated from EtOAc: petroleum ether=1:5 (150 mL) to give 3-3 (16 g, 29.28 mmol, 94.93% yield) as an off-white solid. 1H NMR (400 MHz, DMSO-d₆) δ=9.69 (s, 1H), 7.81 (d, J=8.8 Hz, 1H), 7.74-7.68 (m, 1H), 7.64-7.54 (m, 2H), 7.53-7.44 (m, 2H), 7.29-7.21 (m, 1H), 7.04-6.96 (m, 1H), 6.91-6.84 (m, 3H), 6.83-6.75 (m, 2H), 6.05 (s, 1H), 5.42-5.35 (m, 1H), 4.52-4.41 (m, 2H), 4.03-3.98 (m, 3H), 3.35 (s, 3H). LCMS Rₜ=0.80 min in 1.5 min chromatography, 5-95AB, ESI calcd, for C₂₈H₂₅BrN₃O₄ [M+H]⁺546.1, found 545.9.

Scheme 3, Step 4: Preparation of (3-4). To a solution of 3-3 (5.7 g, 10.43 mmol) in DMF (85 mL) was added SOCI2 (2.48 g, 20.86 mmol, 1.51 mL) and the mixture was stirred at 25° C. for 1 h. To the above solution was added Cs₂CO₃ (50.96 g, 156.41 mmol) and the mixture was stirred at 70° C. for 0.5 h. After cooling to room temperature, the mixture was filtered and the filtrate was concentrated. The crude product was blended with another batch prepared from 10.15 g of 3-3 was triturated from water (150 mL) and filtered. The solid was re-dissolved in toluene (100 mL×2) and concentrated to give 3-4 (13 g, 24.60 mmol, 84.84% yield) as a light yellow solid. 1H NMR (400 MHz, DMSO-d₆) δ=8.09-8.01 (m, 1H), 7.92-7.87 (m, 1H), 7.69-7.53 (m, 4H), 7.38-7.34 (m, 1H), 7.26 (s, 1H), 7.19-7.10 (m, 3H), 7.07-7.00 (m, 2H), 6.29 (s, 1H), 5.49-5.37 (m, 2H), 4.05 (s, 3H), 3.50 (s, 3H). LCMS Rₜ=2.00 min in 3.0 min chromatography, 10-80CD, ESI calcd, for C₂₈H₂₃BrN₃O₃ [M+H]⁺530.1, found 530.1.

Scheme 3, Step 5: Preparation of (3-5). A mixture of 3-4 (12 g, 22.71 mmol), Zn (CN) 2 (27.23 g, 231.89 mmol, 14.72 mL), Pd₂ (dba)₃ (3.12 g, 3.41 mmol), dppf (3.78 g, 6.81 mmol) and Zn (891.01 mg, 13.63 mmol) in DMA (300 mL) was stirred at 120° C. under N₂ for 2 h. The mixture was filtered through Celite. The cake was washed with EtOAc (100 mL×2). The combined organic phase was concentrated. The crude was purified by flash chromatography on silica gel (EtO Ac in petroleum ether=50% to 100%) and then triturated with MeOH (50 mL) to give 3-5 (6.55 g, 13.80 mmol, 60.78% yield) as a yellow solid. 1H NMR (400 MHz, DMSO-d₆) δ=8.05-8.01 (m, 1H), 7.95-7.90 (m, 1H), 7.85-7.78 (m, 2H), 7.63-7.44 (m, 2H), 7.40-7.32 (m, 2H), 7.18 (s, 1H), 7.12 (d, J=7.6 Hz, 1H), 7.07-7.01 (m, 3H), 6.94 (s, 1H), 5.52 (s, 2H), 4.06 (s, 3H), 3.62 (s, 3H). LCMS Rₜ=1.77 min in 3.0 min chromatography, 10-80CD, ESI calcd, for C₂₉H₂₃N₄O₃ [M+H]′ 475.2, found 475.2.

Scheme 3, Step 6: Preparation of (3-6). To a solution of 3-5 (0.12 g, 251.82 μmol) in THF (10 mL) was added HCl (4 M in H₂O, 2.20 mL). The reaction mixture was stirred at 70° C. for 16 h. The mixture was cooled to 20° C. and added into water (20 mL). Saturated NaHCO₃ solution was added to adjust pH=8. The aqueous phase was extracted with DCM (30 mL×2). The combined organic phase was washed with brine (50 mL), dried over anhydrous Na₂SO₄, filtered and concentrated under reduced pressure to give 3-6 (0.115 g, 249.74 μmol, 99.17% yield) as a colorless oil. 50 mg (108.58 μmol) of 3-6 was purified by Prep-HPLC (column: Phenomenex Gemini-NX 80×40 mm×3 μm; mobile phase: [water (10 mM NH₄HCO₃)-ACN]; B %: 26%-56%, 7.8 min) to give 3-6 (12.4 mg, 26.93 μmol, 24.80% yield) as an off-white solid. 1H NMR (400 MHz, DMSO-d₆) δ=11.71 (br s, 1H), 7.89 (dd, J=2.0, 8.4 Hz, 1H), 7.81 (d, J=7.8 Hz, 1H), 7.69 (s, 1H), 7.57 (s, 1H), 7.45 (d, J=8.8 Hz, 2H), 7.38-7.31 (m, 1H), 7.10-7.02 (m, 3H), 6.75 (s, 1H), 6.65 (s, 1H), 6.52 (s, 1H), 6.34 (s, 1H), 5.55-5.46 (m, 2H), 3.49 (s, 3H). LCMS Rₜ=1.34 min in 3 min chromatography, 10-80CD, ESI calcd. for: C₂₈H₂₁N₄O₃ [M+H]⁺461.2, found 461.1. HPLC Rₜ=2.22 min in 8 min chromatography, 220 nm, purity 100%.

Scheme 3, Step 7: Preparation of (3-7). Compound 3-6 (1.2 g, 2.61 mmol) was mixed with POCl₃ (19.80 g, 129.13 mmol, 12.00 mL) at 25° C. The mixture was stirred at 100° C. for 1 h. The mixture was concentrated. To the residue was added NaOH (1 M in H₂O, 100 mL). The aqueous layer was extracted with EtOAc (200 mL×2). The combined organic layers were washed with brine (50 mL×2), dried over anhydrous Na₂SO₄, filtered and the filter cake was washed with EtOAc (20 mL). The combined filtrates were concentrated. The crude product was blended with another batch prepared from 0.5 g of 3-6. The crude product was purified by flash chromatography on silica gel (MeOH in DCM=0 to 10%) to give 3-7 (1.3 g, 2.71 mmol, 73.35% yield) as a yellow solid. LCMS Rₜ=1.79 min in 3.0 min chromatography, 10-80 CD, ESI calcd, for C₂₈H₂₀ClN₄O₂ [M+H]⁺479.1, found 479.1.

Scheme 3, Step 8: Preparation of (3-8). To a solution of 3-7 (1.2 g, 2.51 mmol) in DMF (10 mL) was added Zn (CN) 2 (2.69 g, 22.91 mmol, 1.45 mL) and Pd(PPh₃)₄ (579.07 mg, 501.12 μmol) in a three-neck bottom flask at 25° C. under N₂. The mixture was stirred at 100° C. for 2 h. The mixture was cooled to 25° C. and added into water (50 mL). The aqueous phase was extracted with EtOAc (50 mL×2). The combined organic phase was washed with brine (50 mL×2), dried over anhydrous Na₂SO₄, filtered and concentrated. The crude product was purified by flash chromatography on silica gel (MeOH in DCM=0 to 3%) to give 3-8 (900 mg, 1.92 mmol, 76.51% yield) as a yellow solid. 1H NMR (400 MHz, DMSO-d₆) δ=8.33-8.22 (m, 2H), 8.10 (s, 1H), 7.94-7.76 (m, 2H), 7.69 (s, 1H), 7.52-7.39 (m, 2H), 7.28-7.02 (m, 5H), 6.36 (s, 1H), 5.54 (s, 2H), 3.56 (s, 3H).

Scheme 3, Step 9: Preparation of (rac)-3-amino-3-(1-methyl-1H-imidazol-5-yl)-6-oxa-2 (4,6)-quinolina-1,4 (1,3)-dibenzenacyclohexaphane-2²,4⁴-dicarbonitrile (Compound of Formula III). To a solution of 3-8 (800 mg, 1.70 mmol) in DMI (8 mL) was added SOCl₂ (1.01 g, 8.52 mmol, 618.05 μL). The mixture was stirred at 40° C. for 1 h. To NH₃ in MeOH (7 M, 100 mL) was added the above mixture at −10° C. The mixture was stirred at 25° C. for 30 min. The reaction mixture was poured into H₂O (100 mL). The aqueous layer was extracted with EtOAc (150 mL×2). The combined organic layers were washed with brine (50 mL×2), dried over anhydrous Na₂SO₄, filtered and the filter cake was washed with EtOAc (20 mL). The combined filtrates were concentrated. The crude product was purified by flash chromatography on silica gel (MeOH in DCM=0 to 8%) to give Compound 3 (550 mg, 1.17 mmol, 68.89% yield) as a yellow solid. LCMS Rₜ=1.71 min in 3.0 min chromatography, 10-80CD, ESI calcd. for C₂₉H₂₁N₆O [M+H]⁺469.2, found 469.2.

Scheme 2, Step 10: Preparation of(S)-3-amino-3-(1-methyl-1H-imidazol-5-yl)-6-oxa-2 (4,6)-quinolina-1,4 (1,3)-dibenzenacyclohexaphane-2²,4⁴-dicarbonitrile (Compound of Formula (I)) and (R)-3-amino-3-(1-methyl-1H-imidazol-5-yl)-6-oxa-2 (4,6)-quinolina-1,4 (1,3)-dibenzenacyclohexaphane-2²,4⁴-dicarbonitrile (Compound of Formula (II)).

Compound of Formula (III) (500 mg, 1.07 mmol) was separated by SFC (column: DAICEL CHIRALPAK AD (250 mm×30 mm, 10 μm); mobile phase: [0.1% NH₃ in H₂O/EtOH]; B %: 45%-45%) to give the Compound of Formula (I) ((S)-1; 229.5 mg, 489.85 μmol, 45.90% yield) as an off-white solid ad Compound of Formula (II). Both enantiomers: 1H NMR (400 MHz, DMSO-d₆) δ=8.37 (d, J=8.4 Hz, 1H), 8.23 (d, J=9.2 Hz, 1H), 8.08 (s, 1H), 7.95 (s, 1H), 7.83 (d, J=8.0 Hz, 1H), 7.58 (s, 1H), 7.48-7.19 (m, 4H), 7.18-7.04 (m, 2H), 6.44 (s, 1H), 5.64-5.45 (m, 2H), 3.48 (s, 3H), 3.18 (s, 2H). Both enantiomers: LCMS Rₜ=1.68 min in 3.0 min chromatography, 10-80CD, ESI calcd, for C₂₉H₂₁N₆O [M+H]⁺469.2, found 469.2. Formula (I): HPLC Rₜ=3.03 min in 8 min chromatography, 220 nm, purity 100%. Chiral HPLC: Formula (I) ((S)-1): Rₜ=2.44 min in 4 min (ee 99.54%) (AD_ETOH_DEA_5_40_4ML_4MIN_5CM); Formula (II): Rₜ=1.93 min (ee 99.44%).

### Example 2: Combination of an FTI and an EGFR-TKI in EGFR-Mutant NSCLC Xenograft Models

Experiment A. Human cancer xenograft model LU0387 (Crown Bioscience, Beijing; adenocarcinoma; EGFR exon20-insertion at 2319 (H773-V774insNPH)) in female nude mice was used for this study. Fresh tumor tissues from mice bearing established primary human cancer tissues were harvested and cut into small pieces (approx. 2-3 mm in diameter). Each mouse was inoculated subcutaneously in the right front flank with a specific PDX tumor fragment (3×3×3 mm) for tumor development. The randomization into various treatment groups, with 8 mice per group, started when the mean tumor size reached approximately 300 mm3. Randomization was performed based on “Matched distribution” method (Study Director™ software, version 3.1.399.19). The date of grouping was denoted as day 0. After tumor inoculation, the animals were checked daily for morbidity and mortality. During routine monitoring, the animals were checked for any effects of tumor growth and treatments on behavior such as mobility, food and water consumption, body weight gain/loss (body weights were measured three times/daily per week after randomization), eye/hair matting, and any other abnormalities. Mortality and observed clinical signs were recorded for individual animals in detail. Tumor volumes were measured three times per week after randomization in two dimensions using a caliper, and the volume was expressed in mm³ using the formula: V=(L x W x W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet. The body weights and tumor volumes were measured by using Study Director™ software (version 3.1.399.19).

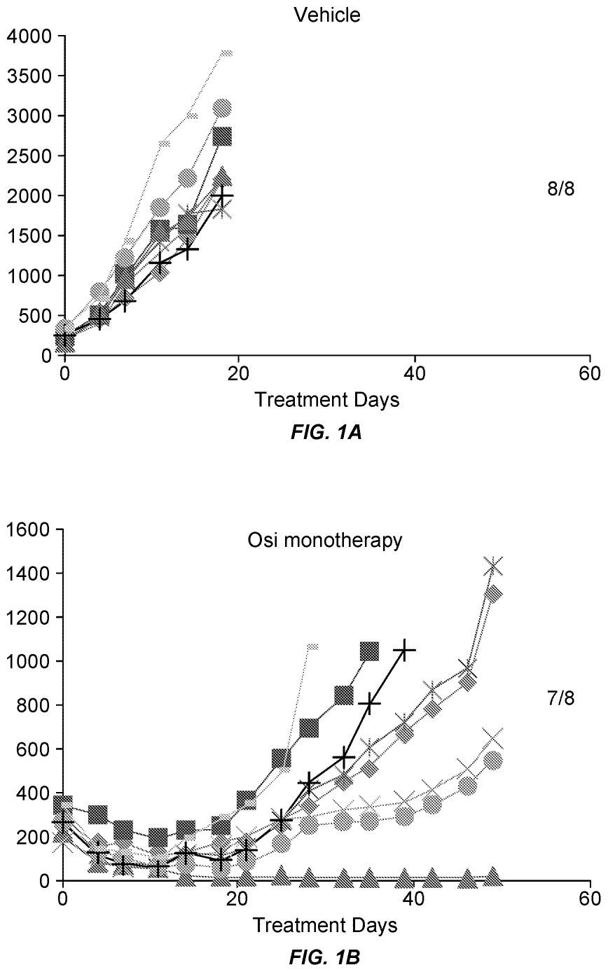

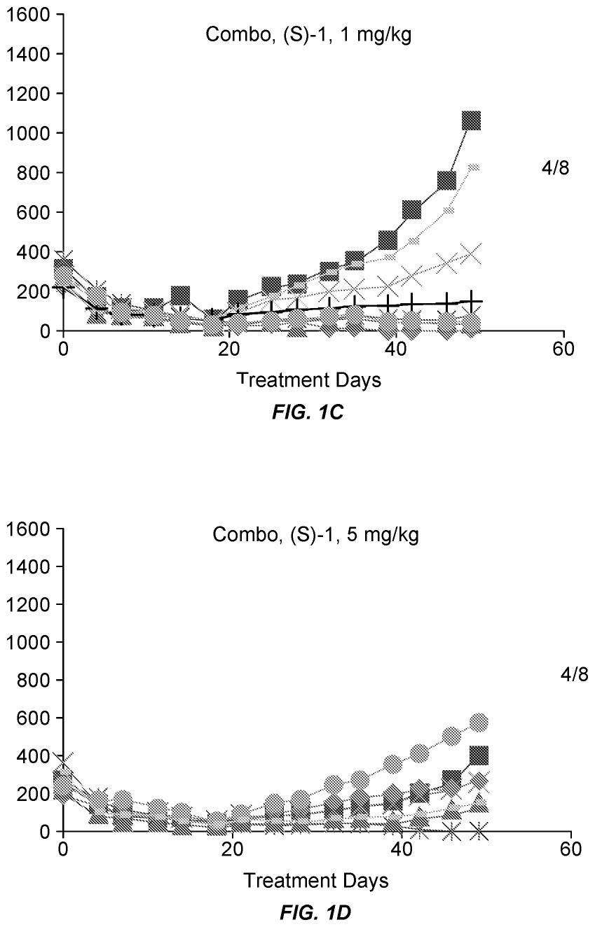

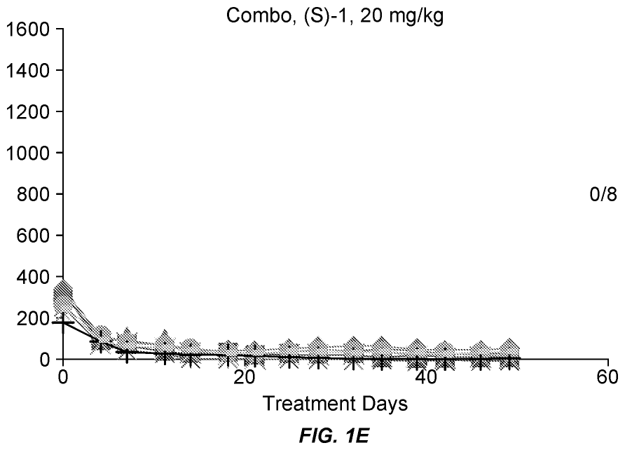

LU0387 xenografts were treated orally with: control vehicle (FIG. 1A); osimertinib, 25 mg/kg (suspension in 5% DMSO, 40% PEG300, 5% Tween 80, and 50% double distilled H₂O), QD, on a continuous dosing schedule (FIG. 1B); Compound(S)-1, 1 mg/kg, BID, plus osimertinib, 25 mg/kg, QD (FIG. 1C); Compound(S)-1, 5 mg/kg, BID, plus osimertinib, 25 mg/kg, QD (FIG. 1D); Compound(S)-1, 20 mg/kg, BID, plus osimertinib, 25 mg/kg, QD (FIG. 1E). The x/8 numbers on each figure indicate the number of animals out of 8 in each test group that were de novo resistant or that developed resistance. Xenografts treated with Compound(S)-1, BID, plus osimertinib, 25 mg/kg, QD, showed complete tumor regression, and either no or slower tumor regrowth relative to xenografts in the osimertinib single agent treatment group.

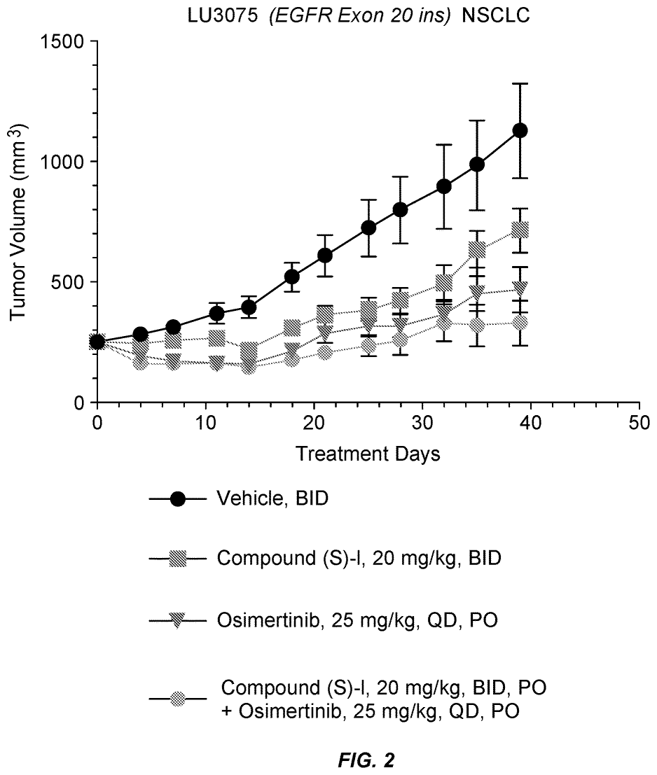

Experiment B. Using the same procedure, female nude mice were inoculated with human cancer xenograft model LU3075 (Crown Bioscience, Beijing; squamous cell carcinoma; EGFR exon 20-insertion at 2316 (P772-H773insDNP)) tumor tissues and treated orally with control vehicle; Compound(S)-1, 20 mg/kg, BID; osimertinib, 25 mg/kg, QD; Compound(S)-1, 20 mg/kg, BID, plus osimertinib, 25 mg/kg, QD. Results are shown in FIG. 2.

### Example 3: Long-Term Cell Growth Assay

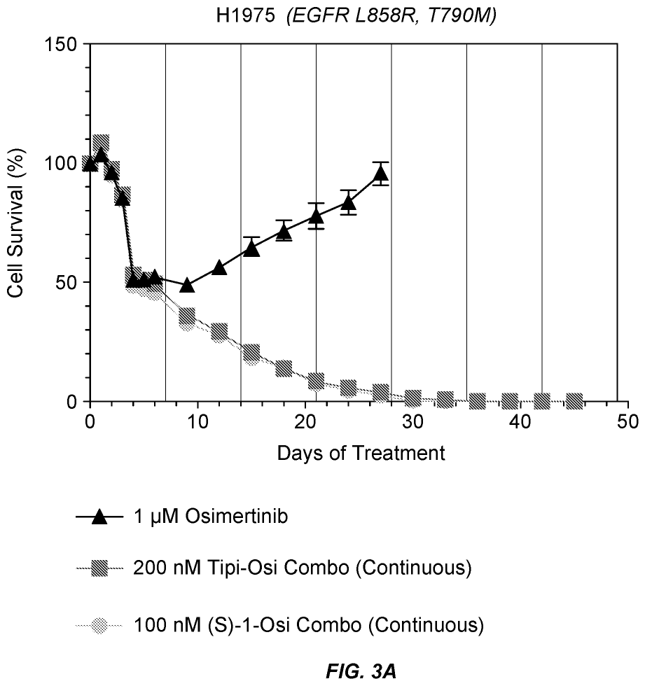

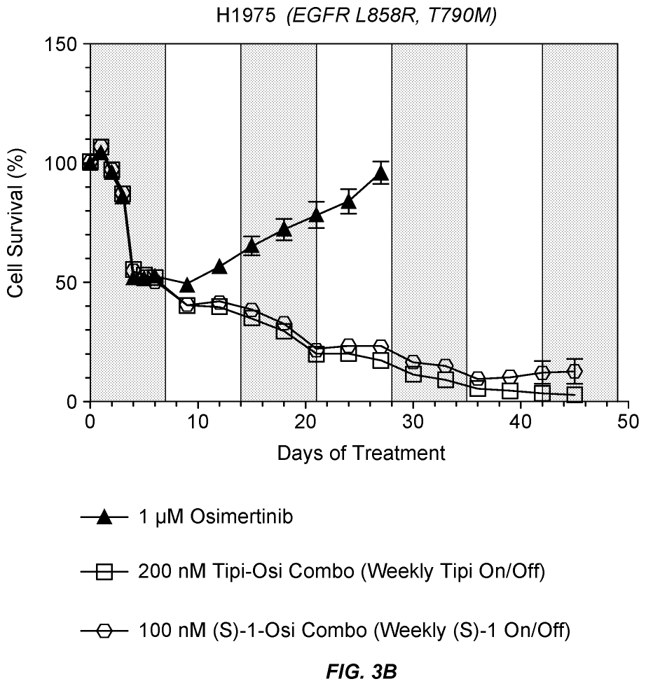

NCI-H1975 (EGFR L858R, T790M) cells were plated at a cell density of 6,000 cells/well into a 96-well plate. RPMI+10% heat-inactivated FBS medium with fresh agent was changed every 3-4 days. The cell confluency was determined daily using the Incucyte SC5 live cell analysis system (Sartorius). The cells were stably transduced with Incucyte Nuclight NIR Lentivirus (puro) for nuclear labeling to enable real-time cell counting. Treatment conditions were as follows: FIG. 3A: 1 μM osimertinib, alone or in combination with 200 nM tipifarnib or 100 nM Compound(S)-1, all on a continuous dosing schedule; FIG. 3B: 1 μM osimertinib, on a continuous dosing schedule, alone or in combination with 200 nM tipifarnib or 100 nM Compound(S)-1, each dosed on an interval dosing schedule (one week on, one week off). Continuous exposure with osimertinib and Compound(S)-1 or tipifarnib prevented cell regrowth that occurred with treatment with single agent osimertinib. Additionally, osimertinib continuous dosing in combination with interval dosing of either 100 nM Compound(S)-1 or 200 nM tipifarnib was effective, after an induction period, in preventing cell regrowth that occurred with single agent osimertinib exposure.

### Example 4: Clinical Study of the Combination of Compound(S)-1 and Osimertinib

This study is designed to evaluate the safety, tolerability and preliminary antitumor activity of the compound of Formula (I) (Compound(S)-1) in combination with osimertinib (osimertinib mesylate) in patients with treatment-naïve advanced or metastatic EGFR-mutated NSCLC. Eligible patients: (a) will have histologically or cytologically confirmed stage IIIB (locally advanced) or IV (metastatic) adenocarcinoma of the lung (e.g., this may occur as systemic recurrence after prior surgery for early-stage disease or patients may be newly diagnosed with stage IIIB/IV disease) or mixed histology if adenocarcinoma is predominant histology; (b) will have tumors confirmed to have an exon 19 deletion or a exon 21 L858R substitution, alone or in combination with other EGFR mutations (e.g., an exon 20 insertion mutation), as determined by tumor tissue or plasma (e.g., ctDNA) assessment by an FDA-approved test by a Clinical Laboratory Improvement Amendments (CLIA)-certified (US sites) or an accredited (outside the US) local laboratory that has been validated for EGFR mutation detection in NSCLC tumor tissue or plasma ctDNA; (c) will be treatment-naïve for locally advanced/metastatic EGFR-mutated NSCLC or osimertinib treatment-naïve for NSCLC; and (d) will have measurable disease by RECIST v.1.1 that meets criteria for selection as a target lesion, confirmed by radiological assessment. Additional eligibility criteria may apply.

Dosing Amounts and Regimens. Daily dosing amounts and regimens for the agents to be studied may include those listed in Table 1. Osimertinib will be administered in the form of osimertinib mesylate, and listed amounts for osimertinib are free base equivalent amounts. In the dose expansion phase, one or more dosing regimens from the dose escalation phase may be selected for continued evaluation of safety, tolerability, and preliminary efficacy in patients who are treatment-naïve for locally advanced/metastatic EGFR mutated NSCLC and osimertinib treatment-naïve for NSCLC.

TABLE 1
Daily Dosing Amounts and Regimens

| Combinations | Compound (S)-1 | | Osimertinib mesylate | |
|---|---|---|---|---|
| 1 | 1-2400 | mg QD | 10-160 | mg QD |
| 2 | 0.5-1200 | mg BID | 10-160 | mg QD |
| 3 | 1-2400 | mg QD | 10-160 | mg BID |
| 4 | 0.5-1200 | mg BID | 10-160 | mg BID |
| 5 | 1-100 | mg QD | 40 | mg QD |
| 6 | 5-50 | mg QD | 40 | mg QD |
| 7 | 100-200 | mg QD | 40 | mg QD |
| 8 | 1-100 | mg QD | 80 | mg QD |
| 9 | 5-50 | mg QD | 80 | mg QD |
| 10 | 100-200 | mg QD | 80 | mg QD |
| 11 | 0.5-100 | mg BID | 40 | mg QD |
| 12 | 5-50 | mg BID | 40 | mg QD |
| 13 | 100-200 | mg BID | 40 | mg QD |
| 14 | 0.5-100 | mg BID | 80 | mg QD |
| 15 | 5-50 | mg BID | 80 | mg QD |
| 16 | 100-200 | mg BID | 80 | mg QD |
| 17 | 1-100 | mg QD | 40 | mg BID |
| 18 | 5-50 | mg QD | 40 | mg BID |
| 19 | 100-200 | mg QD | 40 | mg BID |
| 20 | 1-100 | mg QD | 80 | mg BID |
| 21 | 5-50 | mg QD | 80 | mg BID |
| 22 | 100-200 | mg QD | 80 | mg BID |
| 23 | 0.5-100 | mg BID | 40 | mg BID |
| 24 | 5-50 | mg BID | 40 | mg BID |
| 25 | 100-200 | mg BID | 40 | mg BID |
| 26 | 0.5-100 | mg BID | 80 | mg BID |
| 27 | 5-50 | mg BID | 80 | mg BID |
| 28 | 100-200 | mg BID | 80 | mg BID |

The compound of Formula (I) or pharmaceutically acceptable form thereof will be administered on days 1 to 7 and 15 to 21 of a 28-day treatment cycle.

Compound(S)-1 will be administered on days 1 to 7 and 15 to 21 every 28 days (one cycle). Osimertinib mesylate will be administered each day for 28 days (one cycle).

Safety Evaluations. DLT will be evaluated according to NCI Common Terminology Criteria for Adverse Events (CTCAE v5.0) and will be assessed in cycle 1 (28 days) for all patients in the dose escalation phase. To be DLT evaluable, patients must receive a minimum of 10 days of Compound(S)-1 in cycle 1 at the assigned dose. Treatment-related DLTs occurring in cycle 1 are defined as: Grade ≥3 non-hematologic, non-hepatic organ system, toxicity excluding alopecia and grade 3 diarrhea, nausea, or vomiting that responds to standard-of-care therapy; Grade ≥4 thrombocytopenia; Grade ≥4 neutropenia lasting >5 days; Grade ≥3 febrile neutropenia; Grade ≥3 serum bilirubin or hepatic transaminases or values <5 times the upper limit of normal due to known liver metastases; or any clinically significant adverse event (any grade) that in the opinion of the SMC constitutes a DLT. Adverse events assessed (by the treating physician) as related to both agents and fulfilling the DLT criteria (above) will be defined as DLTs. Osimertinib mesylate may be dose reduced to 40 mg QD for treatment-related adverse events. Compound(S)-1 may be dose reduced to the next lower dose level.

Efficacy Assessments. Efficacy assessments will be conducted throughout cycle 1 (28 days). Objective Response Rate (Complete Response (CR) and Partial Response (PR)) as determined by the patient's best tumor response, DoR, and PFS will be assessed using RECIST v1.1 by Investigator assessment. Tumor response assessments will continue until disease progression, initiation of new anticancer therapy, or study withdrawal. Overall survival will also be documented.

Radiological assessments of tumor lesions will be made at screening, at least once approximately every 8 weeks (+5 days) for the remainder of the first 12 months of study intervention (through and including Cycle 13), and once approximately every 12 weeks (+5 days) for year 2 and beyond of study intervention. Additional tumor assessments may be conducted.

Lesions to be included in the tumor assessments should follow RECIST v1.1. Computed tomography (CT) scan with a contrast agent is the preferred imaging method and the same technique should be used at screening and post-treatment assessments. CT scan coverage at screening should encompass scans of the chest and abdomen (including the liver and adrenals), and pelvis. Any other areas of disease involvement should be scanned based on the patient's signs and symptoms.

Pharmacokinetics and Pharmacodynamics. To evaluate the pharmacokinetics of the combination, blood samples will be collected at various timepoints and analyzed for area under the concentration-time curve (AUC), maximum plasma concentration, time to maximum observed concentration, terminal elimination rate constant, terminal half-life, apparent clearance, and apparent volume of distribution, for each agent. Plasma concentrations will be analyzed using a noncompartmental pharmacokinetic analysis (NCA).

Pharmacodynamic biomarkers and ctDNA will be evaluated. Biomarker analyses may include, but are not limited to: change from baseline in amount of farnesylated target proteins; correlation of pharmacokinetics with changes from baseline in farnesyltransferase enzyme activity and levels of farnesylated target proteins; change from baseline in the levels of EGFR mutation in ctDNA; time to ctDNA positivity; time to known genetic resistance in ctDNA; and presence of other genetic alterations in ctDNA. These evaluations will be performed using a combination of biochemical, genomic, transcriptomic, and proteomic technology, which may include profiling of mutation, amplification and/or other somatic gene alteration in DNA, RNA, or protein levels in tumor tissue.

Biomarkers in tumor tissue and blood will be studied for potential correlation between efficacy and/or treatment resistance and NSCLC biology (e.g., farnesylation status of target proteins, clearance rates).

### EXEMPLARY EMBODIMENTS

One or more than one (including for instance all) of the following exemplary Embodiments may comprise each of the other embodiments or parts thereof.
A1. A method of mitigating EGFR-TKI resistance in a subject, such as osimertinib resistance in a subject, comprising administering to the subject (a) a compound of Formula (I):

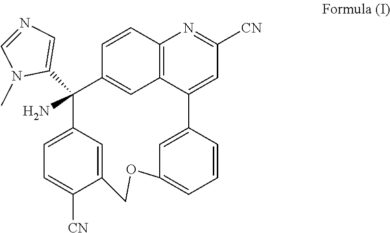 [SMILES: Cn1cncc1[C@@]1(N)c2ccc(C#N)c(c2)COc2cccc(c2)-c2cc(C#N)nc3ccc1cc23]

or a pharmaceutically acceptable form thereof, and (b) an EGFR-TKI, such as osimertinib, or a pharmaceutically acceptable salt thereof.
A2. A method of preventing or delaying emergence of EGFR-TKI resistance, such as osimertinib resistance in a subject, comprising administering to the subject (a) a compound of Formula (I):

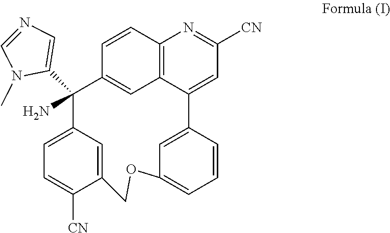 [SMILES: Cn1cncc1[C@@]1(N)c2ccc(C#N)c(c2)COc2cccc(c2)-c2cc(C#N)nc3ccc1cc23]

or a pharmaceutically acceptable form thereof, and (b) an EGFR-TKI, such as osimertinib, or a pharmaceutically acceptable salt thereof; wherein the subject is an EGFR-TKI-naïve subject, such as an osimertinib-naïve subject.
A3. A method of treating non-small cell lung cancer (NSCLC) in a subject, comprising administering to the subject (a) a compound of Formula (I):

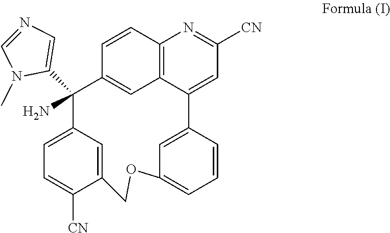 [SMILES: Cn1cncc1[C@@]1(N)c2ccc(C#N)c(c2)COc2cccc(c2)-c2cc(C#N)nc3ccc1cc23]

or a pharmaceutically acceptable form thereof, and (b) an EGFR-TKI, such as osimertinib, or a pharmaceutically acceptable salt thereof.
A4. The method of Embodiment A1 or Embodiment A2, wherein the method comprises administering to the subject a therapeutically effective amount of the compound of Formula (I) or pharmaceutically acceptable form thereof.
A5. The method of any one of Embodiments A1-A4, wherein the method comprises administering to the subject a therapeutically effective amount of the EGFR-TKI, such as the osimertinib or pharmaceutically acceptable salt thereof.
A6. A method of treating non-small cell lung cancer (NSCLC) in a subject, comprising administering to the subject (a) a pharmaceutical composition comprising a compound of Formula (I):

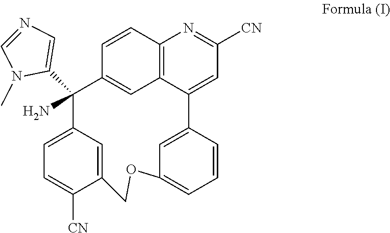 [SMILES: Cn1cncc1[C@@]1(N)c2ccc(C#N)c(c2)COc2cccc(c2)-c2cc(C#N)nc3ccc1cc23]

or a pharmaceutically acceptable form thereof, and a pharmaceutically acceptable carrier, excipient or diluent, and (b) a pharmaceutical composition comprising an EGFR-TKI, such as osimertinib, or a pharmaceutically acceptable salt thereof, and a pharmaceutically acceptable carrier, excipient or diluent.
A7. The method of Embodiment A6, wherein the pharmaceutical composition of (a) comprises a therapeutically effective amount of the compound of Formula (I) or pharmaceutically acceptable form thereof.
A8. The method of Embodiment A6 or Embodiment A7, wherein the pharmaceutical composition of (b) comprises a therapeutically effective amount of the osimertinib or pharmaceutically acceptable salt thereof.
A9. The method of any one of Embodiments A1-A8, wherein the NSCLC is an EGFR-mediated NSCLC, optionally wherein the EGFR-mediated NSCLC is an EGFR-mutant mediated NSCLC or the NSCLC has an EGFR mutation.
A10. The method of any one of Embodiments A1-A9, wherein NSCLC comprises an EGFR mutation selected from the group consisting of an exon 19 deletion, an exon 21 L858R mutation, a T790M mutation, an exon 20 insertion, and a C797S mutation.
A11. The method of any one of Embodiments A1-A10, wherein the NSCLC comprises two or more EGFR mutations.
A12. The method of any one of Embodiments A1-A11, wherein the NSCLC comprises an exon 19 deletion or an exon 21 L858R mutation, or both.
A13. The method of any one of Embodiments A1-A12, wherein the NSCLC comprises a T790M mutation.
A14. The method of any one of Embodiments A1-A13, wherein the NSCLC comprises an exon 20 insertion.
A15. The method of any one of Embodiments A1-A14, wherein the NSCLC comprises a C797S mutation.
A16. The method of any one of Embodiments A1-A15, wherein the NSCLC is early stage NSCLC.
A17. The method of any one of Embodiments A1-A15, wherein the NSCLC is an advanced or a metastatic NSCLC.
A18. The method of any one of Embodiments A1-A15, wherein the NSCLC is a relapsed or a refractory NSCLC.
A19. The method of any one of Embodiments A1-A18, wherein the subject has, suffers from, has symptoms associated with, or is diagnosed as having, NSCLC.
A20. The method of any one of Embodiments A1-A18, wherein the subject has or suffers from NSCLC.
A21. The method of any one of Embodiments A1-A20, wherein the subject is a NSCLC subject in remission.
A22. The method of any one of Embodiments A1-A21, wherein the subject is a tyrosine kinase inhibitor (TKI)-naïve subject.
A23. The method of any one of Embodiments A1-A22, wherein the EGFR-TKI is a second, third or fourth generation EGFR TKI.
A24. The method of any one of Embodiments A1-A23, wherein the EGFR-TKI is an irreversible EGFR-TKI.
A25. The method of any one of Embodiments A1-A24, wherein the EGFR-TKI is afatinib (Gilotrif), avitinib (AC0010), BIBW2992, BLU-945, dacomitinib (Vizimpro), EAI045, lazertinib (YH25448/GNS-1480), mavelertinib (PF-06747775; N-((3R,4R)-4-fluoro-1-(6-((3-methoxy-1-methyl-1H-pyrazol-4-yl)amino)-9-methyl-9H-purin-2-yl) pyrrolidine-3-yl) acrylamide), mobocertinib (TAK-788), nazartinib (EGF816), olmutinib (HM61713/BI 1482694), osimertinib (AZD9291), poziotinib (HM781-36B), rociletinib (CO-1686), or tarloxotinib (Tarlox), or a pharmaceutically acceptable form thereof.
A26. The method of any one of Embodiments A1-A25, wherein the EGFR-TKI is osimertinib or a pharmaceutically acceptable form thereof.
A27. The method of any one of Embodiments A1-A26, wherein the EGFR-TKI is osimertinib mesylate.
A28. The method of any one of Embodiments A1-A27, wherein the subject is an osimertinib-naïve subject.
A29. The method of any one of Embodiments A1-A27, wherein the subject has been treated previously with osimertinib.
A30. The method of any one of Embodiments A1-A29, wherein the osimertinib or pharmaceutically acceptable salt thereof is administered to the subject orally.
A31. The method of any one of Embodiments A1-A30, wherein the osimertinib or pharmaceutically acceptable salt thereof is administered to the subject at a dose of 10-160 mg.
A32. The method of any one of Embodiments A1-A31, wherein the osimertinib or pharmaceutically acceptable salt thereof is administered to the subject at a dose of about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, or about 160 mg.
A33. The method of any one of Embodiments A1-A32, wherein the osimertinib or pharmaceutically acceptable salt thereof is administered to the subject at a dose of 40 mg.
A34. The method of any one of Embodiments A1-A32, wherein the osimertinib or pharmaceutically acceptable salt thereof is administered to the subject at a dose of 80 mg.
A35. The method of any one of Embodiments A1-A34, wherein the osimertinib or pharmaceutically acceptable salt thereof is administered to the subject 1, 2, 3, or 4 times per day for one or more cycles.
A36. The method of any one of Embodiments A1-A35, wherein the osimertinib or pharmaceutically acceptable salt thereof is administered to the subject once or twice per day for one or more cycles.
A37. The method of any one of Embodiments A1-A36, wherein the compound of Formula (I) or pharmaceutically acceptable form thereof is administered to the subject orally.
A38. The method of any one of Embodiments A1-A37, wherein the compound of Formula (I) or pharmaceutically acceptable form thereof is administered at a dose of 0.01-50 mg/kg body weight per day.
A39. The method of any one of Embodiments A1-A38, wherein the compound of Formula (I) or pharmaceutically acceptable form thereof is administered at a dose of 1-2400 mg per day.
A40. The method of any one of Embodiments A1-A39, wherein the compound of Formula (I) or pharmaceutically acceptable form thereof is administered at a dose of 0.5-2.5 mg, 0.5-5 mg, 0.5-10 mg, 0.5-25 mg, 0.5-50 mg, 0.5-75 mg, 0.5-100 mg, 0.5-300 mg, 0.5-600 mg, 0.5-1200 mg, 1-5 mg, 1-10 mg, 1-25 mg, 1-50 mg, 1-75 mg, 1-100 mg, 1-300 mg, 1-600 mg, 1-1200 mg, 1-2400 mg, 20-100 mg, 40-75 mg, 50-75 mg, 50-100 mg, 50-150 mg, 75-100 mg, 100-200 mg, 125-200 mg, 150-300 mg, 200-250 mg, 200-400 mg, 300-600 mg, 250-500 mg, 400-600 mg, 500-750 mg, 600-900 mg, 700-100 mg, 650-1000 mg, 800-1200 mg, 900-1500 mg, 1000-1600 mg, 1000-2000 mg, 1200-1600 mg, 1500-2000 mg, 1500-2400 mg, 1800-2400 mg and 2000-2400 mg per day.
A41. The method of Embodiment A40, wherein the compound of Formula (I) or pharmaceutically acceptable form thereof is administered at a dose of about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, about 2050 mg, about 2100 mg, about 2150 mg, about 2200 mg, about 2250 mg, about 2300 mg, about 2350 mg, and about 2400 mg per day.
A42. The method of any one of Embodiments A1-A41, wherein the compound of Formula (I) or pharmaceutically acceptable form thereof is administered to the subject 1, 2, 3, or 4 times per day for one or more cycles.
A43. The method of any one of Embodiments A1-A42, wherein the compound of Formula (I) or pharmaceutically acceptable form thereof is administered to the subject once or twice per day for one or more cycles.
A44. The method of any one of Embodiments A35-A43, wherein a cycle is 1 day, 7 days, or 28 days.
A45. The method of Embodiment A44, wherein a cycle is 28 days.

Embodiments A46-A54 intentionally left blank
A55. The method of any one of Embodiments A1-A54, wherein the method further comprises a loading dosing cycle prior to one or more treating cycles.
A56. The method of Embodiment A55, wherein the loading dosing cycle comprises administering (a) a loading dose of a compound of Formula (I), or a pharmaceutically acceptable form thereof, and (b) a dose of osimertinib, or a pharmaceutically acceptable form thereof.
A57. The method of Embodiment A55 or Embodiment A56, wherein the loading dose of the compound of Formula (I), or pharmaceutically acceptable form thereof, is 1.1-10 times the dose administered during the one or more treating cycles.
A58. The method of any one of Embodiments A55-A57, wherein the loading dose of the compound of Formula (I), or pharmaceutically acceptable form thereof, is 1.1, 1.2, 1.3, 1.4, 1.5, 1.6, 1.7, 1.8, 1.9, 2, 3, 4, 5, 6, 7, 8, 9, or 10 times the dose administered during the one or more treating cycles.
A59. The method of any one of Embodiments A55-A58, wherein the loading dose of the compound of Formula (I), or pharmaceutically acceptable form thereof, is administered 1, 2, 3, or 4 times per day during the loading dosing cycle.
A60. The method of any one of Embodiments A55-A58, wherein the loading dose of the compound of Formula (I), or pharmaceutically acceptable form thereof, is administered once per day during the loading dosing cycle.
A61. The method of any one of Embodiments A55-A58, wherein the loading dose of the compound of Formula (I), or pharmaceutically acceptable form thereof, is administered twice per day during the loading dosing cycle.
A62. The method of any one of Embodiments A55-A61, wherein the loading dosing cycle is for 1 day, 2 days, 3 days, 4 days, 5 days, 6 days, 7 days, 14 days, 21 days, or 28 days.
A63. The method of any one of Embodiments A1-A62, wherein the compound of Formula (I) or pharmaceutically acceptable form thereof and the osimertinib or pharmaceutically acceptable salt thereof are administered concurrently or sequentially.
A64. The method of any one of Embodiments A1-A62, wherein the compound of Formula (I) or pharmaceutically acceptable form thereof is administered before the administration of the osimertinib or pharmaceutically acceptable salt thereof.
A65. The method of any one of Embodiments A1-A62, wherein the compound of Formula (I) or pharmaceutically acceptable form thereof is administered after the administration of the osimertinib or pharmaceutically acceptable salt thereof.
A66. The method of any one of Embodiments A1-A65, wherein the compound of Formula (I) or pharmaceutically acceptable form thereof is administered once or twice per day on days 1-7 of a 28-day treatment cycle and the osimertinib or pharmaceutically acceptable salt thereof is administered once per day of a 28-day treatment cycle.
A67. The method of any one of Embodiments A1-A65, wherein the compound of Formula (I) or pharmaceutically acceptable form thereof is administered once or twice per day on days 1-7 and 15-21 of a 28-day treatment cycle and the osimertinib or pharmaceutically acceptable salt thereof is administered once per day of a 28-day treatment cycle.
A68. The method of any one of Embodiments A1-A65, wherein the compound of Formula (I) or pharmaceutically acceptable form thereof is administered once or twice per day on days 1-21 of a 28-day treatment cycle and the osimertinib or pharmaceutically acceptable salt thereof is administered once per day of a 28-day treatment cycle.
A69. The method of any one of Embodiments A1-A65, wherein the compound of Formula (I) or pharmaceutically acceptable form thereof and the osimertinib or pharmaceutically acceptable salt thereof are administered once per day of a 28-day treatment cycle.
A70. The method of any one of Embodiments A1-A69, wherein the compound of Formula (I) or pharmaceutically acceptable form thereof and the osimertinib or pharmaceutically acceptable salt thereof act synergistically.
A71. The method of any one of Embodiments A1-A70, wherein the method delays the time to emergence of drug resistance or delays progression of drug resistance.
A72. The method of any one of Embodiments A1-A71, wherein the method delays the time to emergence of osimertinib resistance or delays progression of osimertinib resistance.
A73. The method of any one of Embodiments A1-A72, wherein the method delays, halts or prevents progression of NSCLC.
A74. The method of any one of Embodiments A1-A73, wherein the method delays, halts or prevents NSCLC tumor growth.
A75. The method of any one of Embodiments A1-A74, wherein the method reduces a primary NSCLC tumor.
A76. The method of any one of Embodiments A1-A75, wherein the method relieves one or more NSCLC tumor-related symptoms.
A77. The method of any one of Embodiments A1-A76, wherein the method delays appearance of primary or secondary NSCLC tumors.
A78. The method of any one of Embodiments A1-A77, wherein the method slows the development of primary or secondary NSCLC tumors.
A79. The method of any one of Embodiments A1-A78, wherein the method decreases the occurrence of primary or secondary NSCLC tumors.
A80. The method of any one of Embodiments A1-A79, wherein the method slows or decreases the severity of secondary effects associated with NSCLC.
A81. The method of any one of Embodiments A1-A80, wherein the method arrests NSCLC tumor growth and reduces NSCLC tumors.
A82. The method of any one of Embodiments A1-A81, wherein the method increases Time To Progression (TTP), Progression Free Survival (PFS), Event-free survival (EFS), Overall Survival (OS), overall response rate (ORR), or duration of response (DoR).
A83. The method of any one of Embodiments A1-A82, wherein the method increases TTP.
A84. The method of any one of Embodiments A1-A83, wherein the method increases PFS.
A85. The method of any one of Embodiments A1-A84, wherein the method increases EFS.
A86. The method of any one of Embodiments A1-A85, wherein the method increases OS.
A87. The method of any one of Embodiments A1-A86, wherein the method increases ORR.
A88. The method of any one of Embodiments A1-A87, wherein the method increases DoR.
A89. The method of any one of Embodiments A1-A88, wherein the method decreases time to response (TTR).
A90. The method of any one of Embodiments A1-A89, method mitigates osimertinib resistance.
A91. The method of any one of Embodiments A1-A90, wherein the method prevents or delays the emergence of osimertinib resistance.
A92. The method of any one of Embodiments A1-A91, wherein the method reduces or mitigates toxicities associated with osimertinib.
A93. The method of any one of Embodiments A1-A92, wherein the method improves the efficacy of osimertinib.
A94. The method of any one of Embodiments A1-A93, wherein the method delays, halts or prevents progression of NSCLC.
A95. The method of any one of Embodiments A1-A94, wherein the pharmaceutically acceptable salt of osimertinib is administered.
A96. The method of any one of Embodiments A1-A95, wherein the pharmaceutically acceptable salt of osimertinib is osimertinib mesylate.
A97. A method of treating an EGFR-mediated NSCLC in a subject comprising administering to the subject a compound of Formula (I) or a pharmaceutically acceptable form thereof.
A98. The method of Embodiment A97, wherein:
(a) the NSCLC is characterized as described in any one of Embodiments A9 to A18; or
(b) the compound of Formula (I) or pharmaceutically acceptable form thereof is administered as described in any one of Embodiments A38 to A45 or A55 to A62; or
(c) the method has the effect described in any one of Embodiments A73 to A89; or
(d) any combination of (a) to (c).
B1. A pharmaceutical composition, comprising (a) a compound of Formula (I):

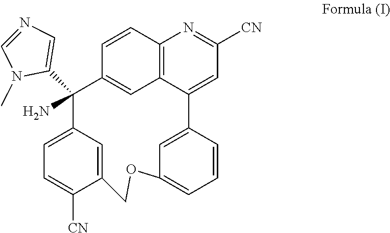 [SMILES: Cn1cncc1[C@@]1(N)c2ccc(C#N)c(c2)COc2cccc(c2)-c2cc(C#N)nc3ccc1cc23]

or a pharmaceutically acceptable form thereof, and (b) osimertinib, or a pharmaceutically acceptable salt thereof.
B2. A pharmaceutical kit, comprising (a) a compound of Formula (I):

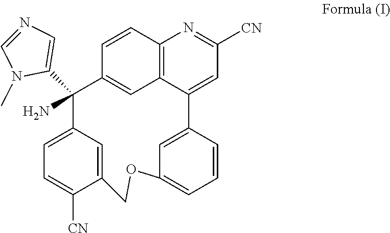 [SMILES: Cn1cncc1[C@@]1(N)c2ccc(C#N)c(c2)COc2cccc(c2)-c2cc(C#N)nc3ccc1cc23]

or a pharmaceutically acceptable form thereof, and (b) osimertinib, or a pharmaceutically acceptable salt thereof.
B3. A pharmaceutical packaging, comprising (a) a compound of Formula (I):

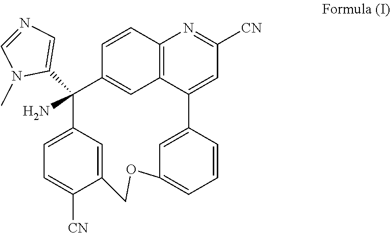 [SMILES: Cn1cncc1[C@@]1(N)c2ccc(C#N)c(c2)COc2cccc(c2)-c2cc(C#N)nc3ccc1cc23]

or a pharmaceutically acceptable form thereof, and (b) osimertinib, or a pharmaceutically acceptable salt thereof.
B4. The pharmaceutical composition, pharmaceutical kit, or pharmaceutical packaging of any one of Embodiments B1-B3, wherein the pharmaceutical composition, the pharmaceutical kit, or the pharmaceutical packaging comprises a therapeutically effective amount of the compound of Formula (I) or pharmaceutically acceptable form thereof.
B5. The pharmaceutical composition, pharmaceutical kit, or pharmaceutical packaging of any one of Embodiments B1-B4, wherein the pharmaceutical composition, the pharmaceutical kit, or the pharmaceutical packaging comprises 0.5-2400 mg of the compound of Formula (I) or pharmaceutically acceptable form thereof.
B6. The pharmaceutical composition, pharmaceutical kit, or pharmaceutical packaging of any one of Embodiments B1-B4, wherein the pharmaceutical composition, the pharmaceutical kit, or the pharmaceutical packaging comprises about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, about 2050 mg, about 2100 mg, about 2150 mg, about 2200 mg, about 2250 mg, about 2300 mg, about 2350 mg, and about 2400 mg of the compound of Formula (I) or pharmaceutically acceptable form thereof.
B7. The pharmaceutical composition, pharmaceutical kit, or pharmaceutical packaging of any one of Embodiments B1-B7, wherein the pharmaceutical composition, the pharmaceutical kit, or the pharmaceutical packaging comprises a therapeutically effective amount of the osimertinib or pharmaceutically acceptable salt thereof.
B8. The pharmaceutical composition, pharmaceutical kit, or pharmaceutical packaging of any one of Embodiments B1-B7, wherein the pharmaceutical composition, the pharmaceutical kit, or the pharmaceutical packaging comprises a dose of 10-160 mg of the osimertinib or pharmaceutically acceptable salt thereof.
B9. The pharmaceutical composition, pharmaceutical kit, or pharmaceutical packaging of any one of Embodiments B1-B7, wherein the pharmaceutical composition, the pharmaceutical kit, or the pharmaceutical packaging comprises a dose of about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, or about 160 mg of the osimertinib or pharmaceutically acceptable salt thereof.
B10. The pharmaceutical composition, pharmaceutical kit, or pharmaceutical packaging of any one of Embodiments B1-B7, wherein the pharmaceutical composition, the pharmaceutical kit, or the pharmaceutical packaging comprises a dose of 40 mg of the osimertinib or pharmaceutically acceptable salt thereof.
B11. The pharmaceutical composition, pharmaceutical kit, or pharmaceutical packaging of any one of Embodiments B1-B7, wherein the pharmaceutical composition, the pharmaceutical kit, or the pharmaceutical packaging comprises a dose of 80 mg of the osimertinib or pharmaceutically acceptable salt thereof.
B12. A pharmaceutical kit, comprising (a) a pharmaceutical composition comprising a compound of Formula (I):

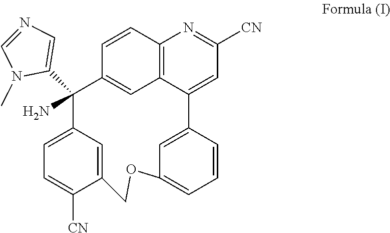 [SMILES: Cn1cncc1[C@@]1(N)c2ccc(C#N)c(c2)COc2cccc(c2)-c2cc(C#N)nc3ccc1cc23]

or a pharmaceutically acceptable form thereof, and a pharmaceutically acceptable carrier, excipient or diluent, and (b) a pharmaceutical composition comprising osimertinib, or a pharmaceutically acceptable salt thereof, and a pharmaceutically acceptable carrier, excipient or diluent.
B13. The pharmaceutical kit of Embodiment B12, wherein the pharmaceutical kit further comprises instructions for administering the contents of the kit to a subject having NSCLC.
B14. A pharmaceutical packaging, comprising (a) a pharmaceutical composition comprising a compound of Formula (I):

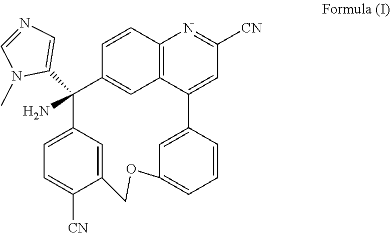 [SMILES: Cn1cncc1[C@@]1(N)c2ccc(C#N)c(c2)COc2cccc(c2)-c2cc(C#N)nc3ccc1cc23]

or a pharmaceutically acceptable form thereof, and a pharmaceutically acceptable carrier, excipient or diluent, and (b) a pharmaceutical composition comprising osimertinib, or a pharmaceutically acceptable salt thereof, and a pharmaceutically acceptable carrier, excipient or diluent.
B15. The pharmaceutical packaging of Embodiment B5, wherein the pharmaceutical packaging further comprises instructions for administering the contents of the packaging to a subject having NSCLC.
B16. The pharmaceutical composition or pharmaceutical kit of any one of Embodiments B1-B15, wherein the pharmaceutical composition of (a) comprises a therapeutically effective amount of the compound of Formula (I) or pharmaceutically acceptable form thereof.
B17. The pharmaceutical composition or pharmaceutical kit of any one of Embodiments B1-B15, wherein the pharmaceutical composition of (a) comprises 0.5-2400 mg of the compound of Formula (I) or pharmaceutically acceptable form thereof.
B18. The pharmaceutical composition or pharmaceutical kit of any one of Embodiments B1-B15, wherein the pharmaceutical composition of (a) comprises 0.5-2.5 mg, 0.5-5 mg, 0.5-10 mg, 0.5-25 mg, 0.5-50 mg, 0.5-75 mg, 0.5-100 mg, 0.5-300 mg, 0.5-600 mg, 0.5-1200 mg, 1-5 mg, 1-10 mg, 1-25 mg, 1-50 mg, 1-75 mg, 1-100 mg, 1-300 mg, 1-600 mg, 1-1200 mg, 1-2400 mg, 20-100 mg, 40-75 mg, 50-75 mg, 50-100 mg, 50-150 mg, 75-100 mg, 100-200 mg, 125-200 mg, 150-300 mg, 200-250 mg, 200-400 mg, 300-600 mg, 250-500 mg, 400-600 mg, 500-750 mg, 600-900 mg, 700-100 mg, 650-1000 mg, 800-1200 mg, 900-1500 mg, 1000-1600 mg, 1000-2000 mg, 1200-1600 mg, 1500-2000 mg, 1500-2400 mg, 1800-2400 mg and 2000-2400 mg of the compound of Formula (I) or pharmaceutically acceptable form thereof.
B19. The pharmaceutical composition or pharmaceutical kit of any one of Embodiments B1-B18, wherein the pharmaceutical composition of (b) comprises a therapeutically effective amount of the osimertinib or pharmaceutically acceptable salt thereof.
B20. The pharmaceutical composition or pharmaceutical kit of any one of Embodiments B1-B19, wherein the pharmaceutical composition of (b) comprises a dose of 10-160 mg of the osimertinib or pharmaceutically acceptable salt thereof.
B21. The pharmaceutical composition or pharmaceutical kit of any one of Embodiments B1-B19, wherein the pharmaceutical composition of (b) comprises a dose of about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, or about 160 mg of the osimertinib or pharmaceutically acceptable salt thereof.
B22. The pharmaceutical composition or pharmaceutical kit of any one of Embodiments B1-B19, wherein the pharmaceutical composition of (b) comprises a dose of 40 mg of the osimertinib or pharmaceutically acceptable salt thereof.
B23. The pharmaceutical composition or pharmaceutical kit of any one of Embodiments B1-B19, wherein the pharmaceutical composition of (b) comprises a dose of 80 mg of the osimertinib or pharmaceutically acceptable salt thereof.
B24. The pharmaceutical composition, pharmaceutical kit, or pharmaceutical packaging of any one of Embodiments B1-B23, wherein the pharmaceutically acceptable salt of osimertinib is osimertinib mesylate.
B25. The method of any one of Embodiments A1-A98, wherein the method administers the pharmaceutical composition, pharmaceutical kit, or pharmaceutical packaging of any one of Embodiments B1-B24.
C1. A method of mitigating osimertinib resistance in a subject, comprising administering to the subject (a) a compound of Formula (I):

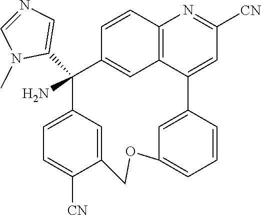 [SMILES: Cn1cncc1[C@@]1(N)c2ccc(C#N)c(c2)COc2cccc(c2)-c2cc(C#N)nc3ccc1cc23]

or a pharmaceutically acceptable form thereof, and (b) osimertinib, or a pharmaceutically acceptable salt thereof.
C2. A method of preventing or delaying emergence of osimertinib resistance in a subject, comprising administering to the subject (a) a compound of Formula (I):

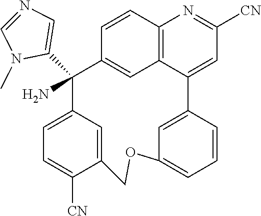 [SMILES: Cn1cncc1[C@@]1(N)c2ccc(C#N)c(c2)COc2cccc(c2)-c2cc(C#N)nc3ccc1cc23]

or a pharmaceutically acceptable form thereof, and (b) osimertinib, or a pharmaceutically acceptable salt thereof; wherein the subject is an osimertinib-naïve subject.
C3. A method of treating non-small cell lung cancer (NSCLC) in a subject, comprising administering to the subject (a) a compound of Formula (I):

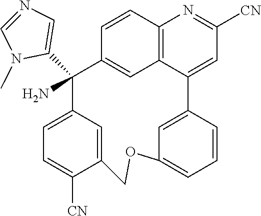 [SMILES: Cn1cncc1[C@@]1(N)c2ccc(C#N)c(c2)COc2cccc(c2)-c2cc(C#N)nc3ccc1cc23]

or a pharmaceutically acceptable form thereof, and (b) osimertinib, or a pharmaceutically acceptable salt thereof.
C4. The method of any one of Embodiments C1-C3, wherein the subject has NSCLC, suffers from NSCLC, has symptoms associated with NSCLC, is diagnosed as having NSCLC, or is a NSCLC subject in remission.
C5. The method of any one of Embodiments C1-C4, wherein the NSCLC has an EGFR mutation.
C6. The method of any one of Embodiments C1-C5, wherein the NSCLC comprises an EGFR mutation selected from the group consisting of an exon 19 deletion, an exon 21 L858R mutation, a T790M mutation, an exon 20 insertion, and a C797S mutation.
C7. The method of Embodiment C6, wherein the NSCLC comprises two or more EGFR mutations.
C8. The method of Embodiment C6 or Embodiment C7, wherein the NSCLC comprises an exon 19 deletion or an exon 21 L858R mutation, or both.
C9. The method of any one of Embodiments C6-C8, wherein the NSCLC comprises a T790M mutation.
C10. The method of any one of Embodiments C6-C9, wherein the NSCLC comprises an exon 20 insertion.
C11. The method of any one of Embodiments C6-C10, wherein the NSCLC comprises a C797S mutation.
C12. The method of any one of Embodiments C1-C11, wherein the NSCLC is early stage NSCLC.
C13. The method of any one of Embodiments C1-C11, wherein the NSCLC is an advanced or a metastatic NSCLC.
C14. The method of any one of Embodiments C1-C11, wherein the NSCLC is a relapsed or a refractory NSCLC.
C15. The method of any one of Embodiments C1-C14, wherein the subject is a tyrosine kinase inhibitor (TKI)-naïve subject.
C16. The method of any one of Embodiments C1-C14, wherein the subject is an osimertinib-naïve subject.
C17. The method of any one of Embodiments C1-C14, wherein the subject is currently being treated or has been previously treated with osimertinib.
C18. The method of any one of Embodiments C1-C17, wherein the osimertinib or pharmaceutically acceptable salt thereof is administered to the subject orally.
C19. The method of any one of Embodiments C1-C17, wherein the osimertinib or pharmaceutically acceptable salt thereof is administered to the subject at a dose of 10-160 mg.
C20. The method of Embodiment C19, wherein the osimertinib or pharmaceutically acceptable salt thereof is administered to the subject at a dose of 40 mg or 80 mg.
C21. The method of any one of Embodiments C1-C20, wherein the osimertinib or pharmaceutically acceptable salt thereof is administered to the subject once or twice per day for one or more cycles.
C22. The method of any one of Embodiments C1-C21, wherein the compound of Formula (I) or pharmaceutically acceptable form thereof is administered to the subject orally.
C23. The method of any one of Embodiments C1-C22, wherein the compound of Formula (I) or pharmaceutically acceptable form thereof is administered at a dose of 0.01-50 mg/kg body weight per day.
C24. The method of any one of Embodiments C1-C23, wherein the compound of Formula (I) or pharmaceutically acceptable form thereof is administered at a dose of 0.5-2400 mg per day.
C25. The method of Embodiment C24, wherein the compound of Formula (I) or pharmaceutically acceptable form thereof is administered at a dose of about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg about 100 mg, about 125 mg, about 150 mg, about 175 mg, about 200 mg, about 300 mg, about 350 mg, about 400 mg, about 450 mg, about 500 mg, about 550 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, and about 2400 mg per day.
C26. The method of any one of Embodiments C1-C25, wherein the compound of Formula (I) or pharmaceutically acceptable form thereof is administered 1, 2, 3, or 4 times per day for one or more cycles.
C27. The method of any one of Embodiments C21-C26, wherein a cycle is 1 day, 7 days, or 28 days.
C28. The method of Embodiment C27, wherein a cycle is 28 days.
C29. The method of any one of Embodiments C1-C28, wherein the compound of Formula (I) or pharmaceutically acceptable form thereof and the osimertinib or pharmaceutically acceptable salt thereof are administered concurrently or sequentially.
C30. The method of any one of Embodiments C1-C29, wherein the compound of Formula (I) or pharmaceutically acceptable form thereof is administered before the administration of the osimertinib or pharmaceutically acceptable salt thereof.
C31. The method of any one of Embodiments C1-C30, wherein the compound of Formula (I) or pharmaceutically acceptable form thereof is administered after the administration of the osimertinib or pharmaceutically acceptable salt thereof.
C32. The method of any one of Embodiments C1-C31, wherein the compound of Formula (I) or pharmaceutically acceptable form thereof is administered once or twice per day on days 1-7 of a 28-day treatment cycle and the osimertinib or pharmaceutically acceptable salt thereof is administered once per day of a 28-day treatment cycle.
C33. The method of any one of Embodiments C1-C31, wherein the compound of Formula (I) or pharmaceutically acceptable form thereof is administered once or twice per day on days 1-7 and 15-21 of a 28-day treatment cycle and the osimertinib or pharmaceutically acceptable salt thereof is administered once per day of a 28-day treatment cycle.
C34. The method of any one of Embodiments C1-C31, wherein the compound of Formula (I) or pharmaceutically acceptable form thereof is administered once or twice per day on days 1-21 of a 28-day treatment cycle and the osimertinib or pharmaceutically acceptable salt thereof is administered once per day of a 28-day treatment cycle.
C35. The method of any one of Embodiments C1-C31, wherein the compound of Formula (I) or pharmaceutically acceptable form thereof and the osimertinib or pharmaceutically acceptable salt thereof are administered once per day of a 28-day treatment cycle.
C36. The method of any one of Embodiments C1-C35, wherein the compound of Formula (I) or pharmaceutically acceptable form thereof and the osimertinib or pharmaceutically acceptable salt thereof act synergistically.
C37. The method of any one of Embodiments C1-C36, wherein the method mitigates osimertinib resistance.
C38. The method of any one of Embodiments C1-C37, wherein the method prevents or delays the emergence of osimertinib resistance.
C39. The method of any one of Embodiments C1-C38, wherein the method reduces or mitigates toxicities associated with osimertinib.
C40. The method of any one of Embodiments C1-C39, wherein the method improves the efficacy of osimertinib.
C41. The method of any one of Embodiments C1-C40, wherein the method delays, halts or prevents progression of NSCLC.
C42. The method of any one of Embodiments C1-C41, wherein the method increases Time To Progression (TTP), Progression Free Survival (PFS), Event-free survival (EFS), Overall Survival (OS), overall response rate (ORR), or duration of response (DoR), or combinations thereof.
C43. The method of any one of Embodiments C1-C42, wherein the method decreases time to response (TTR).
C44. The method of any one of Embodiments C1-C43, wherein the pharmaceutically acceptable salt of osimertinib is administered.
C45. The method of Embodiment C44, wherein the pharmaceutically acceptable salt of osimertinib is osimertinib mesylate.

The embodiments described above are intended to be merely exemplary, and those skilled in the art will recognize, or are able to ascertain using no more than routine experimentation, numerous equivalents of specific compounds, materials, and procedures. All such equivalents are considered to be within the scope of the invention and are encompassed by the appended claims.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference in its entirety. In case of conflict, the present application, including any definitions herein, will control.

What is claimed is:

1. A method of mitigating EGFR-TKI resistance in a subject, comprising administering to the subject (a) a compound of Formula (I): 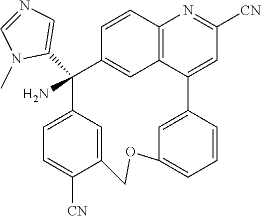 [SMILES: Cn1cncc1[C@@]1(N)c2ccc(C#N)c(c2)COc2cccc(c2)-c2cc(C#N)nc3ccc1cc23]
or a pharmaceutically acceptable form thereof, and (b) an EGFR-TKI.

2. A method of preventing or delaying emergence of EGFR-TKI resistance in a subject, comprising administering to the subject (a) a compound of Formula (I): 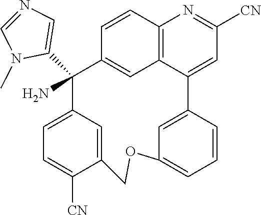 [SMILES: Cn1cncc1[C@@]1(N)c2ccc(C#N)c(c2)COc2cccc(c2)-c2cc(C#N)nc3ccc1cc23]
or a pharmaceutically acceptable form thereof, and (b) an EGFR-TKI; wherein the subject is an EGFR-TKI-naïve subject.

3. A method of treating non-small cell lung cancer (NSCLC) in a subject, comprising administering to the subject (a) a compound of Formula (I): 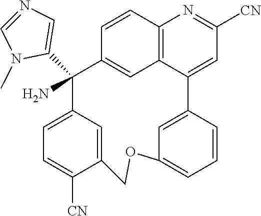 [SMILES: Cn1cncc1[C@@]1(N)c2ccc(C#N)c(c2)COc2cccc(c2)-c2cc(C#N)nc3ccc1cc23]
or a pharmaceutically acceptable form thereof, and (b) an EGFR-TKI.

4. The method of any one of claims 1-3, wherein the subject has NSCLC, suffers from NSCLC, has symptoms associated with NSCLC, is diagnosed as having NSCLC, or is a NSCLC subject in remission.

5. The method of any one of claims 1-4, wherein the NSCLC has an EGFR mutation.

6. The method of any one of claims 1-5, wherein the NSCLC comprises an EGFR mutation selected from the group consisting of an exon 19 deletion, an exon 21 L858R mutation, a T790M mutation, an exon 20 insertion, and a C797S mutation.

7. The method of claim 6, wherein the NSCLC comprises two or more EGFR mutations.

8. The method of claim 6 or claim 7, wherein the NSCLC comprises an exon 19 deletion or an exon 21 L858R mutation, or both.

9. The method of any one of claims 6-8, wherein the NSCLC comprises a T790M mutation.

10. The method of any one of claims 6-9, wherein the NSCLC comprises an exon 20 insertion.

11. The method of any one of claims 6-10, wherein the NSCLC comprises a C797S mutation.

12. The method of any one of claims 1-11, wherein the NSCLC is early stage NSCLC.

13. The method of any one of claims 1-11, wherein the NSCLC is an advanced or a metastatic NSCLC.

14. The method of any one of claims 1-11, wherein the NSCLC is a relapsed or a refractory NSCLC.

15. The method of any one of claims 1-14, wherein the subject is a tyrosine kinase inhibitor (TKI)-naïve subject.

16. The method of any one of claims 1-14, wherein the subject is an EGFR-TKI-naïve subject.

17. The method of any one of claims 1-14, wherein the subject is currently being treated or has been previously treated with an EGFR-TKI.

18. The method of any one of claims 1-17, wherein the EGFR-TKI is a second, third or fourth generation EGFR TKI.

19. The method of any one of claims 1-18, wherein the EGFR-TKI is an irreversible EGFR-TKI.

20. The method of any one of claims 1-19, wherein the EGFR-TKI is osimertinib or a pharmaceutically acceptable form thereof.

21. The method of claim 20, wherein the osimertinib or pharmaceutically acceptable salt thereof is administered to the subject orally.

22. The method of claim 20 or claim 21, wherein the osimertinib or pharmaceutically acceptable salt thereof is administered to the subject at a dose of 10-160 mg.

23. The method of claim 21, wherein the osimertinib or pharmaceutically acceptable salt thereof is administered to the subject at a dose of 40 mg or 80 mg.

24. The method of any one of claims 20-23, wherein the osimertinib or pharmaceutically acceptable salt thereof is administered to the subject once or twice per day for one or more cycles.

25. The method of any one of claims 1-24, wherein the compound of Formula (I) or pharmaceutically acceptable form thereof is administered to the subject orally.

26. The method of any one of claims 1-25, wherein the compound of Formula (I) or pharmaceutically acceptable form thereof is administered at a dose of 0.01-50 mg/kg body weight per day.

27. The method of any one of claims 1-26, wherein the compound of Formula (I) or pharmaceutically acceptable form thereof is administered at a dose of 0.5-2400 mg per day.

28. The method of claim 27, wherein the compound of Formula (I) or pharmaceutically acceptable form thereof is administered at a dose of about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg about 100 mg, about 125 mg, about 150 mg, about 175 mg, about 200 mg, about 300 mg, about 350 mg, about 400 mg, about 450 mg, about 500 mg, about 550 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, and about 2400 mg per day.

29. The method of any one of claims 1-28, wherein the compound of Formula (I) or pharmaceutically acceptable form thereof is administered 1, 2, 3, or 4 times per day for one or more cycles.

30. The method of any one of claims 24-29, wherein a cycle is 1 day, 7 days, or 28 days.

31. The method of claim 30, wherein a cycle is 28 days.

32. The method of any one of claims 20-31, wherein the compound of Formula (I) or pharmaceutically acceptable form thereof and the osimertinib or pharmaceutically acceptable salt thereof are administered concurrently or sequentially.

33. The method of any one of claims 20-32, wherein the compound of Formula (I) or pharmaceutically acceptable form thereof is administered before the administration of the osimertinib or pharmaceutically acceptable salt thereof.

34. The method of any one of claims 20-33, wherein the compound of Formula (I) or pharmaceutically acceptable form thereof is administered after the administration of the osimertinib or pharmaceutically acceptable salt thereof.

35. The method of any one of claims 20-34, wherein the compound of Formula (I) or pharmaceutically acceptable form thereof is administered once or twice per day on days 1-7 of a 28-day treatment cycle and the osimertinib or pharmaceutically acceptable salt thereof is administered once per day of a 28-day treatment cycle.

36. The method of any one of claims 20-34, wherein the compound of Formula (I) or pharmaceutically acceptable form thereof is administered once or twice per day on days 1-7 and 15-21 of a 28-day treatment cycle and the osimertinib or pharmaceutically acceptable salt thereof is administered once per day of a 28-day treatment cycle.

37. The method of any one of claims 20-34, wherein the compound of Formula (I) or pharmaceutically acceptable form thereof is administered once or twice per day on days 1-21 of a 28-day treatment cycle and the osimertinib or pharmaceutically acceptable salt thereof is administered once per day of a 28-day treatment cycle.

38. The method of any one of claims 20-34, wherein the compound of Formula (I) or pharmaceutically acceptable form thereof and the osimertinib or pharmaceutically acceptable salt thereof are administered once per day of a 28-day treatment cycle.

39. The method of any one of claims 20-38, wherein the compound of Formula (I) or pharmaceutically acceptable form thereof and the osimertinib or pharmaceutically acceptable salt thereof act synergistically.

40. The method of any one of claims 1-19, wherein the method mitigates EGFR-TKI resistance.

41. The method of any one of claims 1-19, wherein the method prevents or delays the emergence of EGFR-TKI resistance.

42. The method of any one of claims 1-19, wherein the method reduces or mitigates toxicities associated with the EGFR-TKI.

43. The method of any one of claims 1-19, wherein the method improves the efficacy of the EGFR-TKI.

44. The method of any one of claims 20-39, wherein the method mitigates osimertinib resistance.

45. The method of any one of claim 20-39 or 44, wherein the method prevents or delays the emergence of osimertinib resistance.

46. The method of any one of claim 20-39, 44, or 45, wherein the method reduces or mitigates toxicities associated with osimertinib.

47. The method of any one of claim 20-39 or 44-46, wherein the method improves the efficacy of osimertinib.

48. The method of any one of claims 1-47, wherein the method delays, halts or prevents progression of NSCLC.

49. The method of any one of claims 1-48, wherein the method increases Time To Progression (TTP), Progression Free Survival (PFS), Event-free survival (EFS), Overall Survival (OS), overall response rate (ORR), or duration of response (DoR), or combinations thereof.

50. The method of any one of claims 1-49, wherein the method decreases time to response (TTR).

51. The method of any one of claims 20-50, wherein the pharmaceutically acceptable salt of osimertinib is administered.

52. The method of claim 51, wherein the pharmaceutically acceptable salt of osimertinib is osimertinib mesylate.
